(19) United States
(12) Patent Application Publication
(10) Pub. No.: US 2009/0022670 A1
(43) Pub. Date: Jan. 22, 2009

(54) NOVEL COMPOUNDS

(76) Inventors: GIUSEPPE ALVARO, VERONA (IT); DAVID AMANTINI, VERONA (IT); SANDRO BELVEDERE, VERONA (IT)

Correspondence Address: GLAXOSMITHKLINE;Corporate Intellectual Property - UW2220, P.O. Box 1539, King of Prussia, PA, 19406-0939, US

(21) Appl. No.: 12/165,894

(22) Filed: Jul. 1, 2008

(30) Foreign Application Priority Data
Jul. 3, 2007 (GB) 0712887.9
Mar. 7, 2008 (GB) 0804317.6

(51) Int. Cl. C07D 401/14; A61K 9/12; A61K 31/437; A61P 25/00; A61P 25/20

(52) U.S. Cl. 424/45; 546/121; 514/300

(57) ABSTRACT

Disclosed are imidazo[1,2-a]pyridin-2-ylmethyl substituted piperidine derivatives of formula (I)

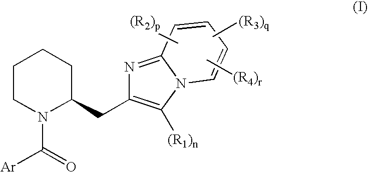

and their use as pharmaceuticals.

This application claims priority of Great Britain Application Nos. GB 0712887.9, filed 3 Jul. 2007, and GB 0804317.6, filed 7 Mar. 2006, which are incorporated herein in their entirety.

### BACKGROUND OF THE INVENTION

Many medically significant biological processes are mediated by proteins participating in signal transduction pathways that involve G-proteins and/or second messengers.

Polypeptides and polynucleotides encoding the human 7-transmembrane G-protein coupled neuropeptide receptor, orexin-1 (HFGAN72), have been identified and are disclosed in EP875565, EP875566 and WO 96/34877. Polypeptides and polynucleotides encoding a second human orexin receptor, orexin-2 (HFGANP), have been identified and are disclosed in EP893498.

Polypeptides and polynucleotides encoding polypeptides which are ligands for the orexin-1 receptor, e.g. orexin-A (Lig72A) are disclosed in EP849361.

The orexin ligand and receptor system has been well characterised since its discovery (see for example Sakurai, T. et al (1998) Cell, 92 pp 573 to 585; Smart et al (1999) British Journal of Pharmacology 128 pp 1 to 3; Willie et al (2001) Ann. Rev. Neurosciences 24 pp 429 to 458; Sakurai (2007) Nature Reviews Neuroscience 8 pp 171 to 181; Ohno and Sakurai (2008) Front. Neuroendocrinology 29 pp 70 to 87). From these studies it has become clear that orexins and orexin receptors play a number of important physiological roles in mammals and open up the possibility of the development of new therapeutic treatments for a variety of diseases and disorders as described hereinbelow.

Experiments have shown that central administration of the ligand orexin-A stimulated food intake in freely-feeding rats during a 4 hour time period. This increase was approximately four-fold over control rats receiving vehicle. These data suggest that orexin-A may be an endogenous regulator of appetite (Sakurai, T. et al (1998) Cell, 92 pp 573 to 585; Peyron et al (1998) J. Neurosciences 18 pp 9996 to 10015; Willie et al (2001) Ann. Rev. Neurosciences 24 pp 429 to 458). Therefore, antagonists of the orexin-A receptor(s) may be useful in the treatment of obesity and diabetes. In support of this it has been shown that orexin receptor antagonist SB334867 potently reduced hedonic eating in rats (White et al (2005) Peptides 26 pp 2231 to 2238) and also attenuated high-fat pellet self-administration in rats (Nair et al (2008) British Journal of Pharmacology, published online 28 Jan. 2008). The search for new therapies to treat obesity and other eating disorders is an important challenge. According to WHO definitions a mean of 35% of subjects in 39 studies were overweight and a further 22% clinically obese in westernised societies. It has been estimated that 5.7% of all healthcare costs in the USA are a consequence of obesity. About 85% of Type 2 diabetics are obese. Diet and exercise are of value in all diabetics. The incidence of diagnosed diabetes in westernised countries is typically 5% and there are estimated to be an equal number undiagnosed. The incidence of both diseases is rising, demonstrating the inadequacy of current treatments which may be either ineffective or have toxicity risks including cardiovascular effects. Treatment of diabetes with sulfonylureas or insulin can cause hypoglycemia, whilst metformin causes GI side-effects. No drug treatment for Type 2 diabetes has been shown to reduce the long-term complications of the disease. Insulin sensitizers will be useful for many diabetics, however they do not have an anti-obesity effect.

As well as having a role in food intake, the orexin system is also involved in sleep and wakefulness. Rat sleep/EEG studies have shown that central administration of orexin-A, an agonist of the orexin receptors, causes a dose-related increase in arousal, largely at the expense of a reduction in paradoxical sleep and slow wave sleep 2, when administered at the onset of the normal sleep period (Hagan et al (1999) Proc. Natl. Acad. Sci. 96 pp 10911 to 10916). The role of the orexin system in sleep and wakefulness is now well established (Sakurai (2007) Nature Reviews Neuroscience 8 pp 171 to 181; Ohno and Sakurai (2008) Front. Neuroendocrinology 29 pp 70 to 87; Chemelli et al (1999) Cell 98 pp 437 to 451; Lee et al (2005) J. Neuroscience 25 pp 6716 to 6720; Piper et al (2000) European J Neuroscience 12 pp 726-730 and Smart and Jerman (2002) Pharmacology and Therapeutics 94 pp 51 to 61). Antagonists of the orexin receptors may therefore be useful in the treatment of sleep disorders including insomnia. Studies with orexin receptor antagonists, for example SB334867, in rats (see for example Smith et al (2003) Neuroscience Letters 341 pp 256 to 258) and more recently dogs and humans (Brisbare-Roch et al (2007) Nature Medicine 13(2) pp 150 to 155) further support this.

In addition, recent studies have suggested a role for orexin antagonists in the treatment of motivational disorders, such as disorders related to reward seeking behaviours for example drug addiction and substance abuse (Borgland et al (2006) Neuron 49(4) pp 589-601; Boutrel et al (2005) Proc. Natl. Acad. Sci. 102(52) pp 19168 to 19173; Harris et al (2005) Nature 437 pp 556 to 559).

International Patent Applications WO99/09024, WO99/58533, WO00/47577 and WO00/47580 disclose phenyl urea derivatives and WO00/47576 discloses quinolinyl cinnamide derivatives as orexin receptor antagonists. WO05/118548 discloses substituted 1,2,3,4-tetrahydroisoquinoline derivatives as orexin antagonists.

WO01/96302, WO02/44172, WO02/89800, WO03/002559, WO03/002561, WO03/032991, WO03/037847, WO03/041711 and WO08/038,251 all disclose cyclic amine derivatives.

WO03/002561 discloses N-aroyl cyclic amine derivatives as orexin antagonists. Compounds disclosed in WO03/002561 include piperidine derivatives substituted at the 2-position with bicyclic heteroarylmethyl groups. We have now found that some piperidine derivatives substituted at the 2-position with an imidazo[1,2-a]pyridin-2-ylmethyl group have beneficial properties including, for example, increased oral bioavailability and significantly increased solubility in physiologically relevant media compared to the prior art compounds. Such properties make these imidazo[1,2-a]pyridin-2-ylmethyl substituted piperidine derivatives very attractive as potential pharmaceutical agents which may be useful in the prevention or treatment of obesity, including obesity observed in Type 2 (non-insulin-dependent) diabetes patients, sleep disorders, anxiety, depression, schizophrenia, drug dependency or compulsive behaviour. Additionally these compounds may be useful in the treatment of stroke, particularly ischemic or haemorrhagic stroke, and/or blocking the emetic response, i.e. useful in the treatment of nausea and vomiting.

### SUMMARY OF THE INVENTION

This invention relates to imidazo[1,2-a]pyridin-2-ylmethyl substituted piperidine derivatives and their use as pharmaceuticals. Accordingly the present invention provides a compound of formula (I)

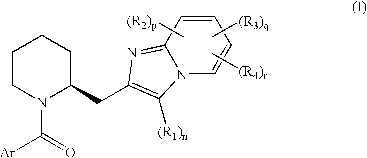

where Ar is selected from the group consisting of formulae:

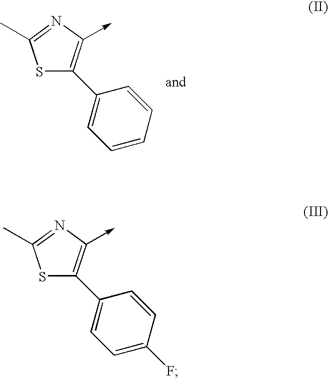

where

R₁ is (C₁₋₄)alkyl, halo, halo(C₁₋₄)alkyl, (C₁₋₄)alkoxy, halo(C₁₋₄)alkoxy, (C₁₋₄)alkyl-O—(C₁₋₄)alkyl, CN, NR⁵R⁶ wherein R⁵ is H or (C₁₋₄)alkyl and R⁶ is H or (C₁₋₄)alkyl;

R₂ is (C₁₋₄)alkyl, (C₁₋₄)alkenyl, HO(C₁₋₄)alkyl, halo, halo(C₁₋₄)alkyl, (C₁₋₄)alkoxy, halo(C₁₋₄)alkoxy, (C₁₋₄)alkyl-O—(C₁₋₄)alkyl, CN, NR⁷R⁸ wherein R⁷ is H or (C₁₋₄)-alkyl and R⁸ is H or (C₁₋₄)-alkyl;

R₃ is (C₁₋₄)alkyl, halo, halo(C₁₋₄)alkyl, (C₁₋₄)alkoxy, halo(C₁₋₄)alkoxy, (C₁₋₄)alkyl-O—(C₁₋₄)alkyl, CN, NR⁹R¹⁰ wherein R⁹ is H or (C₁₋₄)-alkyl and R¹⁰ is H or (C₁₋₄)-alkyl;

R⁴ is (C₁₋₄)alkyl, halo, halo(C₁₋₄)alkyl, (C₁₋₄)alkoxy, halo(C₁₋₄)alkoxy, (C₁₋₄)alkyl-O—(C₁₋₄)alkyl, CN, NR¹¹ R¹² wherein R¹¹ is H or (C₁₋₄)-alkyl and R¹² is H or (C₁₋₄)-alkyl;

n is 0 or 1;

p is 0 or 1;

q is 0 or 1;

r is 0 or 1;

or a pharmaceutically acceptable salt thereof.

### DETAILED DESCRIPTION OF THE INVENTION

In one embodiment:

R¹ is (C₁₋₄)alkyl, halo, halo(C₁₋₄)alkyl, (C₁₋₄)alkoxy, halo(C₁₋₄)alkoxy, (C₁₋₄)alkyl-O—(C₁₋₄)alkyl, CN, NR⁵R⁶ wherein R⁵ is H or (C₁₋₄)alkyl and R⁶ is H or (C₁₋₄)alkyl;

R₂ is (C₁₋₄)alkyl, halo, halo(C₁₋₄)alkyl, (C₁₋₄)alkoxy, halo(C₁₋₄)alkoxy, (C₁₋₄)alkyl-O—(C₁₋₄)alkyl, CN, NR⁷R⁸ wherein R⁷ is H or (C₁₋₄)-alkyl and R⁸ is H or (C₁₋₄)-alkyl;

R₃ is (C₁₋₄)alkyl, halo, halo(C₁₋₄)alkyl, (C₁₋₄)alkoxy, halo(C₁₋₄)alkoxy, (C₁₋₄)alkyl-O—(C₁₋₄)alkyl, CN, NR⁹R¹⁰ wherein R⁹ is H or (C₁₋₄)-alkyl and R¹⁰ is H or (C₁₋₄)-alkyl;

R⁴ is (C₁₋₄)alkyl, halo, halo(C₁₋₄)alkyl, (C₁₋₄)alkoxy, halo(C₁₋₄)alkoxy, (C₁₋₄)alkyl-O—(C₁₋₄)alkyl, CN, NR¹¹R¹² wherein R¹¹ is H or (C₁₋₄)-alkyl and R¹² is H or (C₁₋₄)-alkyl;

n is 0 or 1;

p is 0 or 1;

q is 0 or 1;

r is 0 or 1;

or a pharmaceutically acceptable salt thereof.

In one embodiment:

R₁ is (C₁₋₄)alkyl, halo, halo(C₁₋₄)alkyl or CN;

R₂ is (C₁₋₄)alkyl, (C₁₋₄)alkenyl, HO(C₁₋₄)alkyl, halo, halo(C₁₋₄)alkyl, (C₁₋₄)alkoxy, halo(C₁₋₄)alkoxy, (C₁₋₄)alkyl-O—(C₁₋₄)alkyl or CN;

R₃ is (C₁₋₄)alkyl, halo, halo(C₁₋₄)alkyl, (C₁₋₄)alkoxy, halo(C₁₋₄)alkoxy, (C₁₋₄)alkyl-O—(C₁₋₄)alkyl or CN;

R₄ is (C₁₋₄)alkyl, halo, halo(C₁₋₄)alkyl, (C₁₋₄)alkoxy, halo(C₁₋₄)alkoxy, (C₁₋₄)alkyl-O—(C₁₋₄)alkyl or CN;

n is 0 or 1;

p is 0 or 1;

q is 0 or 1;

r is 0 or 1;

or a pharmaceutically acceptable salt thereof.

In one embodiment Ar is a group of formula (II).

In another embodiment Ar is a group of formula (III).

In one embodiment n is 1 and R₁ is (C₁₋₄)alkyl or halo.

In another embodiment n is 1, R₁ is (C₁₋₄)alkyl or halo and Ar is a group of formula (II).

In a further embodiment n is 1, R₁ is methyl and Ar is a group of formula (II).

In a still further embodiment n is 1, R₁ is a halogen selected from fluoro, chloro or iodo and Ar is a group of formula (II).

In one embodiment n is 1, R₁ is methyl or a halogen selected from fluoro, chloro or iodo, Ar is a group of formula (II) and p, q and r are all 0.

In another embodiment n is 1, R₁ is methyl or a halogen selected from fluoro, chloro or iodo, Ar is a group of formula (II), p is 1 and q and r are both 0.

In a further embodiment n is 1, R₁ is methyl or a halogen selected from fluoro, chloro or iodo, Ar is a group of formula (II), p is 1, q and r are both 0 and R₂ is methyl, trifluoromethyl, fluoro or methyloxy.

In a still further embodiment n is 1, R₁ is chloro, Ar is a group of formula (II), p is 1, q and r are both 0 and R₂ is methyl or trifluoromethyl.

In one embodiment n is 0.

In another embodiment n is 0 and Ar is a group of formula (II).

In a further embodiment n is 0 and Ar is a group of formula (III).

In a still further embodiment n is 0, Ar is a group of formula (II) and r is 0.

In a yet still further embodiment n is 0, Ar is a group of formula (III) and r is 0.

In one embodiment n is 0, Ar is a group of formula (II), p and q are both 1 and r is 0.

In another embodiment n is 0, Ar is a group of formula (III), p and q are both 1 and r is 0.

In a further embodiment n is 0, Ar is a group of formula (II), p and q are both 1, r is 0 and R₂ and R₃ are both halo.

In a still further embodiment n is 0, Ar is a group of formula (III), p and q are both 1, r is 0 and R₂ and R₃ are both halo.

In a yet still further embodiment n is 0, Ar is a group of formula (II), p and q are both 1, r is 0 and R₂ and R₃ are both chloro.

In another embodiment n is 0, Ar is a group of formula (III), p and q are both 1, r is 0 and R₂ and R₃ are both chloro.

In a further embodiment n is 0, Ar is a group of formula (II), p and q are both 1, r is 0 and R₂ and R₃ are both fluoro.

In a still further embodiment n is 0, Ar is a group of formula (III), p and q are both 1, r is 0 and R₂ and R₃ are both fluoro.

In one embodiment n is 0, Ar is a group of formula (II), p and q are both 1, r is 0, R₂ is alkyl and R₃ is halo.

In another embodiment n is 0, Ar is a group of formula (II), p and q are both 1, r is 0, R₂ is alkyl in the 8 position on the imidazopyridine ring and R₃ is halo in the 6 position on the imidazopyridine ring.

In one embodiment n is 0, Ar is a group of formula (II), p and q are both 1, r is 0, R₂ is methyl and R₃ is fluoro.

In another embodiment n is 0, Ar is a group of formula (II), p and q are both 1, r is 0, R₂ is methyl in the 8 position on the imidazopyridine ring and R₃ is fluoro in the 6 position on the imidazopyridine ring.

In one embodiment n is 0, Ar is a group of formula (III), p and q are both 1, r is 0, R₂ is alkyl and R₃ is halo.

In another embodiment n is 0, Ar is a group of formula (III), p and q are both 1, r is 0, R₂ is alkyl in the 8 position on the imidazopyridine ring and R₃ is halo in the 6 position on the imidazopyridine ring.

In one embodiment n is 0, Ar is a group of formula (III), p and q are both 1, r is 0, R₂ is methyl and R₃ is fluoro.

In another embodiment n is 0, Ar is a group of formula (III), p and q are both 1, r is 0, R₂ is methyl in the 8 position on the imidazopyridine ring and R₃ is fluoro in the 6 position on the imidazopyridine ring.

In one embodiment n is 0, Ar is a group of formula (II), p is 1, q and r are both 0 and R₂ is (C₁₋₄)alkyl, halo, halo(C₁₋₄)alkyl, (C₁₋₄)alkoxy or CN.

In another embodiment n is 0, Ar is a group of formula (III), p is 1, q and r are both 0 and R₂ is (C₁₋₄)alkyl, halo, halo(C₁₋₄)alkyl, (C₁₋₄)alkoxy or CN.

In a further embodiment n is 0, Ar is a group of formula (II), p is 1, q and r are both 0 and R₂ is methyl, fluoro, trifluoromethyl, methyloxy or CN.

In a still further embodiment n is 0, Ar is a group of formula (III), p is 1, q and r are both 0 and R₂ is methyl, fluoro, trifluoromethyl, methyloxy or CN.

When the compound contains a (C₁₋₄)alkyl group, whether alone or forming part of a larger group, e.g. (C₁₋₄)alkoxy, the alkyl group may be straight chain, branched or cyclic, or combinations thereof. Examples of (C₁₋₄)alkyl are methyl or ethyl. An example of (C₁₋₄)alkoxy is methyloxy.

Examples of halo(C₁₋₄)alkyl include trifluoromethyl (i.e. —CF₃).

Examples of (C₁₋₄)alkoxy include methyloxy and ethyloxy.

Examples of halo(C₁₋₄)alkoxy include trifluoromethyloxy (i.e. —OCF₃).

Examples of (C₂₋₄)alkenyl include ethenyl.

Examples of HO(C₁₋₄)alkyl include hydroxymethyl.

Halogen or “halo” (when used, for example, in halo(C₁₋₄)alkyl) means fluoro, chloro, bromo or iodo.

It is to be understood that the present invention covers all combinations of particularized groups and substituents described herein above.

In one embodiment the invention provides the compound of formula (I) selected from the group consisting of:
2-[((2S)-1-{[5-(4-fluorophenyl)-2-methyl-1,3-thiazol-4-yl]carbonyl}-2-piperidinyl)methyl]-7-(trifluoromethyl)imidazo[1,2-a]pyridine;
2-({(2S)-1-[(2-methyl-5-phenyl-1,3-thiazol-4-yl)carbonyl]-2-piperidinyl}methyl)-7-(trifluoromethyl)imidazo[1,2-a]pyridine;
2-({(2S)-1-[(2-methyl-5-phenyl-1,3-thiazol-4-yl)carbonyl]-2-piperidinyl} methyl)-6-(trifluoromethyl)imidazo[1,2-a]pyridine;
2-({(2S)-1-[(2-methyl-5-phenyl-1,3-thiazol-4-yl)carbonyl]-2-piperidinyl}methyl)-8-(trifluoromethyl)imidazo[1,2-a]pyridine;
6,8-dichloro-2-({(2S)-1-[(2-methyl-5-phenyl-1,3-thiazol-4-yl)carbonyl]-2-piperidinyl} methyl)imidazo[1,2-a]pyridine;
8-methyl-2-({(2S)-1-[(2-methyl-5-phenyl-1,3-thiazol-4-yl)carbonyl]-2-piperidinyl}methyl)imidazo[1,2-a]pyridine;
6,8-difluoro-2-({(2S)-1-[(2-methyl-5-phenyl-1,3-thiazol-4-yl)carbonyl]-2-piperidinyl}methyl)imidazo[1,2-a]pyridine;
6-fluoro-2-({(2S)-1-[(2-methyl-5-phenyl-1,3-thiazol-4-yl)carbonyl]-2-piperidinyl} methyl)imidazo[1,2-a]pyridine;
2-({(2S)-1-[(2-methyl-5-phenyl-1,3-thiazol-4-yl)carbonyl]-2-piperidinyl} methyl)imidazo[1,2-a]pyridine-7-carbonitrile;
6-bromo-7,8-dimethyl-2-({(2S)-1-[(2-methyl-5-phenyl-1,3-thiazol-4-yl)carbonyl]-2-piperidinyl}methyl)imidazo[1,2-a]pyridine;
2-({(2S)-1-[(2-methyl-5-phenyl-1,3-thiazol-4-yl)carbonyl]-2-piperidinyl}methyl)-5-(trifluoromethyl)imidazo[1,2-a]pyridine;
6-bromo-5-methyl-2-({(2S)-1-[(2-methyl-5-phenyl-1,3-thiazol-4-yl)carbonyl]-2-piperidinyl}methyl)imidazo[1,2-a]pyridine;
8-fluoro-2-({(2S)-1-[(2-methyl-5-phenyl-1,3-thiazol-4-yl)carbonyl]-2-piperidinyl}methyl)imidazo[1,2-a]pyridine;
2-[((2S)-1-{[5-(4-fluorophenyl)-2-methyl-1,3-thiazol-4-yl]carbonyl}-2-piperidinyl)methyl]-8-methylimidazo[1,2-a]pyridine;
2-[((2S)-1-{[5-(4-fluorophenyl)-2-methyl-1,3-thiazol-4-yl]carbonyl}-2-piperidinyl)methyl]-8-(trifluoromethyl)imidazo[1,2-a]pyridine;
6,8-difluoro-2-[((2S)-1-{[5-(4-fluorophenyl)-2-methyl-1,3-thiazol-4-yl]carbonyl}-2-piperidinyl)methyl]imidazo[1,2-a]pyridine;
6,8-dichloro-2-[((2S)-1-{[5-(4-fluorophenyl)-2-methyl-1,3-thiazol-4-yl]carbonyl}-2-piperidinyl)methyl]imidazo[1,2-a]pyridine;
6-fluoro-2-[((2S)-1-{[5-(4-fluorophenyl)-2-methyl-1,3-thiazol-4-yl]carbonyl}-2-piperidinyl)methyl]imidazo[1,2-a]pyridine;
2-[((2S)-1-{[5-(4-fluorophenyl)-2-methyl-1,3-thiazol-4-yl]carbonyl}-2-piperidinyl)methyl]imidazo[1,2-a]pyridine-7-carbonitrile;
2-[((2S)-1-{[5-(4-fluorophenyl)-2-methyl-1,3-thiazol-4-yl]carbonyl}-2-piperidinyl)methyl]-7-(methyloxy)imidazo[1,2-a]pyridine;
2-[((2S)-1-{[5-(4-fluorophenyl)-2-methyl-1,3-thiazol-4-yl]carbonyl}-2-piperidinyl)methyl]imidazo[1,2-a]pyridine-8-carbonitrile;
5-fluoro-2-({(2S)-1-[(2-methyl-5-phenyl-1,3-thiazol-4-yl)carbonyl]-2-piperidinyl} methyl)imidazo[1,2-a]pyridine;
3-methyl-2-({(2S)-1-[(2-methyl-5-phenyl-1,3-thiazol-4-yl)carbonyl]-2-piperidinyl} methyl)imidazo[1,2-a]pyridine;
3-iodo-2-({(2S-1-[(2-methyl-5-phenyl-1,3-thiazol-4-yl)carbonyl]-2-piperidinyl} methyl)imidazo[1,2-a]pyridine;
3-chloro-2-({(2S)-1-[(2-methyl-5-phenyl-1,3-thiazol-4-yl)carbonyl]-2-piperidinyl}methyl)imidazo[1,2-a]pyridine;
3-chloro-2-({(2S)-1-[(2-methyl-5-phenyl-1,3-thiazol-4-yl)carbonyl]-2-piperidinyl}methyl)-7-(trifluoromethyl)imidazo[1,2-a]pyridine;
3-fluoro-8-methyl-2-({(2S)-1-[(2-methyl-5-phenyl-1,3-thiazol-4-yl)carbonyl]-2-piperidinyl}methyl)imidazo[1,2-a]pyridine;
3-chloro-6-fluoro-2-({(2S)-1-[(2-methyl-5-phenyl-1,3-thiazol-4-yl)carbonyl]-2-piperidinyl}methyl)imidazo[1,2-a]pyridine;
8-(methyloxy)-2-({(2S)-1-[(2-methyl-5-phenyl-1,3-thiazol-4-yl)carbonyl]-2-piperidinyl} methyl)imidazo[1,2-a]pyridine;
3-chloro-7-(methyloxy)-2-({(2S)-1-[(2-methyl-5-phenyl-1,3-thiazol-4-yl)carbonyl]-2-piperidinyl} methyl)imidazo[1,2-a]pyridine;
6-fluoro-8-methyl-2-({(2S)-1-[(2-methyl-5-phenyl-1,3-thiazol-4-yl)carbonyl]-2-piperidinyl}methyl)imidazo[1,2-a]pyridine;
8-ethenyl-6-fluoro-2-({(2S)-1-[(2-methyl-5-phenyl-1,3-thiazol-4-yl)carbonyl]-2-piperidinyl}methyl)imidazo[1,2-a]pyridine;
8-ethyl-6-fluoro-2-({(2S)-1-[(2-methyl-5-phenyl-1,3-thiazol-4-yl)carbonyl]-2-piperidinyl} methyl)imidazo[1,2-a]pyridine;
6-fluoro-8-(methyloxy)-2-({(2S)-1-[(2-methyl-5-phenyl-1,3-thiazol-4-yl)carbonyl]-2-piperidinyl}methyl)imidazo[1,2-a]pyridine;
[6-fluoro-2-({(2S)-1-[(2-methyl-5-phenyl-1,3-thiazol-4-yl)carbonyl]-2-piperidinyl}methyl)imidazo[1,2-a]pyridin-8-yl]methanol;
6-fluoro-8-[(methyloxy)methyl]-2-({(2S)-1-[(2-methyl-5-phenyl-1,3-thiazol-4-yl)carbonyl]-2-piperidinyl}methyl)imidazo[1,2-a]pyridine;
8-chloro-2-({(2S)-1-[(2-methyl-5-phenyl-1,3-thiazol-4-yl)carbonyl]-2-piperidinyl} methyl)imidazo[1,2-a]pyridine;
2-({(2S)-1-[(2-methyl-5-phenyl-1,3-thiazol-4-yl)carbonyl]-2-piperidinyl} methyl)-8-[(2,2,2-trifluoroethyl)oxy]imidazo[1,2-a]pyridine;
8-fluoro-2-[((2S)-1-{[5-(4-fluorophenyl)-2-methyl-1,3-thiazol-4-yl]carbonyl}-2-piperidinyl)methyl]imidazo[1,2-a]pyridine;
8-fluoro-3-methyl-2-({(2S)-1-[(2-methyl-5-phenyl-1,3-thiazol-4-yl)carbonyl]-2-piperidinyl}methyl)imidazo[1,2-a]pyridine;
8-fluoro-2-[((2S)-1-{[5-(4-fluorophenyl)-2-methyl-1,3-thiazol-4-yl]carbonyl}-2-piperidinyl)methyl]-3-methylimidazo[1,2-a]pyridine; and
3-chloro-8-methyl-2-({(2S)-1-[(2-methyl-5-phenyl-1,3-thiazol-4-yl)carbonyl]-2-piperidinyl} methyl)imidazo[1,2-a]pyridine;

or a pharmaceutically acceptable salt thereof.

In another embodiment the compound of formula (I) is 6-fluoro-8-methyl-2-({(2S)-1-[(2-methyl-5-phenyl-1,3-thiazol-4-yl)carbonyl]-2-piperidinyl}methyl)imidazo[1,2-a]pyridine or a pharmaceutically acceptable salt thereof.

In a further embodiment the compound of formula (I) is 6-fluoro-8-methyl-2-({(2S)-1-[(2-methyl-5-phenyl-1,3-thiazol-4-yl)carbonyl]-2-piperidinyl}methyl)imidazo[1,2-a]pyridine (HCl salt).

It will be appreciated that for use in medicine the salts of the compounds of formula (I) should be pharmaceutically acceptable. Suitable pharmaceutically acceptable salts will be apparent to those skilled in the art. Pharmaceutically acceptable salts include those described by Berge, Bighley and Monkhouse J. Pharm. Sci (1977) 66, pp 1-19. Such pharmaceutically acceptable salts include acid addition salts formed with inorganic acids e.g. hydrochloric, hydrobromic, sulphuric, nitric or phosphoric acid and organic acids e.g. succinic, maleic, acetic, fumaric, citric, tartaric, benzoic, p-toluenesulfonic, methanesulfonic or naphthalenesulfonic acid. Other salts e.g. oxalates or formates, may be used, for example in the isolation of compounds of formula (I) and are included within the scope of this invention.

Certain of the compounds of formula (I) may form acid addition salts with one or more equivalents of the acid. The present invention includes within its scope all possible stoichiometric and non-stoichiometric forms.

The compounds of formula (I) may be prepared in crystalline or non-crystalline form and, if crystalline, may optionally be solvated, eg. as the hydrate. This invention includes within its scope stoichiometric solvates (eg. hydrates) as well as compounds containing variable amounts of solvent (eg. water).

It will be understood that the invention includes pharmaceutically acceptable derivatives of compounds of formula (I) and that these are included within the scope of the invention.

As used herein “pharmaceutically acceptable derivative” includes any pharmaceutically acceptable ester or salt of such ester of a compound of formula (I) which, upon administration to the recipient is capable of providing (directly or indirectly) a compound of formula (I) or an active metabolite or residue thereof.

The compounds of formula (I) are S enantiomers. Where additional chiral centres are present in compounds of formula (I), the present invention includes within its scope all possible enantiomers and diastereoisomers, including mixtures thereof. The different isomeric forms may be separated or resolved one from the other by conventional methods, or any given isomer may be obtained by conventional synthetic methods or by stereospecific or asymmetric syntheses. The invention also extends to any tautomeric forms or mixtures thereof.

The subject invention also includes isotopically-labeled compounds which are identical to those recited in formula (I) but for the fact that one or more atoms are replaced by an atom having an atomic mass or mass number different from the atomic mass or mass number most commonly found in nature. Examples of isotopes that can be incorporated into compounds of the invention include isotopes of hydrogen, carbon, nitrogen, oxygen, fluorine, iodine and chlorine such as ³H, ¹¹C, ¹⁴C, ¹⁸F, ¹²³I or ¹²⁵I.

Compounds of the present invention and pharmaceutically acceptable salts of said compounds that contain the aforementioned isotopes and/or other isotopes of other atoms are within the scope of the present invention. Isotopically labeled compounds of the present invention, for example those into which radioactive isotopes such as ³H or ¹⁴C have been incorporated, are useful in drug and/or substrate tissue distribution assays. Tritiated, ie. ³H, and carbon-14, ie. ¹⁴C, isotopes are particularly preferred for their ease of preparation and detectability. ¹¹C and ¹⁸F isotopes are particularly useful in PET (positron emission tomography).

Since the compounds of formula (I) are intended for use in pharmaceutical compositions it will readily be understood that they are each preferably provided in substantially pure form, for example at least 60% pure, more suitably at least 75% pure and preferably at least 85%, especially at least 98% pure (% are on a weight for weight basis). Impure preparations of the compounds may be used for preparing the more pure forms used in the pharmaceutical compositions.

According to a further aspect of the present invention there is provided a process for the preparation of compounds of formula (I) and derivatives thereof. The following schemes detail some synthetic routes to compounds of the invention. In the following schemes reactive groups can be protected with protecting groups and deprotected according to well established techniques.

### Schemes

According to a further feature of the invention there is provided a process for the preparation of compounds of formula (I) or salts thereof. The following is an example of a synthetic scheme that may be used to synthesise the compounds of the invention.

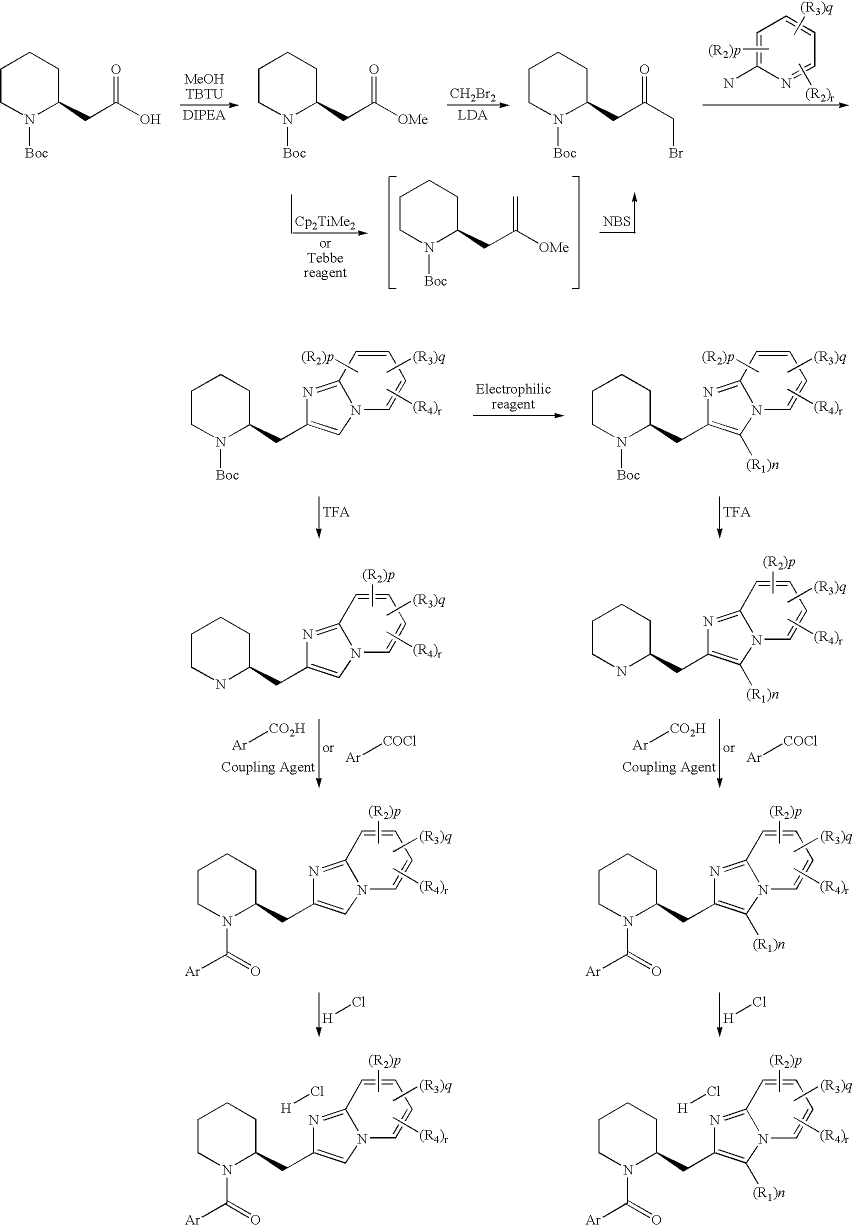

It will be understood by those skilled in the art that certain compounds of the invention can be converted into other compounds of the invention according to standard chemical methods.

The starting materials for use in the scheme are commercially available, known in the literature or can be prepared by known methods. The preparation of 5-phenyl-2-methyl-1,3-thiazole-4-carboxylic acids (the Ar groups) has been described in, for example, Mamedov et al (1991) Izvestiya Akademii Nauk SSSR, Seriya Khimicheskaya 12 pp 2832-2836. Mamedov et al (2004) Russian Journal of Organic Chemistry (Translation of Zhurnal Organicheskoi Khimii) 40(4) pp 534-542. ((2S)-1-{[(1,1-dimethylethyl)oxy]carbonyl}-2-piperidinyl)acetic acid is available from Neosystem Product List (BA19302).

Pharmaceutically acceptable salts may be prepared conventionally by reaction with the appropriate acid or acid derivative.

The present invention provides compounds of formula (I) or a pharmaceutically acceptable salt thereof for use in human or veterinary medicine.

The compounds of formula (I) or their pharmaceutically acceptable salts may be of use for the treatment or prophylaxis of a disease or disorder where an antagonist of a human orexin receptor is required such as sleep disorders selected from the group consisting of Dyssomnias such as Primary Insomnia (307.42), Primary Hypersomnia (307.44), Narcolepsy (347), Breathing-Related Sleep Disorders (780.59), Circadian Rhythm Sleep Disorder (307.45) and Dyssomnia Not Otherwise Specified (307.47); primary sleep disorders such as Parasomnias such as Nightmare Disorder (307.47), Sleep Terror Disorder (307.46), Sleepwalking Disorder (307.46) and Parasomnia Not Otherwise Specified (307.47); Sleep Disorders Related to Another Mental Disorder such as Insomnia Related to Another Mental Disorder (307.42) and Hypersomnia Related to Another Mental Disorder (307.44); Sleep Disorder Due to a General Medical Condition, in particular sleep disturbances associated with such diseases as neurological disorders, neuropathic pain, restless leg syndrome, heart and lung diseases; and Substance-Induced Sleep Disorder including the subtypes Insomnia Type, Hypersomnia Type, Parasomnia Type and Mixed Type; Sleep Apnea and Jet-Lag Syndrome.

In addition the compounds of formula (I) or their pharmaceutically acceptable salts may be of use for the treatment or prophylaxis of a disease or disorder where an antagonist of a human orexin receptor is required such as depression and mood disorders including Major Depressive Episode, Manic Episode, Mixed Episode and Hypomanic Episode; Depressive Disorders including Major Depressive Disorder, Dysthymic Disorder (300.4), Depressive Disorder Not Otherwise Specified (311); Bipolar Disorders including Bipolar I Disorder, Bipolar II Disorder (Recurrent Major Depressive Episodes with Hypomanic Episodes) (296.89), Cyclothymic Disorder (301.13) and Bipolar Disorder Not Otherwise Specified (296.80); Other Mood Disorders including Mood Disorder Due to a General Medical Condition (293.83) which includes the subtypes With Depressive Features, With Major Depressive-like Episode, With Manic Features and With Mixed Features), Substance-Induced Mood Disorder (including the subtypes With Depressive Features, With Manic Features and With Mixed Features) and Mood Disorder Not Otherwise Specified (296.90).

Further, the compounds of formula (I) or their pharmaceutically acceptable salts may be of use for the treatment or prophylaxis of a disease or disorder where an antagonist of a human orexin receptor is required such as anxiety disorders including Panic Attack; Panic Disorder including Panic Disorder without Agoraphobia (300.01) and Panic Disorder with Agoraphobia (300.21); Agoraphobia; Agoraphobia Without History of Panic Disorder (300.22), Specific Phobia (300.29, formerly Simple Phobia) including the subtypes Animal Type, Natural Environment Type, Blood-Injection-Injury Type, Situational Type and Other Type), Social Phobia (Social Anxiety Disorder, 300.23), Obsessive-Compulsive Disorder (300.3), Posttraumatic Stress Disorder (309.81), Acute Stress Disorder (308.3), Generalized Anxiety Disorder (300.02), Anxiety Disorder Due to a General Medical Condition (293.84), Substance-Induced Anxiety Disorder, Separation Anxiety Disorder (309.21), Adjustment Disorders with Anxiety (309.24) and Anxiety Disorder Not Otherwise Specified (300.00).

In addition the compounds of formula (I) or their pharmaceutically acceptable salts may be of use for the treatment or prophylaxis of a disease or disorder where an antagonist of a human orexin receptor is required such as substance-related disorders including Substance Use Disorders such as Substance Dependence, Substance Craving and Substance Abuse; Substance-Induced Disorders such as Substance Intoxication, Substance Withdrawal, Substance-Induced Delirium, Substance-Induced Persisting Dementia, Substance-Induced Persisting Amnestic Disorder, Substance-Induced Psychotic Disorder, Substance-Induced Mood Disorder, Substance-Induced Anxiety Disorder, Substance-Induced Sexual Dysfunction, Substance-Induced Sleep Disorder and Hallucinogen Persisting Perception Disorder (Flashbacks); Alcohol-Related Disorders such as Alcohol Dependence (303.90), Alcohol Abuse (305.00), Alcohol Intoxication (303.00), Alcohol Withdrawal (291.81), Alcohol Intoxication Delirium, Alcohol Withdrawal Delirium, Alcohol-Induced Persisting Dementia, Alcohol-Induced Persisting Amnestic Disorder, Alcohol-Induced Psychotic Disorder, Alcohol-Induced Mood Disorder, Alcohol-Induced Anxiety Disorder, Alcohol-Induced Sexual Dysfunction, Alcohol-Induced Sleep Disorder and Alcohol-Related Disorder Not Otherwise Specified (291.9); Amphetamine (or Amphetamine-Like)-Related Disorders such as Amphetamine Dependence (304.40), Amphetamine Abuse (305.70), Amphetamine Intoxication (292.89), Amphetamine Withdrawal (292.0), Amphetamine Intoxication Delirium, Amphetamine Induced Psychotic Disorder, Amphetamine-Induced Mood Disorder, Amphetamine-Induced Anxiety Disorder, Amphetamine-Induced Sexual Dysfunction, Amphetamine-Induced Sleep Disorder and Amphetamine-Related Disorder Not Otherwise Specified (292.9); Caffeine Related Disorders such as Caffeine Intoxication (305.90), Caffeine-Induced Anxiety Disorder, Caffeine-Induced Sleep Disorder and Caffeine-Related Disorder Not Otherwise Specified (292.9); Cannabis-Related Disorders such as Cannabis Dependence (304.30), Cannabis Abuse (305.20), Cannabis Intoxication (292.89), Cannabis Intoxication Delirium, Cannabis-Induced Psychotic Disorder, Cannabis-Induced Anxiety Disorder and Cannabis-Related Disorder Not Otherwise Specified (292.9); Cocaine-Related Disorders such as Cocaine Dependence (304.20), Cocaine Abuse (305.60), Cocaine Intoxication (292.89), Cocaine Withdrawal (292.0), Cocaine Intoxication Delirium, Cocaine-Induced Psychotic Disorder, Cocaine-Induced Mood Disorder, Cocaine-Induced Anxiety Disorder, Cocaine-Induced Sexual Dysfunction, Cocaine-Induced Sleep Disorder and Cocaine-Related Disorder Not Otherwise Specified (292.9); Hallucinogen-Related Disorders such as Hallucinogen Dependence (304.50), Hallucinogen Abuse (305.30), Hallucinogen Intoxication (292.89), Hallucinogen Persisting Perception Disorder (Flashbacks) (292.89), Hallucinogen Intoxication Delirium, Hallucinogen-Induced Psychotic Disorder, Hallucinogen-Induced Mood Disorder, Hallucinogen-Induced Anxiety Disorder and Hallucinogen-Related Disorder Not Otherwise Specified (292.9); Inhalant-Related Disorders such as Inhalant Dependence (304.60), Inhalant Abuse (305.90), Inhalant Intoxication (292.89), Inhalant Intoxication Delirium, Inhalant-Induced Persisting Dementia, Inhalant-Induced Psychotic Disorder, Inhalant-Induced Mood Disorder, Inhalant-Induced Anxiety Disorder and Inhalant-Related Disorder Not Otherwise Specified (292.9); Nicotine-Related Disorders such as Nicotine Dependence (305.1), Nicotine Withdrawal (292.0) and Nicotine-Related Disorder Not Otherwise Specified (292.9); Opioid-Related Disorders such as Opioid Dependence (304.00), Opioid Abuse (305.50), Opioid Intoxication (292.89), Opioid Withdrawal (292.0), Opioid Intoxication Delirium, Opioid-Induced Psychotic Disorder, Opioid-Induced Mood Disorder, Opioid-Induced Sexual Dysfunction, Opioid-Induced Sleep Disorder and Opioid-Related Disorder Not Otherwise Specified (292.9); Phencyclidine (or Phencyclidine-Like)-Related Disorders such as Phencyclidine Dependence (304.60), Phencyclidine Abuse (305.90), Phencyclidine Intoxication (292.89), Phencyclidine Intoxication Delirium, Phencyclidine-Induced Psychotic Disorder, Phencyclidine-Induced Mood Disorder, Phencyclidine-Induced Anxiety Disorder and Phencyclidine-Related Disorder Not Otherwise Specified (292.9); Sedative-, Hypnotic-, or Anxiolytic-Related Disorders such as Sedative, Hypnotic, or Anxiolytic Dependence (304.10), Sedative, Hypnotic, or Anxiolytic Abuse (305.40), Sedative, Hypnotic, or Anxiolytic Intoxication (292.89), Sedative, Hypnotic, or Anxiolytic Withdrawal (292.0), Sedative, Hypnotic, or Anxiolytic Intoxication Delirium, Sedative, Hypnotic, or Anxiolytic Withdrawal Delirium, Sedative-, Hypnotic-, or Anxiolytic-Persisting Dementia, Sedative-, Hypnotic-, or Anxiolytic-Persisting Amnestic Disorder, Sedative-, Hypnotic-, or Anxiolytic-Induced Psychotic Disorder, Sedative-, Hypnotic-, or Anxiolytic-Induced Mood Disorder, Sedative-, Hypnotic-, or Anxiolytic-Induced Anxiety Disorder Sedative-, Hypnotic-, or Anxiolytic-Induced Sexual Dysfunction, Sedative-, Hypnotic-, or Anxiolytic-Induced Sleep Disorder and Sedative-, Hypnotic-, or Anxiolytic-Related Disorder Not Otherwise Specified (292.9); Polysubstance-Related Disorder such as Polysubstance Dependence (304.80); and Other (or Unknown) Substance-Related Disorders such as Anabolic Steroids, Nitrate Inhalants and Nitrous Oxide.

In addition the compounds of formula (I) or their pharmaceutically acceptable salts may be of use for the treatment or prophylaxis of a disease or disorder where an antagonist of a human orexin receptor is required such as feeding disorders such as bulimia nervosa, binge eating, obesity, including obesity observed in Type 2 (non-insulin-dependent) diabetes patients. Further, the compounds of formula (I) or their pharmaceutically acceptable salts may be of use for the treatment or prophylaxis of a disease or disorder where an antagonist of a human orexin receptor is required such as stroke, particularly ischemic or haemorrhagic and/or in blocking an emetic response i.e. nausea and vomiting.

The numbers in brackets after the listed diseases refer to the classification code in DSM-IV: Diagnostic and Statistical Manual of Mental Disorders, 4th Edition, published by the American Psychiatric Association. The various subtypes of the disorders mentioned herein are contemplated as part of the present invention.

The invention also provides a method of treating or preventing a disease or disorder where an antagonist of a human orexin receptor is required, for example those diseases and disorders mentioned hereinabove, which comprises administering to a subject (e.g., a human) in need thereof an effective amount of a compound of formula (I) or a pharmaceutically acceptable salt thereof.

The invention also provides a compound of formula (I), or a pharmaceutically acceptable salt thereof, for use in the treatment or prophylaxis of a disease or disorder where an antagonist of a human orexin receptor is required, for example those diseases and disorders mentioned hereinabove.

The invention also provides the use of a compound of formula (I), or a pharmaceutically acceptable salt thereof, in the manufacture of a medicament for the treatment or prophylaxis of a disease or disorder where an antagonist of a human Orexin receptor is required, for example those diseases and disorders mentioned hereinabove.

For use in therapy the compounds of the invention are usually administered as a pharmaceutical composition. The invention also provides a pharmaceutical composition comprising a compound of formula (I), or a pharmaceutically acceptable salt thereof, and a pharmaceutically acceptable carrier.

The compounds of formula (I) or their pharmaceutically acceptable salts may be administered by any convenient method, e.g. by oral, parenteral, buccal, sublingual, nasal, rectal or transdermal administration, and the pharmaceutical compositions adapted accordingly.

The compounds of formula (I) or their pharmaceutically acceptable salts which are active when given orally can be formulated as liquids or solids, e.g. as syrups, suspensions, emulsions, tablets, capsules or lozenges.

A liquid formulation will generally consist of a suspension or solution of the active ingredient in a suitable liquid carrier(s) e.g. an aqueous solvent such as water, ethanol or glycerine, or a non-aqueous solvent, such as polyethylene glycol or an oil. The formulation may also contain a suspending agent, preservative, flavouring and/or colouring agent.

A composition in the form of a tablet can be prepared using any suitable pharmaceutical carrier(s) routinely used for preparing solid formulations, such as magnesium stearate, starch, lactose, sucrose and cellulose.

A composition in the form of a capsule can be prepared using routine encapsulation procedures, e.g. pellets containing the active ingredient can be prepared using standard carriers and then filled into a hard gelatin capsule; alternatively a dispersion or suspension can be prepared using any suitable pharmaceutical carrier(s), e.g. aqueous gums, celluloses, silicates or oils and the dispersion or suspension then filled into a soft gelatin capsule.

Typical parenteral compositions consist of a solution or suspension of the active ingredient in a sterile aqueous carrier or parenterally acceptable oil, e.g. polyethylene glycol, polyvinyl pyrrolidone, lecithin, arachis oil or sesame oil. Alternatively, the solution can be lyophilised and then reconstituted with a suitable solvent just prior to administration.

Compositions for nasal administration may conveniently be formulated as aerosols, drops, gels and powders. Aerosol formulations typically comprise a solution or fine suspension of the active ingredient in a pharmaceutically acceptable aqueous or non-aqueous solvent and are usually presented in single or multidose quantities in sterile form in a sealed container which can take the form of a cartridge or refill for use with an atomizing device. Alternatively the sealed container may be a disposable dispensing device such as a single dose nasal inhaler or an aerosol dispenser fitted with a metering valve. Where the dosage form comprises an aerosol dispenser, it will contain a propellant which can be a compressed gas e.g. air, or an organic propellant such as a fluorochlorohydrocarbon or hydrofluorocarbon. Aerosol dosage forms can also take the form of pump-atomizers.

Compositions suitable for buccal or sublingual administration include tablets, lozenges and pastilles where the active ingredient is formulated with a carrier such as sugar and acacia, tragacanth, or gelatin and glycerin.

Compositions for rectal administration are conveniently in the form of suppositories containing a conventional suppository base such as cocoa butter.

Compositions suitable for transdermal administration include ointments, gels and patches.

In one embodiment the composition is in unit dose form such as a tablet, capsule or ampoule.

The dose of the compound of formula (I), or a pharmaceutically acceptable salt thereof, used in the treatment or prophylaxis of the abovementioned disorders or diseases will vary in the usual way with the particular disorder or disease being treated, the weight of the subject and other similar factors. However, as a general rule, suitable unit doses may be 0.05 to 1000 mg, more suitably 0.05 to 500 mg. Unit doses may be administered more than once a day for example two or three times a day, so that the total daily dosage is in the range of about 0.01 to 100 mg/kg. Such therapy may extend for a number of weeks or months. In the case of pharmaceutically acceptable derivatives the above figures are calculated as the parent compound of formula (I).

Orexin-A (Sakurai, T. et al (1998) Cell, 92 pp 573-585)) can be employed in screening procedures for compounds which inhibit the ligand's activation of the orexin-1 or orexin-2 receptors.

In general, such screening procedures involve providing appropriate cells which express the orexin-1 or orexin-2 receptor on their surface. Such cells include cells from mammals, yeast, Drosophila or E. coli. In particular, a polynucleotide encoding the orexin-1 or orexin-2 receptor is used to transfect cells to express the receptor. The expressed receptor is then contacted with a test compound and an orexin-1 or orexin-2 receptor ligand, as appropriate, to observe inhibition of a functional response. One such screening procedure involves the use of melanophores which are transfected to express the orexin-1 or orexin-2 receptor, as described in WO 92/01810.

Another screening procedure involves introducing RNA encoding the orexin-1 or orexin-2 receptor into Xenopus oocytes to transiently express the receptor. The receptor oocytes are then contacted with a receptor ligand and a test compound, followed by detection of inhibition of a signal in the case of screening for compounds which are thought to inhibit activation of the receptor by the ligand.

Another method involves screening for compounds which inhibit activation of the receptor by determining inhibition of binding of a labelled orexin-1 or orexin-2 receptor ligand to cells which have the orexin-1 or orexin-2 receptor (as appropriate) on their surface. This method involves transfecting a eukaryotic cell with DNA encoding the orexin-1 or orexin-2 receptor such that the cell expresses the receptor on its surface and contacting the cell or cell membrane preparation with a compound in the presence of a labelled form of an orexin-1 or orexin-2 receptor ligand. The ligand may contain a radioactive label. The amount of labelled ligand bound to the receptors is measured, e.g. by measuring radioactivity.

Yet another screening technique involves the use of FLIPR equipment for high throughput screening of test compounds that inhibit mobilization of intracellular calcium ions, or other ions, by affecting the interaction of an orexin-1 or orexin-2 receptor ligand with the orexin-1 or orexin-2 receptor as appropriate.

Throughout the specification and claims which follow, unless the context requires otherwise, the word ‘comprise’, and variations such as ‘comprises’ and ‘comprising’ will be understood to imply the inclusion of a stated integer or step or group of integers but not to the exclusion of any other integer or step or group of integers or steps.

All publications, including but not limited to patents and patent applications, cited in this specification are herein incorporated by reference as if each individual publication were specifically and individually indicated to be incorporated by reference herein as though fully set forth.

The following Examples illustrate the preparation of certain compounds of formula (I) or salts thereof. The Descriptions 1 to 63 illustrate the preparation of intermediates used to make compounds of formula (I) or salts thereof.

In the procedures that follow, after each starting material, reference to a description is typically provided. This is provided merely for assistance to the skilled chemist. The starting material may not necessarily have been prepared from the Description referred to.

The yields were calculated assuming that products were 100% pure if not stated otherwise.

The compounds described in the Examples described hereinafter have all been prepared as a first step from stereochemically pure ((2S)-1-{[(1,1-dimethylethyl)oxy]carbonyl}-2-piperidinyl)acetic acid. The stereochemistry of the compounds of the Descriptions and Examples have been assigned on the assumption that the pure configuration is maintained.

Compounds are named using ACD/Name PRO6.02 chemical naming software (Advanced Chemistry Development Inc., Toronto, Ontario, M5H2L3, Canada).

Proton Magnetic Resonance (NMR) spectra were recorded either on Varian instruments at 400, 500 or 600 MHz, or on a Bruker instrument at 400 MHz. Chemical shifts are reported in ppm (δ) using the residual solvent line as internal standard. Splitting patterns are designed as s, singlet; d, doublet; t, triplet; q, quartet; m, multiplet; b, broad. The NMR spectra were recorded at a temperature ranging from 25 to 90° C. When more than one conformer was detected the chemical shifts for the most abundant one is usually reported.

Unless otherwise specified, HPLC analyses indicated by HPLC (walk-up): rt (retention time)=x min, were performed on a Agilent 1100 series instrument using a Luna 3u C18(2) 100A column (50×2.0 mm, 3 μm particle size) [Mobile phase and Gradient: 100% (water+0.05% TFA) to 95% (acetonitrile+0.05% TFA) in 8 min. Column T=40° C. Flow rate=1 mL/min. UV detection wavelength=220 nm]. Other HPLC analyses, indicated by HPLC (walk-up, 3 min method), were performed using an Agilent Zorbax SB-C18 column (50×3.0 mm, 1.8 μm particle size) [Mobile phase and Gradient: 100% (water+0.05% TFA) to 95% (acetonitrile+0.05% TFA) in 2.5 min, hold 0.5 min. Column T=60° C. Flow rate=1.5 mL/min. UV detection wavelength=220 nm].

Direct infusion Mass spectra (MS) were run on a Agilent MSD 1100 Mass Spectrometer, operating in ES (+) and ES (−) ionization mode [ES (+): Mass range: 100-1000 amu. Infusion solvent: water+0.1% HCO₂H/CH₃CN 50/50. ES (−): Mass range: 100-1000 amu. Infusion solvent: water+0.05% NH₄OH/CH₃CN 50/50] or on an Agilent LC/MSD 1100 Mass Spectrometer coupled with HPLC instrument Agilent 1100 Series, operating in positive or negative electrospray ionization mode and in both acidic and basic gradient conditions [Acidic gradient LC/MS−ES (+ or −): analyses performed on a Supelcosil ABZ+Plus column (33×4.6 mm, 3 μm). Mobile phase: A−water+0.1% HCO₂H/B−CH₃CN. Gradient (standard method): t=0 min 0% (B), from 0% (B) to 95% (B) in 5 min lasting for 1.5 min, from 95% (B) to 0% (B) in 0.1 min, stop time 8.5 min. Column T=room temperature. Flow rate=1 mL/min. Gradient (fast method): t=0 min 0% (B), from 0% (B) to 95% (B) in 3 min lasting for 1 min, from 95% (B) to 0% (B) in 0.1 min, stop time 4.5 min. Column T=room temperature. Flow rate=2 mL/min.

Basic gradient LC/MS−ES (+ or −): analyses performed on a XTerra MS C18 column (30×4.6 mm, 2.5 μm). Mobile phase: A −5 mM aq. NH₄HCO₃+ammonia (pH 10)/B−CH₃CN. Gradient: t=0 min 0% (B), from 0% (B) to 50% (B) in 0.4 min, from 50% (B) to 95% (B) in 3.6 min lasting for 1 min, from 95% (B) to 0% (B) in 0.1 min, stop time 5.8 min. Column T=room temperature. Flow rate=1.5 mL/min].

Mass range ES (+ or −): 100-1000 amu. UV detection range: 220-350 nm. The usage of this methodology is indicated by “LC-MS” in the analytic characterization of the described compounds.

Total ion current (TIC) and DAD UV chromatographic traces together with MS and UV spectra associated with the peaks were taken on a HPLC/MS Acquity™ system equipped with 2996 PDA detector and coupled to a Waters Micromass ZQ™ mass spectrometer operating in positive or negative electrospray ionisation mode [LC/MS−ES (+ or −): analyses performed using an Acquity™ HPLC BEH C18 column (50×2.1 mm, 1.7 μm particle size). Mobile phase: A−water+0.1% HCO₂H/B−CH₃CN+0.06% HCO₂H. Gradient: t=0 min 3% B, t=0.05 min 6% B, t=0.57 min 70% B, t=1.06 min 99% B lasting for 0.389 min, t=1.45 min 3% B, stop time 1.5 min. Column T=40° C. Flow rate=1.0 mL/min. Mass range: ES (+): 100-1000 amu. ES (−): 100-800 amu. UV detection range: 210-350 nm. The usage of this methodology is indicated by “HPLC” in the analytic characterization of the described compounds.

Unless otherwise specified, Preparative LC-MS purifications were run on a MDAP (Mass Detector Auto Purification) Waters instrument (MDAP FractionLynx). [LC/MS−ES (+): analyses performed using a Gemini C18 AXIA column (50×21 mm, 5 μm particle size). Mobile phase: A−NH₄HCO₃ sol. 10 mM, pH 10; B−CH₃CN. Flow rate: 17 mL/min. The gradient will be specified each time].

Preparative LC-MS purifications were also run on a MDAP (Mass Detector Auto Purification) Waters instrument. The usage of this methodology is indicated by “Fraction Lynx” in the analytic characterization of the described compounds.

LC3_{—}100 mg method. Column: Waters XTerra Prep MS C18 OBD (30×150 mm, 10 μm particle size). Mobile phase: A−H₂O+0.1% HCO₂H/B−CH₃CN+0.1% HCO₂H. Gradient: 30% to 55% (B) in 10 min, 55% to 99% (B) in 4 min, 99% to 100% (B) in 1 min. Flow rate=40 mL/min. UV detection range: 210-400 nm. Ionization: ES+/ES−. Mass range: 150-900 amu].

For reactions involving microwave irradiation, a Personal Chemistry Emrys™ Optimizer was used.

In a number of preparations, purification was performed using Biotage manual flash chromatography (Flash+), Biotage automatic flash chromatography (Horizon, SP1 and SP4), Companion CombiFlash (ISCO) automatic flash chromatography, Flash Master Personal or Vac Master systems.

Flash chromatography was carried out on silica gel 230-400 mesh (supplied by Merck AG Darmstadt, Germany), Varian Mega Be—Si pre-packed cartridges, pre-packed Biotage silica cartridges (e.g. Biotage SNAP cartridge), KP—NH prepacked flash cartridges or ISCO RediSep Silica cartridges.

SPE-SCX cartridges are ion exchange solid phase extraction columns supplied by Varian. The eluent used with SPE-SCX cartridges is methanol followed by 2N ammonia solution in methanol.

SPE-Si cartridges are silica solid phase extraction columns supplied by Varian.

The following table lists the used abbreviations:
AcCl Acetyl chloride
BINAP 2,2′-Bis(diphenylphosphino)-1,1′-binaphthyl
Boc t-Butoxycarbonyl
n-BuLi n-Butyl lithium
Cp Cyclopentadienyl
Cy Cyclohexanes
DBA Dibenzylidene acetone
DCM Dichloromethane
DIPA N,N-Diisopropylamine
DIPEA N,N-Diisopropyl-N-ethylamine
DME 1,2-Dimethoxyethane
DMF Dimethylformamide
EtOH Ethanol
Et₂O Diethylether
EtOAc Ethylacetate
IPA Isopropyl alcohol
LAH Lithium aluminum hydride
LDA Lithiumdiisopropylamide
MeOH Methanol
MsCl Mesylchloride
NBS N-Bromosuccinimide
NCS N-Chlorosuccinimide
Ps-TsCl Polystyrene sulfonyl chloride (cross-linked polystyrene resin that is the resin-bound equivalent of tosyl chloride)
rt retention time
T temperature
TBME tert-Butyl methyl ether
TBS tert-Butyl dimethylsilyl
TBTU O-(benzotriazol-1-yl)-N,N,N′N′-tetramethyluronium tetrafluoroborate
TEA Triethylamine
TFA Trifluoroacetic acid
THF Tetrahydrofuran

### DESCRIPTIONS

### Description 1

### 1,1-dimethylethyl (2S)-2-[2-(methyloxy)-2-oxoethyl]-1-piperidinecarboxylate (D1)

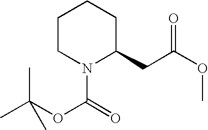

A mixture of ((2S)-1-{[(1,1-dimethylethyl)oxy]carbonyl}-2-piperidinyl)acetic acid (1.00 g, 4.11 mmol), DIPEA (2.148 ml, 12.33 mmol) and TBTU (1.979 g, 6.17 mmol) in DMF (25 ml) was stirred at room temperature for 20 min and a brown colour was formed. After this time MeOH (0.249 ml, 6.17 mmol) was added and the resulting solution stirred at room temperature for 30 min. The mixture was transferred into a separatory funnel containing brine (20 ml) and extracted with EtOAc (2×20 ml). The combined organic layers were washed with water/ice (5×20 ml). The organic layer was dried (Na₂SO₄), filtered and concentrated. The crude obtained was purified by flash chromatography on silica gel (Biotage SP 1, Cy/EtOAc from 100/0 to 85/15). Collected fractions gave the title compound D1 (1.01 g, 3.92 mmol, 95% yield) as a colorless oil.

¹H-NMR (400 MHz, CDCl₃) δ (ppm): 4.67-4.75 (m, 1H), 3.96-4.05 (m, 1H), 3.67 (s, 3H), 2.79 (t, 1H), 2.61 (dd, 1H), 2.53 (dd, 1H), 1.60-1.70 (m, 6H), 1.46 (s, 9H).

### Description 2

### 1,1-dimethylethyl (2S)-2-(3-bromo-2-oxopropyl)-1-piperidinecarboxylate (D2)

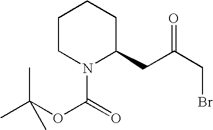

### Preparation (i)

In a 500 ml round-bottom flask under nitrogen at room temperature, 1,1-dimethylethyl (2S)-2-[2-(methyloxy)-2-oxoethyl]-1-piperidinecarboxylate D1 (11.10 g, 43.10 mmol) was dissolved in THF (100 ml) to give a pale yellow solution. This solution was cooled to −78° C. and the Tebbe reagent (104 ml of a 0.5 M solution in toluene, 51.80 mmol) was added dropwise. The thick mixture was diluted with further 70 ml of dry toluene. The resulting brown-orange mixture was stirred at −78° C. for 30 min and then slowly warmed up to room temperature and left under stirring for 2 h. The reaction mixture was charged into a dropping funnel and then added dropwise to a 2 L round-bottom flask containing about 400 ml of an ice-cooled 1 M NaOH aqueous solution. At the end of the quench, the resulting grey suspension was diluted with EtOAc (250 ml) and allowed to stir overnight. The resulting yellow suspension was then filtered over a Gooch funnel and salts were washed with EtOAc (500 ml). Phases were then separated and the organic layer was washed with brine (2×500 ml). The organic phase was dried (Na₂SO₄), filtered and concentrated to give a deep orange oil. The residue was diluted with Et₂O (about 500 ml). Some salts precipitated and the resulting suspension was filtered over a Gooch funnel. The filtrate was concentrated under vacuum to give 12.40 g of 1,1-dimethylethyl (2S)-2-[2-(methyloxy)-2-propen-1-yl]-1-piperidinecarboxylate as an orange-brown crude oil. The material contained some residual salts (the overall recovered amount was higher than the theoretical amount). The material was used without further purification in the next reaction and supposed to be pure at 88.7 wt %. In a 1 L round-bottom flask under nitrogen at room temperature 1,1-dimethylethyl (2S)-2-[2-(methyloxy)-2-propen-1-yl]-1-piperidinecarboxylate (12.40 g, 43.10 mmol) was dissolved in THF (125 ml) and water (35 ml) to give a pale yellow solution. NBS (7.67 g, 43.10 mmol) was then added dissolved in about 100 ml of THF. The resulting grey mixture was stirred at room temperature for 1 h. Additional NBS (1.50 g, 0.2 eq) dissolved in 50 ml of THF was added and the reaction mixture stirred at room temperature for 1 h. The mixture was concentrated under vacuum to remove THF, then was diluted with EtOAc (about 500 ml) and water (200 ml). Phases were separated and the aqueous layer was back-extracted with EtOAc (250 ml). The combined organic layers were dried (Na₂SO₄), filtered and concentrated to give 17.80 g of a brown oil. The material was purified by flash chromatography on silica gel (Biotage 75 L, Cy/EtOAc from 100/0 to 90/10) to give the title compound D2 (6.00 g, 18.70 mmol, 43.5% yield from D1, two steps) as a yellow oil.

UPLC: rt=0.79 min, peaks observed: 342 (M+Na, 100%) and 344 (M+Na, 100%), 264 (M-tBu, 100%) and 266 (M-tBu, 100%). C₁₃H₂₂BrNO₃ requires 319.

¹H NMR (400 MHz, CDCl₃) δ (ppm): 4.72-4.79 (m, 1H), 3.91-4.10 (m, 3H), 2.77-2.97 (m, 3H), 1.49-1.75 (m, 6H), 1.46 (s, 9H).

### Alternative Preparation (ii)

An alternative route to (1,1-dimethylethyl (2S)-2-(3-bromo-2-oxopropyl)-1-piperidinecarboxylate) D2 is the following:

A stirred solution of DIPA (7.84 ml, 56.00 mmol) in THF (70 ml) was cooled to 0° C. and n-BuLi (35.70 ml of a 1.6 M solution in Cy, 57.10 mmol) was added dropwise. To a solution of dibromomethane (3.58 ml, 51.30 mmol) in THF (70 ml) cooled to −90° C. was added dropwise the LDA solution previously prepared. After 5 min stirring, a solution of 1,1-dimethylethyl (2S-2-[2-(methyloxy)-2-oxoethyl]-1-piperidinecarboxylate D1 (6.00 g, 23.30 mmol) in THF (47 ml) was added dropwise to the reaction mixture and then, after 10 min, n-BuLi (22.20 ml of a 1.6 M solution in Cy, 35.50 mmol) was added. After 5 min the resulting mixture was added, via cannula, to a rapidly stirring solution of AcCl (35.00 ml, 492 mmol) in absolute EtOH (230 ml) cooled to −78° C. The reaction mixture was left under stirring and then diluted with Et₂O (400 ml). The mixture was transferred into a separatory funnel and washed with a cold 10% H₂SO₄ aqueous solution (2×100 ml), a 5% NaHCO₃ aqueous solution (100 ml) and brine (100 ml). The organic phase was dried (Na₂SO₄), filtered and the solvent removed under reduced pressure. Purification by flash chromatography on silica gel (Biotage SP1 40 M, DCM) gave the title compound D2 (1.14 g, 3.56 mmol, 15% yield). NMR and MS confirmed the product.

Alternative Preparation (iii)

In a 1 L round-bottom flask titanocene dichloride (60 g, 0.24 mol) was suspended in dry toluene (300 ml) under nitrogen atmosphere and cooled down to 0° C. Methylmagnesium chloride (3 M solution in THF, 180 ml, 0.54 mol) was added dropwise (over 45 min), keeping the internal temperature below 8° C. The resulting mixture was stirred at 0-5° C. for 1.5 h and then transferred (over 30 min) through a siphon in an ice-cooled 6% w/w NH₄Cl aqueous solution (180 ml), keeping the internal temperature below 5° C. The mixture was stirred at 0-5° C. for 1 h. Celite (15 g) was added, the mixture stirred at 10° C. for 15 min and then filtered washing with toluene (20 ml). Phases were separated. The organic layer was washed with water (180 ml) and brine (180 ml), dried (Na₂SO₄), filtered End then distilled down under vacuo to 200 ml. The dimethyltitanocene solution in toluene was charged in a 1 L round-bottom flask under nitrogen atmosphere and 1,1-dimethylethyl (2S)-2-[2-(methyloxy)-2-oxoethyl]-1-piperidinecarboxylate (20 g, 0.078 mol) was ad Led. The resulting mixture was stirred at 90° C. for 3 h. Toluene (500 ml) and iso-octane (50 ml) were added and the mixture filtered through a celite pad to remove inorganic salts. A CU NO filtration (R55S cartridge) was then performed to remove the finest particle size solid. The resulting clear solution was concentrated under vacuo to afford the intermediate 1,1-dimethylethyl (2S)-2-{2-[(methyloxy)methyl]-2-propen-1-yl}-1-piperidinecarboxylate as an orange oil (13.60 g, 0.053 mol, 68% yield). HPLC (walk-up): rt=4.69 min. ¹H-NMR (400 MHz, CDCl₃) δ (ppm): 4.42-4.58 (m, 1H), 3.94-4.08 (m, 1H), 3.88-3.93 (m, 2H), 3.53 (s, 3H), 2.79 (t, 1H), 2.42 (dd, 1H), 2.27 (dd, 1H), 1.50-1.70 (m, 6H), 1.46 (s, 9H).

NBS (8.36 g, 0.047 mol) was added portionwise to a mixture of 1,1-dimethylethyl (2S)-2-{2-[(methyloxy)methyl]-2-propen-1-yl}-1-piperidinecarboxylate (10 g, 0.039 mol) in THF (70 ml) and H₂O (15 ml). The mixture was diluted with TBME (100 ml) and water (50 ml). The aqueous phase was back-extracted with TBME (50 ml). The collected organic phases were washed (twice) with a 4% w/w NaHCO₃ aqueous solution, dried (Na₂SO₄), filtered and evaporated under vacuo. The residual oil was purified by filtration through a silica pad (20 g, toluene/EtOAc 90/10). A further filtration through a silica pad (50 g, toluene/TBME 90/10) afforded the title compound D2 (7.80 g, 0.024 mol, 62% yield).

¹H-NMR (600 MHz, DMSO-d₆) δ (ppm): 4.50-4.64 (m, 1H), 4.35 (s, 2H), 3.70-3.88 (m, 1H), 2.86-3.01 (m, 1H), 2.65-2.82 (m, 2H), 1.42-1.60 (m, 5H), 1.35 (s, 9H), 1.14-1.28 (m, 1H).

### Description 3

### 1,1-dimethylethyl (2S)2-{[7-(trifluoromethyl)imidazo[1,2a]-pyridin-2-yl]methyl}-1-piperidinecarboxylate (D3)

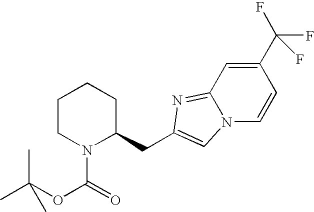

To a solution of 1,1-dimethylethyl (2S)-2-(3-bromo-2-oxopropyl)-1-piperidinecarboxylate D2 (0.30 g, 0.94 mmol) in DMF (2 ml) was added 4-(trifluoromethyl)-2-pyridinamine (0.23 g, 1.41 mmol) and the mixture was stirred at 80° C. for 1.5 h. The reaction mixture was diluted with brine and a saturated NaHCO₃ aqueous solution and then extracted with EtOAc. The residue was purified by flash chromatography on silica gel (Biotage 25M, Cy/EtOAc from 90/10 to 50/50). Collected fractions gave the title compound D3 (0.19 g, 0.50 mmol, 53% yield) as a white solid contaminated with some residual 4-(trifluoromethyl)-2-pyridinamine. UPLC: rt=0.69 min, peak observed: 384 (M+1). C₁₉H₂₄F₃N₃O₂ requires 383.

### Description 4

### 2-[(2S)-2-piperidinylmethyl]-7-(trifluoromethyl)imidazo[1,2-a]pyridine (D4)

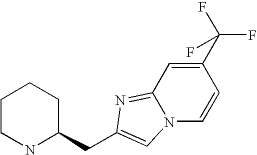

To a solution of 1,1-dimethylethyl (2S)-2-{[7-(trifluoromethyl)imidazo[1,2-a]pyridin-2-yl]methyl}-1-piperidinecarboxylate D3 (0.050 g, 0.13 mmol) in dry DCM (1.50 ml), TFA (0.50 ml) was added and the reaction mixture left under stirring at room temperature for 1 h. Solvent removal afforded a residue that was eluted through a SCX column. Collected fractions gave the title compound D4 (0.035 g, 0.12 mmol, 95% yield) as a colourless oil. UPLC: rt=0.46 min, peak observed: 284 (M+1). C₁₄H₁₆F₃N₃ requires 283.

### Description 5

### 1,1-dimethylethyl (2S)-2-{[6-(trifluoromethyl)imidazo[1,2-a]pyridin-2-yl]methyl}-1-piperidinecarboxylate (D5)

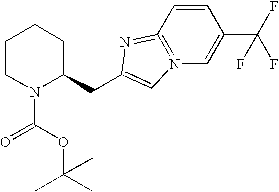

In a 7 ml screw capped vial 1,1-dimethylethyl (2S)-2-(3-bromo-2-oxopropyl)-1-piperidinecarboxylate D2 (0.050 g, 0.16 mmol), DMF (1 ml) and 5-(trifluoromethyl)-2-pyridinamine (0.038 g, 0.23 mmol) were added and the resulting mixture stirred at 80° C. for 13 h. The mixture was diluted with water and extracted with EtOAc to afford 0.068 g of a crude containing the title compound D5 and some residual 5-(trifluoromethyl)-2-pyridinamine. The material was used in the next step without further purification.

HPLC (walk-up): rt=3.85 min. MS: (ES/+) m/z: 384 (M+1). C₁₉H₂₄F₃N₃O₂ requires 383.

### Description 6

### 2-[(2S)-2-piperidinylmethyl]-6-(trifluoromethyl)imidazo[1,2-a]pyridine (D6)

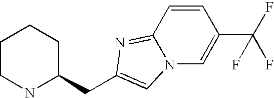

A mixture of 1,1-dimethylethyl (2S)-2-{[6-(trifluoromethyl)imidazo[1,2-a]pyridin-2-yl]methyl}-1-piperidinecarboxylate D5 (0.068 g of a material contaminated with some residual 5-(trifluoromethyl)-2-pyridinamine as reported in Description 5) and DCM (4 ml) was cooled to 0° C. TFA (1 ml) was added dropwise and the reaction mixture left under stirring at room temperature for 3 h. Solvent removal afforded a residue that was eluted through a SCX column. Collected fractions gave 0.070 g of a crude containing the title compound D6 and some residual 5-(trifluoromethyl)-2-pyridinamine. The material was used in the next step without further purification.

HPLC (walk-up): rt=2.29 min. MS: (ES/+) m/z: 284 (M+1). C₁₄H₁₆F₃N₃ requires 283.

### Description 7

### 1,1-dimethylethyl (2S)-2-{[8-(trifluoromethyl)imidazo[1,2-a]pyridin-2-yl]methyl}-1-piperidinecarboxylate (D7)

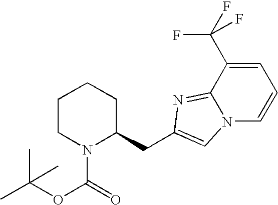

In a 7 ml screw cap vial 1,1-dimethylethyl (2S)-2-(3-bromo-2-oxopropyl)-1-piperidinecarboxylate D2 (0.10 g, 0.31 mmol), DMF (1 ml) and 3-(trifluoromethyl)-2-pyridinamine (0.076 g, 0.47 mmol) were added and the resulting mixture stirred at 80° C. for 13 h. The mixture was eluted through a SCX column. Collected fractions gave 0.15 g of a crude containing the title compound D7, the corresponding free amine and some residual 3-(trifluoromethyl)-2-pyridinamine. The material was used in the next step without further purification. HPLC (walk-up): rt=3.79 min. MS: (ES/+) m/z: 384 (M+1). C₁₉H₂₄F₃N₃O₂ requires 383.

### Description 8

### 2-[(2S)-2-piperidinylmethyl]-8-(trifluoromethyl)imidazo[1,2-a]pyridine (D8)

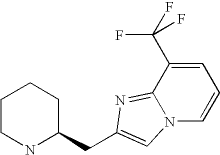

To a solution of 1,1-dimethylethyl (2S)-2-{[8-(trifluoromethyl)imidazo[1,2-a]pyridin-2-yl]methyl}-1-piperidinecarboxylate D7 (0.064 g, 0.17 mmol) in DCM (2.50 ml), TFA (0.50 ml) was added dropwise at 0° C. and the solution was stirred for 1 h. Volatiles were removed under reduced pressure and the residue was eluted through a SCX column. Collected fractions gave the title compound D8 (0.035 g, 0.12 mmol, 74% yield).

LC-MS: rt=0.33 min, peak observed: 284 (M+1). C₁₄H₁₆F₃N₃ requires 283.

### Description 9

### 1,1-dimethylethyl (2S)-2-[(6,8-dichloroimidazo[1,2-a]pyridin-2-yl)methyl]-1-piperidinecarboxylate (D9)

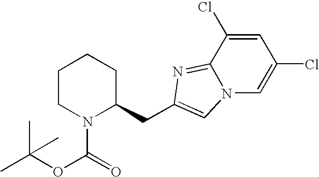

In a 7 ml screw cap vial 1,1-dimethylethyl (2S)-2-(3-bromo-2-oxopropyl)-1-piperidinecarboxylate D2 (0.52 g, 0.16 mmol), DMF (3.80 ml) and 3,5-dichloro-2-pyridinamine (0.040 g, 0.25 mmol) were added and the resulting mixture stirred at 80° C. for 3 h. The mixture was diluted with brine and extracted with EtOAc. The organic layer was washed with brine/ice, dried (Na₂SO₄), filtered and evaporated under vacuum to give 0.10 g of a crude containing the title compound D9. The material was used in the next step without further purification. MS: (ES/+) m/z: 384 (M+1, 100%) and 386 (M+1, 66%). C₁₈H₂₃Cl₂N₃O₂ requires 383.

### Description 10

### 6,8-dichloro-2-[(2S)-2-piperidinylmethyl]imidazo[1,2-a]pyridine (D10)

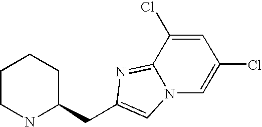

A mixture of 1,1-dimethylethyl (2S)-2-[(6,8-dichloroimidazo[1,2-a]pyridin-2-yl)methyl]-1-piperidinecarboxylate D9 (0.10 g of the crude material obtained in Description 9) and DCM (4 ml) was cooled to 0° C. TFA (1 ml) was added dropwise and the reaction mixture left under stirring at room temperature for 1 h. Solvent removal afforded a residue that was eluted through a SCX column. Collected fractions gave 0.051 g of a crude yellow oil containing the title compound D10. The material was used without further purification in the next step.

MS: (ES/+) m/z: 284 (M+1, 100%) and 286 (M+1, 66%). C₁₃H₁₅Cl₂N₃ requires 283.

### Description 11

### 1,1-dimethylethyl (2S)-2-[(8-methylimidazo[1,2-a]pyridin-2-yl)methyl]-1-piperidinecarboxylate (D11)

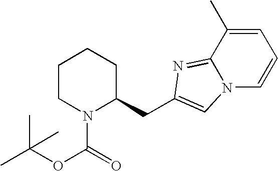

In a 50 ml round-bottom flask at room temperature under nitrogen, 1,1-dimethylethyl (2S)-2-(3-bromo-2-oxopropyl)-1-piperidinecarboxylate D2 (0.12 g, 0.375 mmol) was dissolved in DMF (2 ml) to give a pale yellow solution. 3-Methyl-2-pyridinamine (0.0608 g, 0.562 mmol) was then added and the resulting solution heated at 80° C. for 45 min. The mixture was allowed to cool down to room temperature and was diluted with brine (5 ml) and Et₂O (2 ml). Phases were separated and the aqueous layer extracted with Et₂O (3×3 ml). The combined organic layers were dried (Na₂SO₄), filtered and concentrated to give 0.12 g of a crude pale yellow oil containing the title compound D11. The material was used without further purification in the next step. UPLC: rt=0.54 min, peak observed: 330 (M+1). C₁₉H₂₇N₃O₂ requires 329.

### Description 12

### 8-methyl-2-[(2S)-2-piperidinylmethyl]imidazo[1,2-a]pyridine (D12)

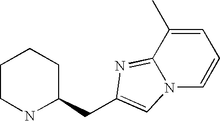

In a 100 ml pear flask 1,1-dimethylethyl (2S)-2-[(8-methylimidazo[1,2-a]pyridin-2-yl)methyl]-1-piperidinecarboxylate D11 (1.70 g, 5.16 mmol) was dissolved in DCM (30 ml) to give a yellow solution that was cooled to 0° C. TFA (5 ml) was added dropwise and the resulting mixture left under stirring overnight. The mixture was evaporated under vacuum and the crude dark oil was eluted through a SCX column. Collected fractions gave the title compound D12 (1.05 g, 4.39 mmol, 85% yield) as an oil. HPLC (walk-up): rt=1.85 min. UPLC: rt=0.31 min, peak observed: 230 (M+1). C₁₄H₁₉N₃ requires 229.

¹H NMR (400 MHz, CDCl₃) δ (ppm): 7.94 (d, 1H), 7.41 (s, 1H), 6.94 (d, 1H), 6.66 (t, 1H), 2.89-3.06 (m, 1H), 2.93-3.01 (m, 2H), 2.71-2.79 (m, 1H), 2.58-2.67 (m, 4H), 1.85-1.95 (bs, NH), 1.75-1.84 (m, 2H), 1.58-1.64 (m, 1H), 1.22-1.55 (m, 3H).

### Description 13

### 6,8-difluoro-2-[(2S)-2-piperidinylmethyl]imidazo [1,2-a]pyridine (D13)

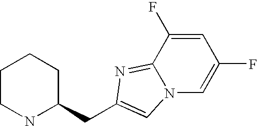

To a solution of 1,1-dimethylethyl (2S)-2-(3-bromo-2-oxopropyl)-1-piperidinecarboxylate D2 (0.29 mmol) in DMF (1 ml), 3,5-difluoro-2-pyridinamine (0.056 g, 0.43 mmol) was added and the mixture was stirred at 80° C. for 2.5 h. The reaction mixture was eluted through a SCX column. Collected fractions gave 0.066 g of an oil containing a mixture of the final compound, the corresponding N-Boc derivative and some residual 3,5-difluoro-2-pyridinamine. [N-Boc derivative data: MS: (ES/+) m/z: 352 (M+1). C₁₈H₂₃F₂N₃O₂ requires 351. UPLC: rt=0.69 min, peak observed: 352 (M+1)]. The crude was dissolved in DCM (2.50 ml) and the resulting solution cooled to 0° C. TFA (0.50 ml) was added dropwise, the reaction left under stirring for 1 h and then eluted through a SCX column. Collected fractions gave the title compound D13 (0.041 g, 0.16 mmol, 55% yield from D2, two steps). LC-MS: rt=0.32 min, peak observed: 252 (M+1). C₁₃H₁₅F₂N₃ requires 251.

### Description 14

### 6-fluoro-2-[(2S)-2-piperidinylmethyl]imidazo[1,2-a]pyridine (D14)

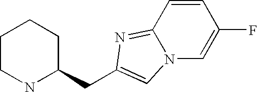

To a solution of 1,1-dimethylethyl (2S)-2-(3-bromo-2-oxopropyl)-1-piperidinecarboxylate D2 (0.10 g, 0.31 mmol) in DMF (1 ml), 5-fluoro-2-pyridinamine (0.053 g, 0.47 mmol) was added and the mixture heated at 80° C. for 2 h. The reaction mixture was eluted through a SCX column. Collected fractions gave 0.075 g of an oil containing a mixture of the final compound and the corresponding N-Boc protected derivative. [N-Boc derivative data:

MS: (ES/+) m/z: 334 (M+1). C₁₈H₂₄FN₃O₂ requires 333]. The crude was dissolved in DCM (2.50 ml) and the resulting solution cooled to 0° C. TFA (0.50 ml) was added dropwise, the reaction left under stirring for 1 h and then eluted through a SCX column. Collected fractions gave the title compound D14 (0.051 g, 0.22 mmol, 71% yield from D2, two steps).

LC-MS: rt=0.24 min, peak observed: 234 (M+1). C₁₃H₁₆FN₃ requires 233.

### Description 15

### 2-[(2S)-2-piperidinylmethyl]imidazo[1,2-a]pyridine-7-carbonitrile (D15)

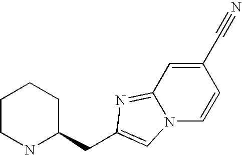

To a solution of 1,1-dimethylethyl (2S)-2-(3-bromo-2-oxopropyl)-1-piperidinecarboxylate D2 (0.27 mmol) in DMF (1 ml), 2-amino-4-pyridinecarbonitrile (0.032 g, 0.27 mmol) was added and the mixture heated at 80° C. for 2.5 h. The reaction was eluted through a SCX column. Collected fractions gave 0.049 g of an oil containing a mixture of the final compound, the corresponding N-Boc protected derivative and some residual 2-amino-4-pyridinecarbonitrile. [N-Boc derivative data: HPLC: rt=0.65 min, peak observed: 341 (M+1). C₁₉H₂₄N₄O₂ requires 340]. The crude was dissolved in DCM (2.50 ml) and the resulting solution cooled to 0° C. TFA (0.50 ml) was added dropwise, the reaction left under stirring for 1 h and then eluted through a SCX column. Collected fractions gave the title compound D15 (0.041 g, 0.17 mmol, 63% yield from D2, two steps) contaminated with some residual 2-amino-4-pyridinecarbonitrile.

UPLC: rt=0.38 min, peak observed: 241 (M+1). C₁₄H₁₆N₄ requires 240.

### Description 16

### 6-bromo-7,8-dimethyl-2-[(2S)-2-piperidinylmethyl]imidazo[1,2-a]pyridine (D16)

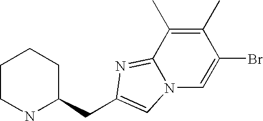

To a solution of 1,1-dimethylethyl (2S)-2-(3-bromo-2-oxopropyl)-1-piperidinecarboxylate D2 (0.13 g, 0.39 mmol) in DMF (1 ml), 5-bromo-3,4-dimethyl-2-pyridinamine (0.12 g, 0.59 mmol) was added and the mixture heated at 80° C. for 2 h. The reaction was eluted through a SCX column. Collected fractions gave 0.13 g of an oil containing a mixture of the final compound, the corresponding N-Boc protected derivative and some residual 5-bromo-3,4-dimethyl-2-pyridinamine. [N-Boc derivative data: MS: (ES/+) m/z: 422 (M+1, 100%) and 424 (M+1, 100%). C₂₀H₂₈BrN₃O₂ requires 421]. The crude was dissolved in DCM (2.50 ml) and the resulting solution cooled to 0° C. TFA (0.50 ml) was added dropwise, the reaction left under stirring for 1 h and then eluted through a SCX column. Collected fractions gave the title compound D16 (0.090 g, 0.28 mmol, 72% yield from D2, two steps). MS: (ES/+) m/z: 322 (M+1, 100%) and 324 (M+1, 100%). C₁₅H₂₀BrN₃ requires 321.

### Description 17

### 2-[(2S)-2-piperidinylmethyl]-5-(trifluoromethyl)imidazo[1,2-a]pyridine (D17)

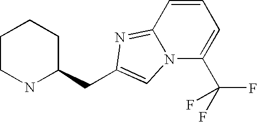

To a solution of 1,1-dimethylethyl (2S)-2-(3-bromo-2-oxopropyl)-1-piperidinecarboxylate D2 (0.10 g, 0.32 mmol) in DMF (1 ml), 6-(trifluoromethyl)-2-pyridinamine (0.077 g, 0.48 mmol) was added and the mixture heated at 80° C. for 3 h. The reaction was eluted through a SCX column. Collected fractions gave 0.070 g of an oil containing the N-Boc protected derivative contaminated with some residual 6-(trifluoromethyl)-2-pyridinamine. [N-Boc derivative data: MS: (ES/+) m/z: 384 (M+1). C₁₉H₂₄F₃N₃O₂ requires 383]. The crude was dissolved in DCM (4 ml) and the resulting solution cooled to 0° C. TFA (1 ml) was added dropwise, the reaction left under stirring for 1 h and then eluted through a SCX column. Collected fractions gave the title compound D17 (0.060 g, 0.21 mmol, 66% yield from D2, two steps). MS: (ES/+) m/z: 284 (M+1). C₁₄H₁₆F₃N₃ requires 283.

### Description 18

### 6-bromo-5-methyl-2-[(2S)-2-piperidinylmethyl]imidazo[1,2-a]pyridine (D18)

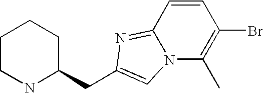

To a solution of 1,1-dimethylethyl (2S)-2-(3-bromo-2-oxopropyl)-1-piperidinecarboxylate D2 (0.10 g, 0.31 mmol) in DMF (1 ml), 5-bromo-6-methyl-2-pyridinamine (0.088 g, 0.47 mmol) was added and the mixture heated at 80° C. for 2 h. The reaction was eluted through a SCX column. Collected fractions gave 0.12 g of an oil containing the final compound, the corresponding N-Boc protected derivative and some residual 5-bromo-6-methyl-2-pyridinamine. [N-Boc derivative data: MS: (ES/+) m/z: 408 (M+1, 100%), 410 (M+1, 100%). C₁₉H₂₆BrN₃O₂ requires 407]. The crude was dissolved in DCM (2.50 ml) and the resulting solution cooled to 0° C. TFA (0.50 ml) was added dropwise, the reaction left under stirring for 1 h and then eluted through a SCX column. Collected fractions gave the title compound D18 contaminated with some residual 5-bromo-6-methyl-2-pyridinamine (0.087 g, 0.28 mmol, 90% yield from D2, two steps). MS: (ES/+) m/z: 308 (M+1, 100%) and 310 (M+1, 100%) C₁₄H₁₈BrN₃ requires 307.

### Description 19

### 1,1-dimethylethyl (2S)-2-[(8-fluoroimidazo[1,2-a]pyridin-2-yl)methyl]-1-piperidinecarboxylate (D19)

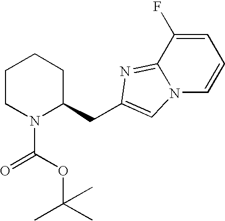

1,1-D1methylethyl (2S)-2-(3-bromo-2-oxopropyl)-1-piperidinecarboxylate D2 (42.80 g, 134 mmol) and 3-fluoro-2-pyridinamine (14.98 g, 134 mmol) were dissolved in dry DMF (240 ml) and the resulting solution was stirred at 80° C. for 4 h. The reaction mixture was cooled to 25° C. and was diluted with NaHCO₃ sat aqueous solution/water 1/1 (470 ml) and extracted with Et₂O (3×941 ml). The organic layers were combined, dried (Na₂SO₄) and the solvent removed under reduced pressure. The residue was purified by flash chromatography on silica gel (Biotage 75 L, Cy/EtOAc/MeOH from 80/20/2.5 to 80/20/10) to afford 25.70 g of the title compound D19 contaminated with 3-fluoro-2-pyridinamine (25% from NMR analysis). The material was dissolved in DCM (650 ml). Ps-TsCl [38 g, 74.90 mmol (resin capacity 1.97 mmol/g)] and then DMAP (3 g, 24.56 mmol) were added. The resulting mixture was stirred at room temperature under Argon atmosphere overnight and filtered. The filtrate was dried (Na₂SO₄), the solvent removed under vacuum and the crude purified by flash chromatography on silica gel (Biotage 75 L, Cy/EtOAc/MeOH from 80/20/2 to 80/20/5) to afford the title compound D19 (23.56 g, 70.70 mmol, 53% yield from D2) contaminated with some residual 3-fluoro-2-pyridinamine (14% from NMR analysis).

1H NMR (400 MHz, CDCl₃) δ (ppm): 7.86 (d, 1H), 7.40-7.57 (bs, 1H), 6.79-6.90 (m, 1H), 6.60-6.71 (m, 1H), 4.63-4.77 (m, 1H), 3.97-4.16 (m, 1H), 3.18-3.34 (m, 1H), 2.86-3.03 (m, 2H), 1.33-1.81 (m, 6H), 1.13-1.37 (bs, 9H).

### Description 20

### 7-(methyloxy)-2-[(2S)-2-piperidinylmethyl]imidazo[1,2-a]pyridine (D20)

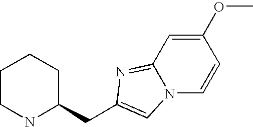

To a solution of 1,1-dimethylethyl (2S)-2-(3-bromo-2-oxopropyl)-1-piperidinecarboxylate D2 (0.11 g, 0.27 mmol) in DMF (1 ml) was added 4-(methyloxy)-2-pyridinamine (0.033 g, 0.27 mmol) and the mixture was stirred at 80° C. for 2.5 h. The reaction mixture was eluted through a SCX column. Collected fractions gave 0.058 g of an oil containing a mixture of the title compound, the corresponding N-Boc protected derivative and some residual 4-(methyloxy)-2-pyridinamine. [N-Boc derivative data. LC-MS: rt=1.44 min, peak observed m/z=346 (M+1). C₁₉H₂₇N₃O₃ requires 345]. The crude was dissolved in DCM (2.50 ml) and the resulting solution cooled to 0° C. TFA (0.50 ml) was added dropwise, the reaction left under stirring for 1 h and then eluted through a SCX column. Collected fractions gave the title compound D20 (0.050 g) contaminated with 4-(methyloxy)-2-pyridinamine. The material was used without further purification in the next step.

UPLC: rt=0.43 min, peak observed: 246 (M+1). C₁₄H₁₉N₃O requires 245.

### Description 21

### 2-[(2S)-2-piperidinylmethyl]imidazo[1,2-a]pyridine-8-carbonitrile (D21)

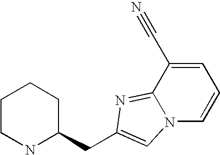

To a solution of 1,1-dimethylethyl (2S)-2-(3-bromo-2-oxopropyl)-1-piperidinecarboxylate D2 (0.11 g, 0.275 mmol) in DMF (1 ml) was added 2-amino-3-pyridinecarbonitrile (0.0491 g, 0.412 mmol) and the mixture was stirred at 80° C. for 2.5 h. The reaction mixture was eluted through a SCX column eluted with ammonia in methanol. Collected fractions gave 0.054 g of an oil containing the title compound, the corresponding N-Boc protected derivative and some residual 2-amino-3-pyridinecarbonitrile. [N-Boc derivative data. HPLC: rt=0.68 min, peak observed: 341 (M+1). C₁₉H₂₄N₄O₂ requires 340]. The crude was dissolved in DCM (1 ml) and the resulting solution cooled to 0° C. TFA (0.20 ml) was added dropwise, the reaction left under stirring for 1 h and then eluted through a SCX column. Collected fractions gave the title compound D21 (0.050 g) contaminated with 2-amino-3-pyridinecarbonitrile. The material was used without further purification in the next step.

UPLC: rt=0.38 min, peak observed: 241 (M+1). C₁₄H₁₆N₄ requires 240.

### Description 22

### 5-fluoro-2-[(2S)-2-piperidinylmethyl]imidazo[2-a]pyridine (D22)

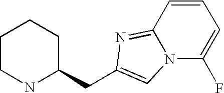

To a solution of 1,1-dimethylethyl (2S)-2-(3-bromo-2-oxopropyl)-1-piperidinecarboxylate D2 (0.11 g, 0.26 mmol) in DMF (1 ml) was added 6-fluoro-2-pyridinamine (0.029 g, 0.26 mmol) and the mixture was stirred at 80° C. for 2.5 h. The reaction mixture was eluted through a SCX column. Collected fractions gave 0.032 g of an oil containing a mixture of the title compound, the corresponding N-Boc protected derivative and some residual 6-fluoro-2-pyridinamine. [N-Boc derivative data. LC-MS: rt=1.54 min, peak observed: 334 (M+1). C₁₈H₂₄FN₃O₂ requires 333]. The crude was dissolved in DCM (2.50 ml) and the resulting solution cooled to 0° C. TFA (0.50 ml) was added dropwise, the reaction left under stirring for 1 h and then eluted through a SCX column. Collected fractions gave the title compound D22 (0.020 g) contaminated with 6-fluoro-2-pyridinamine. The material was used without further purification in the next step.

HPLC (walk-up): rt=1.50 min. MS: (ES/+) m/z: 234 (M+1). C₁₃H₁₆FN₃ requires 233.

### Description 23

### 1,1-dimethylethyl (2S)-2-(imidazo[1,2-a]pyridin-2-ylmethyl)-1-piperidinecarboxylate (D23)

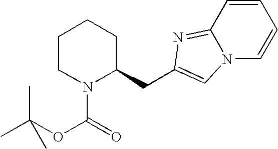

To a solution of 1,1-dimethylethyl (2S)-2-(3-bromo-2-oxopropyl)-1-piperidinecarboxylate D2 (0.269 g, 0.84 mmol) in DMF (2.50 ml) was added 2-pyridinamine (0.095 g, 1.008 mmol) and the mixture was stirred at 60° C. for 2 h. The reaction mixture was diluted with brine (5 ml) and extracted with EtOAc (2×5 ml). The combined organic layers were washed with brine/ice (6×5 ml), dried (Na₂SO₄) and the solvent removed under reduced pressure. The residue was purified by flash chromatography on silica gel (Biotage SP1 12M, DCM/MeOH/TEA 98/2/0.5) to afford the title compound D23 (0.13 g, 0.412 mmol, 49.1% yield). HPLC: rt=0.51 min, peak observed: 316 (M+1). C₁₈H₂₅N₃O₂ requires 315.

¹H NMR [the product is present as a mixture of conformers (ratio ca. 85/15) and the assignment refers to the major component] (400 MHz, CDCl₃) δ (ppm): 8.03 (dt, 1H), 7.54 (d, 1H), 7.37-7.44 (m, 1H), 7.10-7.16 (m, 1H), 6.73 (td, 1H), 4.62-4.71 (m, 1H), 4.00-4.11 (m, 1H), 3.19 (dd, 1H), 2.90-3.02 (m, 2H), 1.62-1.76 (m, 6H), 1.26 (bs, 9H).

### Description 24

### 1,1-dimethylethyl (2S)-2-[(3-iodoimidazo[1,2-a]pyridin-2-yl)methyl]-1-piperidinecarboxylate (D24)

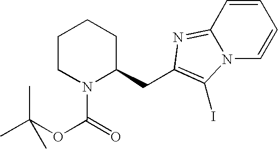

To a solution of 1,1-dimethylethyl 2-(imidazo[1,2-a]pyridin-2-ylmethyl)-1-piperidinecarboxylate D23 (0.13 g, 0.412 mmol) in DCM (50 ml), 12 (13 ml of a 1 M DCM solution, 13.00 mmol) was added dropwise at room temperature and the resulting mixture was stirred for 3 h. A 5% NaHSO₃ aqueous solution (20 ml) was added, followed by KF (20 ml of a 1 M MeOH solution) and the mixture was vigorously stirred for 10 min. The organic phase was separated, dried (Na₂SO₄), filtered and concentrated to give the title compound D24 (0.172 g, 0.378 mmol, 92% yield). ¹H NMR (400 MHz, CDCl₃) δ (ppm): 8.12 (d, 1H), 7.66 (bd, 1H), 7.27-7.33 (m, 1H), 6.97 (t, 1H), 4.67-4.75 (m, 1H), 4.06-4.14 (m, 1H), 3.19 (dd, 1H), 3.13 (dd, 1H), 2.99 (dd, 1H), 1.63-1.78 (m, 6H), 1.19 (bs, 9H).

### Description 25

### 3-iodo-2-[(2S)-2-piperidinylmethyl]imidazo[1,2-a]pyridine (D25)

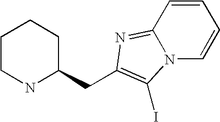

To a solution of 1,1-dimethylethyl (2S)-2-[(3-iodoimidazo[1,2-a]pyridin-2-yl)methyl]-1-piperidinecarboxylate D24 (0.020 g, 0.045 mmol) in DCM (1 ml), TFA (0.20 ml) was added dropwise at 0° C. and the solution was stirred for 1 h. Volatiles were removed under reduced pressure and the residue was eluted through a SCX column. Collected fractions gave the title compound D25 (0.014 g, 0.041 mmol, 91% yield) as a brown oil.

UPLC: rt=0.40 min, peak observed: 342 (M+1). C₁₃H₁₆1N₃ requires 341.

### Description 26

### 1,1-dimethylethyl (2S)-2-[(3-methylimidazo[1,2-a]pyridin-2-yl)methyl]-1-piperidinecarboxylate (D26)

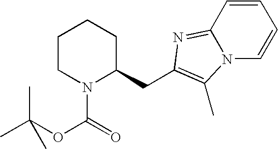

To a mixture of 1,1-dimethylethyl (2S)-2-[(3-iodoimidazo[1,2-a]pyridin-2-yl)methyl]-1-piperidinecarboxylate D24 (0.020 g, 0.045 mmol) and palladium-tetrakis(triphenylphosphine) (0.00262 g, 0.002266 mmol) in DME (0.36 ml) was added methylboronic acid (0.0047 g, 0.068 mmol) followed by the addition of NaOH (0.00363 g, 0.091 mmol) in water (0.18 ml). The resulting mixture was stirred at 90° C. for 72 h. The reaction mixture was poured into water (2 ml) and extracted with DCM (3×2 ml). The organic phases were collected, dried (Na₂SO₄), filtered and the solvent evaporated under vacuum. The yellow residue was purified by flash chromatography on silica gel (Biotage 25M, DCM/MeOH 99/1). Collected fractions gave the title compound D26 (0.008 g, 0.024 mmol, 53.6% yield). UPLC: rt=0.55 min, peak observed: 330 (M+1). C₁₉H₂₇N₃O₂ requires 329.

### Description 27

### 3-methyl-2-[(2S)-2-piperidinylmethyl]imidazo[1,2-a]pyridine (D27)

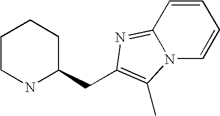

To a solution of 1,1-dimethylethyl (2S)-2-[(3-methylimidazo[1,2-a]pyridin-2-yl)methyl]-1-piperidinecarboxylate D26 (0.008 g, 0.024 mmol) in DCM (1 ml), TFA (0.20 ml) was added dropwise at 0° C. and the solution was stirred for 1 h. Volatiles were removed under reduced pressure and the residue eluted through a SCX column. Collected fractions gave the title compound D27 (0.005 g, 0.022 mmol, 90% yield). HPLC (walk-up): rt=1.62 min.

MS: (ES/+) m/z: 230 (M+1). C₁₄H₁₉N₃ requires 229.

### Description 28

### 3-chloro-2-[(2S)-2-piperidinylmethyl]imidazo[1,2-a]pyridine (D28)

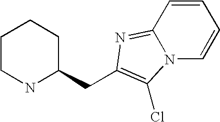

1,1-Dimethylethyl (2S)-2-(imidazo[1,2-a]pyridin-2-ylmethyl)-1-piperidinecarboxylate D23 (0.020 g, 0.063 mmol) was dissolved in DCM (1 ml) and then NCS (0.009 g, 0.070 mmol) was added. The reaction was stirred at room temperature for 2 h. The solvent was removed under reduced pressure to give the N-Boc protected compound [N-Boc derivative data. UPLC: rt=0.66 min, peak observed: 350 (M+1). C₁₈H₂₄ClN₃O₂ requires 349]. The N-Boc derivative (0.063 mmol, supposed quantitative yield) was dissolved in DCM (1 ml), TFA (0.50 ml) was added and the reaction stirred for 2 h. Volatiles were removed under vacuum and the resulting crude eluted through a SCX column. Collected fractions gave the title compound D28 (0.015 g, 0.060 mmol, 95% yield from D23, two steps). HPLC: rt=0.40 min. peaks observed: 250 (M+1, 100%) and 252 (M+1, 33%). C₁₃H₁₆ClN₃ requires 249.

### Description 29

### 1,1-dimethylethyl (2S)-2-{[3-chloro-7-(trifluoromethyl)imidazo[1,2-a]pyridin-2-yl]methyl}-1-piperidinecarboxylate (D29)

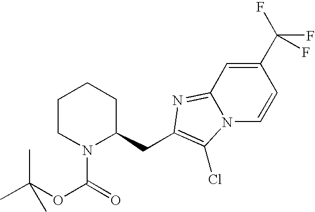

To a solution of 1,1-dimethylethyl (2S)-2-{[7-(trifluoromethyl)imidazo[1,2-a]pyridin-2-yl]methyl}-1-piperidinecarboxylate D3 (0.090 g, 0.24 mmol) in DCM (3 ml) was added NCS (0.031 g, 0.24 mmol) and the reaction mixture was stirred at room temperature for 3 h. The solvent was evaporated and the residue was purified by flash chromatography on silica gel (Biotage 12M, Cy/EtOAc from 100/0 to 70/30). Collected fractions gave the title compound D29 (0.090 g, 0.22 mmol, 92% yield) as a white solid. UPLC: rt=0.90 min, peaks observed: 418 (M+1, 100%) and 420 (M+1, 33%). C₁₉H₂₃ClF₃N₃O₂ requires 417.

### Description 30

### 3-chloro-2-[(2S)-2-piperidinylmethyl]-7-(trifluoromethyl)imidazo[1,2-a]pyridine (D30)

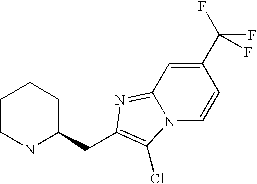

To a solution of 1,1-dimethylethyl (2S)-2-{[3-chloro-7-(trifluoromethyl)imidazo[1,2-a]pyridin-2-yl]methyl}-1-piperidinecarboxylate D29 (0.090 g, 0.22 mmol) in dry DCM (1.50 ml), TFA (0.50 ml) was added and the reaction mixture was stirred at room temperature for 1 h. The solvent was evaporated and the residue eluted through a SCX column. Collected fractions gave the title compound D30 (0.067 g, 0.21 mmol, 98% yield) as a colourless oil. UPLC: rt=0.49 min, peaks observed: 318 (M+1, 100%) and 320 (M+1, 33%). C₁₄H₁₅ClF₃N₃ requires 317.

### Description 31

### 3-fluoro-8-methyl-2-[(2S)-2-piperidinylmethyl]imidazo[1,2-a]pyridine (D31)

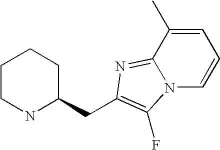

To a solution of 1,1-dimethylethyl (2S)-2-[(8-methylimidazo[1,2-a]pyridin-2-yl)methyl]-1-piperidinecarboxylate D11 (0.165 g, 0.507 mmol) in anhydrous acetonitrile (5 ml), Selectfluor™ (0.090 g, 0.253 mmol) was added at −30° C. The resulting reaction mixture was gradually warmed up to −20° C. and left under stirring for 3 h. The mixture was then diluted with DCM (10 ml) and washed with a 5% aqueous NaHCO₃ solution (2×12 ml). The organic layer was separated through a phase separator tube and evaporated. The residue was purified by flash chromatography on silica gel (Biotage SP4 25M, Cy/EtOAc 80/20). Collected fractions gave the N-Boc protected compound (0.026 g of a slightly contaminated material that was used without further purification in the next step). [N-Boc derivative data: UPLC: rt=0.63 min, peak observed: 348 (M+1). C₁₉H₂₆FN₃O₂ requires 347].

To a solution of the crude N-Boc derivative (0.026 g, 0.075 mmol) in DCM (1 ml), TFA (0.20 ml) was added at 0° C. and the reaction mixture was stirred for 1 h. The solvent was evaporated and the residue eluted through a SCX column. Collected fractions gave the title compound D31 (0.014 g, 0.057 mmol, 12% yield from D11, two steps) as a yellow oil. UPLC: rt=0.38 min, peak observed: 248 (M+1). C₁₄H₁₈FN₃ requires 247.

### Description 32

### 1,1-dimethylethyl (2S)-2-[(3-chloro-6-fluoroimidazo[1,2-a]pyridin-2-yl)methyl]-1-piperidinecarboxylate (D32)

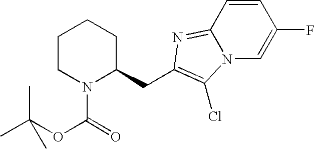

To a solution of 1,1-dimethylethyl (2S)-2-(3-bromo-2-oxopropyl)-1-piperidinecarboxylate D2 (0.11 g, 0.34 mmol) in DMF (1 ml), 5-fluoro-2-pyridinamine (0.058 g, 0.52 mmol) was added and the reaction was stirred for 1.5 h at 80° C. The reaction was diluted with brine and a saturated NaHCO₃ aqueous solution and extracted with EtOAc. The organic layer was dried (Na₂SO₄), filtered and evaporated. The residue was dissolved in dry DCM (2 ml) and NCS (0.046 g, 0.34 mmol) was added. The reaction mixture was stirred for 2 h at room temperature. The solvent was evaporated and the residue purified by flash chromatography on silica gel (Biotage 25M, Cy/EtOAc from 100/0 to 50/50). Collected fractions gave the title compound D32 (0.060 g, 0.16 mmol, 47% yield from D2, two steps) as a pale yellow oil. UPLC: rt=0.80 min, peaks observed: 368 (M+1, 100%) and 370 (M+1, 33%). C₁₈H₂₃ClFN₃O₂ requires 367.

### Description 33

### 3-chloro-6-fluoro-2-[(2S)-2-piperidinylmethyl]imidazo[1,2-a]pyridine (D33)

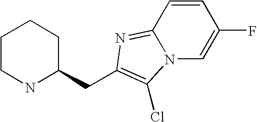

To a solution of 1,1-dimethylethyl (2S)-2-[(3-chloro-6-fluoroimidazo[1,2-a]pyridin-2-yl)methyl]-1-piperidinecarboxylate D32 (0.060 g, 0.16 mmol) in dry DCM (2 ml), TFA (0.50 ml) was added and the reaction mixture stirred for 1 h at room temperature. The solvent was evaporated and the residue eluted through a SCX column. Collected fractions gave the title compound D33 (0.043 g, 0.16 mmol, 98% yield) as a colourless oil. UPLC: rt=0.45 min, peaks observed: 268 (M+1, 100%) and 270 (M+1, 33%). C₁₃H₁₅ClFN₃ requires 267.

### Description 34

### 3-(methyloxy)-2-pyridinamine (D34)

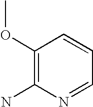

To a stirred solution of 3-(methyloxy)-2-nitropyridine (1.00 g, 6.49 mmol) in EtOH (13 ml), a 2 M HCl aqueous solution (1.34 ml, 2.68 mmol) and iron (2.44 g, 43.70 mmol) were added at 0° C. The resulting mixture was stirred for 2.5 h at room temperature. Celite (2.40 g) was added. The mixture was then filtered through a celite pad and evaporated to give a dark oil that was eluted through a SCX column. Collected fractions gave the title compound D34 (0.50 g, 3.62 mmol, 56% yield) as a dark green solid. ¹H NMR (400 MHz, DMSO-d₆) δ (ppm): 7.49 (dd, 1H), 6.99 (d, 1H), 6.49 (dd, 1H), 5.57-5.63 (bs, 2H), 3.76 (s, 3H).

### Description 35

### 8-(methyloxy)-2-[(2S)-2-piperidinylmethyl]imidazo[1,2-a]pyridine (D35)

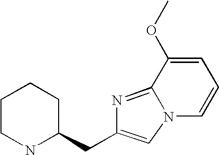

To a solution of 1,1-dimethylethyl (2S)-2-(3-bromo-2-oxopropyl)-1-piperidinecarboxylate D2 (0.12 g, 0.38 mmol) in DMF (1 ml), 3-(methyloxy)-2-pyridinamine D34 (0.056 g, 0.45 mmol) was added and the mixture was stirred at 80° C. for 1 h. The crude was eluted through a SCX column. Collected fractions gave a material containing the desired N-Boc protected compound (0.080 g) slightly contaminated with some residual 3-(methyloxy)-2-pyridinamine. The material was used without further purification in the next step.

[N-Boc derivative data. UPLC: rt=0.56 min, peak observed: 346 (M+1). C₁₉H₂₇N₃O₃ requires 345].

The crude containing the N-Boc derivative (0.080 g) was dissolved in DCM (1 ml) and TFA (1 ml) was added at 0° C. The reaction mixture was left under stirring for 2 h and then eluted through a SCX column. Collected fractions gave the title compound D35 (0.055 g, 0.22 mmol, 58% yield from D2, two steps) contaminated with some residual 3-(methyloxy)-2-pyridinamine. UPLC: rt=0.31 min, peak observed: 246 (M+1). C₁₄H₁₉N₃O requires 245.

### Description 36

### 1,1-dimethylethyl (2S)-2-{[7-(methyloxy)imidazo[1,2-a]pyridin-2-yl]methyl}-1-piperidinecarboxylate (D36)

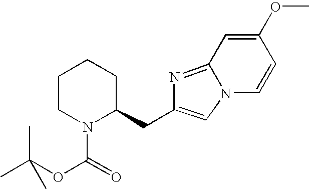

To a solution of 1,1-dimethylethyl (2S)-2-(3-bromo-2-oxopropyl)-1-piperidinecarboxylate D2 (0.30 g, 0.94 mmol) in DMF (2 ml) was added 4-(methyloxy)-2-pyridinamine (0.12 g, 0.94 mmol) and the reaction was stirred for 3 h at 60° C. DMF was removed under vacuum and the resulting crude product purified by flash chromatography on silica gel (Biotage 25M, EtOAc). Collected fraction gave the title compound D36 (0.11 g, 0.30 mmol, 32% yield). UPLC: rt=0.55 min, peak observed: 346 (M+1). C₁₉H₂₇N₃O₃ requires 345.

### Description 37

### 3-chloro-7-(methyloxy)-2-[(2S)-2-piperidinylmethyl]imidazo[1,2-a]pyridine (D37)

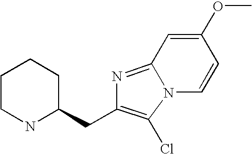

1,1-dimethylethyl (2S)-2-{[7-(methyloxy)imidazo[1,2-a]pyridin-2-yl]methyl}-1-piperidinecarboxylate D36 (0.11 g, 0.30 mmol) was dissolved in DCM (1 ml), then NCS (0.041 g, 0.30 mmol) was added and the mixture stirred for 3 h. DCM (1 ml) was added and the organic phase washed with a saturated NaHCO₃ aqueous solution (1 ml). The biphasic system was filtered through a phase separator tube and the organic phase concentrated to give 0.023 g of a crude material containing the intermediate N-Boc protected compound. [N-Boc derivative data: UPLC: rt=0.62 min, peaks observed: 380 (M+1, 100%) and 382 (M+1, 33%). C₁₉H₂₆ClN₃O₃ requires 379]. The material was dissolved in DCM (1 ml), then TFA (0.003 ml) was added and the reaction mixture stirred at room temperature for 1.5 h. Volatiles were removed and the residue eluted through a SCX column. Collected fractions gave 0.017 g of an impure material containing the title compound D37. The material was used without further purification in the next step. UPLC: rt=0.39 min, peak observed: 280 (M+1). C₁₄H₁₈ClN₃O requires 279.

### Description 38

### 2-chloro-5-fluoro-3-methylpyridine (D38)

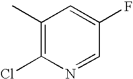

To a −20° C. cooled solution of (2-chloro-5-fluoro-3-pyridinyl)methanol (3.086 g, 19.10 mmol) and TEA (5.32 ml, 38.20 mmol) in anhydrous DCM (180 ml), MsCl (2.233 ml, 28.70 mmol) was added dropwise and the resulting reaction mixture stirred at 0° C. for 30 min. Volatiles were evaporated under reduced pressure to afford the desired mesylate (4.53 g) that was used in the next step without further purification. [Mesylate data: UPLC: rt=0.57 min, peaks observed: 240 (M+1, 100%) and 242 (M+1, 33%). C₇H₇ClFNO₃S requires 239].

To an ice-cooled mixture of the crude mesylate (4.53 g, 18.90 mmol) in THF (180 ml), LAH (18.90 ml of a 1.0 M solution in THF, 18.90 mmol) was added dropwise and the reaction was stirred for 1 h. A 2 M HCl aqueous solution (80 ml) was added, the resulting mixture stirred for 30 min and then DCM (400 ml) was added. The organic layer was separated and evaporated to give the title compound D38 (2.28 g, 12.84 mmol, 67.9% yield from (2-chloro-5-fluoro-3-pyridinyl)methanol, two steps) as a white solid.

HPLC (walk-up): rt=3.56 min.

¹H NMR (400 MHz, DMSO-d₆) δ (ppm): 8.31 (d, 1H), 7.86 (dd, 1H), 2.35 (s, 3H).

### Description 39

### 5-fluoro-3-methyl-2-pyridinamine (D39)

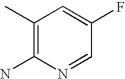

To a solution of 2-chloro-5-fluoro-3-methylpyridine D38 (0.50 g, 2.82 mmol) in dry toluene (12.5 ml) were added sodium t-butoxyde (0.462 g, 4.81 mmol), Pd₂(dba)₃ (0.315 g, 0.344 mmol), BINAP (0.642 g, 1.031 mmol) and benzophenone imine (0.692 ml, 4.12 mmol). The resulting mixture was degassed (3×pump/N₂) and then heated to 80° C. After 1 h stirring, the mixture was cooled down to room temperature, diluted with Et₂O (400 ml) and filtered through a celite pad. Volatiles were evaporated, the resulting oil was dissolved in THF (34 ml) and HCl (1.408 ml of a 2 M aqueous solution, 2.82 mmol) was added. The mixture was stirred at room temperature for 1.5 h, then neutralized with a saturated NaHCO₃ aqueous solution and diluted with DCM (200 ml). The inorganic layer was back-extracted with DCM (2×50 ml). The collected organic layers were dried (Na₂SO₄), filtered and evaporated. The residue was purified by flash chromatography on silica gel (Biotage SP4 12M, Cy/EtOAc 60/40). Collected fractions gave the title compound D39 (0.20 g, 1.554 mmol, 55.2% yield from D38, two steps), as an orange solid. MS: (ES/+) m/z: 127 (M+1). C₆H₇FN₂ requires 126.

¹H NMR (400 MHz, DMSO-d₆) δ(ppm): 7.73 (d, 1H), 7.23 (dd, 1H), 5.60 (bs, 2H), 2.04 (s, 3H).

### Description 40a

### 6-fluoro-8-methyl-2-[(2S)-2-piperidinylmethyl]imidazo[1,2-a]pyridine (free base) (D40a)

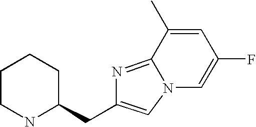

To a solution of 1,1-dimethylethyl (2S)-2-(3-bromo-2-oxopropyl)-1-piperidinecarboxylate D2 (0.15 g, 0.468 mmol) in DMF (1 ml) was added 5-fluoro-3-methyl-2-pyridinamine D39 (0.0709 g, 0.562 mmol) and the mixture was stirred at 80° C. for 1 h. The reaction mixture was eluted through a SCX column. Collected fractions gave 0.137 g of an oil containing a mixture of the title compound, the corresponding N-Boc protected derivative and some residual 5-fluoro-3-methyl-2-pyridinamine. [N-Boc derivative data. UPLC: rt=0.56 min, peak observed: 348 (M+1). C₁₉H₂₆FN₃O₂ requires 347]. The crude was dissolved in DCM (2 ml) and the resulting solution cooled to 0° C. TFA (0.40 ml) was added dropwise, the reaction left under stirring for 1 h and then eluted through a SCX column. Collected fractions gave the title compound as a free base D40a (0.093 g) contaminated with 5-fluoro-3-methyl-2-pyridinamine. The material was used without further purification in the next step. UPLC: rt=0.35 min, peak observed: 248 (M+1). C₁₄H₁₈FN₃ requires 247.

### Description 40b

### 6-fluoro-8-methyl-2-[(2S)-2-piperidinylmethyl]imidazo[1,2-a]pyridine (2HCl salt) (D40b)

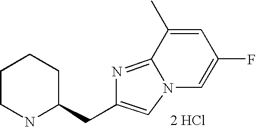

A mixture of 1,1-dimethylethyl (2S)-2-(3-bromo-2-oxopropyl)-1-piperidinecarboxylate D2 (0.94 g, 2.93 mmol; prepared by the method of D2 preparation (iii)), 5-fluoro-3-methyl-2-pyridinamine D39 (0.41 g, 3.25 mmol) and NaHCO₃ (0.37 g, 4.40 mmol) in toluene (4.70 ml) was stirred at 90° C. overnight. The mixture was allowed to cool down to room temperature and the inorganic salts were removed by filtration. The solid cake was washed with toluene (2×0.94 ml).

HCl (5-6 N solution in IPA, 2.22 ml, 11.10-13.32 mmol) was added to 5.18 g of the toluene solution (filtrate, 5.46 g) of the free base D40a. The mixture was heated to 70° C. and the resulting slurry stirred at that temperature under nitrogen atmosphere for 1 h. The slurry was aged at 70° C. for 1 h, cooled down to 40° C. over 2 h, allowed to reach room temperature and then stirred at that temperature overnight. The slurry was cooled down to 0° C. and aged at that temperature for 1 h. The solid was collected by filtration, washed with IPA (2×1.9 ml) and dried under vacuo at 40° C. for 4 h to afford the title compound D40b (0.53 g, 1.75 mmol, 59% yield). ¹H NMR (600 MHz, DMSO-d₆) δ (ppm): 15.18 (bs, 1H), 9.21 (bs, 1H), 9.07 (bs, 1H), 8.99 (s, 1H), 8.14 (s, 1H), 7.83 (bs, 1H), 3.15-3.65 (m, 4H), 2.61 (s, 3H), 1.85 (d, 1H), 1.69-1.79 (m, 2H), 1.48-1.67 (m, 2H), 1.38-1.48 (m, 1H). HPLC (walk-up, 3 min method): rt=1.28 min.

### Description 41

### 2-chloro-3-ethenyl-5-fluoropyridine (D41)

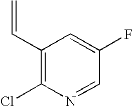

To a suspension of methyltriphenylphosphonium bromide (0.68 g, 1.92 mmol) in anhydrous THF (20 ml), n-BuLi (1.06 ml of a 1.6 M solution in Cy, 1.69 mmol) was added under nitrogen at −78° C. The cold bath was then removed and the reaction was allowed to reach room temperature and stirred for 1 h. To the resulting suspension at 0° C., a solution of 2-chloro-5-fluoro-3-pyridinecarbaldehyde (0.18 g, 1.13 mmol) dissolved in THF (10 ml) was slowly added. Stirring was maintained at room temperature for 4 h. The reaction was quenched with water (8 ml), the two phases were separated and the aqueous layer back-extracted with DCM. The organic phase was dried (Na₂SO₄) and the solvent was removed under reduced pressure. Purification by flash chromatography on silica gel (Cy/EtOAc 95/5) gave the title compound D41 (0.05 g, 0.27 mmol, 24% yield).

UPLC: rt=0.70 min, peaks observed: 158 (M+1, 100%) and 160 (M+1, 33%). C₇H₅ClFN requires 157. ¹H NMR (400 MHz, CDCl₃) δ (ppm): 8.20 (d, 1H), 7.62 (dd, 1H), 7.01 (ddd, 1H), 5.83 (d, 1H), 5.59 (d, 1H).

### Description 42

### 3-ethenyl-5-fluoro-2-pyridinamine (D42)

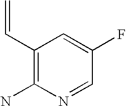

To a solution of 2-chloro-3-ethenyl-5-fluoropyridine D41 (0.045 g, 0.29 mmol) in toluene (2 ml), sodium t-butoxide (0.039 g, 0.40 mmol), Pd₂(dba)₃ (0.026 g, 0.03 mmol), BINAP (0.054 g, 0.09 mmol) and benzophenone imine (0.06 ml, 0.35 mmol) were added. The resulting mixture was degassed (3×pump/N₂) and then heated to 80° C. After 1 h stirring, the mixture was cooled to room temperature, diluted with Et₂O (50 ml) and filtered through a celite pad. After solvent evaporation the resulting oil was dissolved in THF (10 ml), a 2 M HCl aqueous solution (0.22 ml, 0.43 mmol) was added and the mixture stirred at room temperature for 2 h. Volatiles were evaporated. A saturated NaHCO₃ aqueous solution and DCM (50 ml) were added to the residue. The two layers were separated and the aqueous layer was back-extracted with DCM (2×50 ml). The collected organic layers were filtered through a phase separator tube and evaporated. The crude oil was purified by flash chromatography on silica gel (Biotage SP1 40M, Cy/EtOAc 60/40). Collected fractions gave the title compound D42 (0.013 g, 0.10 mmol, 34% yield from D41, two steps).

UPLC: rt=0.35 min, peak observed: 139 (M+1). C₇H₇FN₂ requires 138.

¹H NMR (400 MHz, CDCl₃) δ (ppm): 7.90 (d, 1H), 7.32 (dd, 1H), 6.62 (dd, 1H), 5.71 (dd, 1H), 5.48 (dd, 1H), 4.44 (bs, 2H).

### Description 43

### 8-ethenyl-6-fluoro-2-[(2S)-2-piperidinylmethyl]imidazo[1,2-a]pyridine (D43)

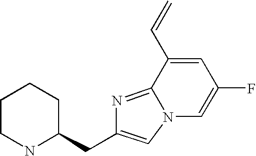

To a solution of 3-ethenyl-5-fluoro-2-pyridinamine D42 (0.013 g, 0.10 mmol) in DMF (1 ml), 1,1-dimethylethyl (2S)-2-(3-bromo-2-oxopropyl)-1-piperidinecarboxylate D2 (0.040 g, 0.13 mmol) was added and the reaction mixture left under stirring at 60° C. for 1 h and then at 80° C. for 4 h. The solvent was removed under vacuum and the crude eluted through a SCX column. The collected fractions gave a crude (0.022 g) containing the title compound and the corresponding N-Boc protected derivative. The material was used in the next step without further purification. [N-Boc derivative data. UPLC: rt=0.63 min, peak observed: 360 (M+1). C₂₀H₂₆FN₃O₂ requires 359]. The crude (0.022 g) was dissolved in DCM (1.50 ml) and TFA (0.38 ml) was added at 0° C. The reaction was left under stirring for 1 h, then volatiles were removed under vacuum and the residue eluted through a SCX column. Collected fractions gave the title compound D43 (0.016 g, 0.051 mmol, 51% yield from D42, two steps). UPLC: rt=0.42 min, peak observed: 260 (M+1). C₁₅H₁₈FN₃ requires 259.

### Description 44

### 3-ethyl-5-fluoro-2-pyridinamine (D44)

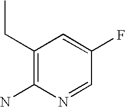

A mixture of 3-ethenyl-5-fluoro-2-pyridinamine D42 (0.23 g, 1.64 mmol) and PtO₂ (0.037 g, 0.16 mmol) in EtOH (15 ml) was stirred under hydrogen atmosphere (1 atm) for 15 min. The mixture was filtered through a celite pad and the solvent removed under vacuum to give the title compound D44 (0.21 g, 1.39 mmol, 84% yield) as a brown solid. UPLC: rt=0.34 min, peak observed: 141 (M+1). C₇H₉FN₂ requires 140.

¹H NMR (400 MHz, CDCl₃) δ (ppm): 7.82 (d, 1H), 7.12 (dd, 1H), 4.33 (bs, 2H), 2.46 (q, 2H), 1.28 (t, 3H).

### Description 45

### 8-ethyl-6-fluoro-2-[(2S)-2-piperidinylmethyl]imidazo[1,2-a]pyridine (D45)

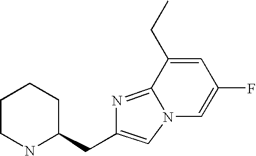

To a solution of 3-ethyl-5-fluoro-2-pyridinamine D44 (0.044 g, 0.31 mmol) in DMF (2 ml), 1,1-dimethylethyl (2S)-2-(3-bromo-2-oxopropyl)-1-piperidinecarboxylate D2 (0.10 g, 0.31 mmol) was added and the resulting mixture was left under stirring at 80° C. for 4 h. The solvent was removed under vacuum and the crude oil purified by flash chromatography on silica gel (DCM/MeOH from 100/0 to 98/2). Collected fractions gave a crude that was eluted through a SCX column to give, after solvent removal, a crude oil (0.071 g) containing the title compound and the corresponding N-Boc protected derivative. The material was used in the next step without further purification. [N-Boc derivative data. UPLC: rt=0.61 min, peak observed: 362 (M+1). C₂₀H₂₈FN₃O₂ requires 361]. The crude (0.071 g) was dissolved in DCM (1.50 ml) and TFA (0.38 ml) was added at 0° C. The reaction was left under stirring for 1 h, then volatiles were removed under vacuum and the residue eluted through a SCX column. Collected fractions gave the title compound D45 (0.050 g, 0.18 mmol, 58% yield from D2, two steps).

HPLC (walk-up): rt=2.41 min. UPLC: rt=0.36 min, peak observed: 262 (M+1). C₁₅H₂₀FN₃ requires 261.

### Description 46

### 6-chloro-5-(methyloxy)-3-pyridinamine (D46)

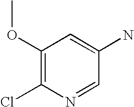

To a stirred solution of 2-chloro-3-(methyloxy)-5-nitropyridine (3.00 g, 15.90 mmol) in EtOAc (75 ml) was added SnCl₂ dihydrate (21.54 g, 95.00 mmol) and the resulting mixture was stirred at room temperature for 1 h. The reaction mixture was quenched with aqueous NaOH and extracted with EtOAc (5×75 ml). The collected organic layers were washed with water (3×75 ml), dried (Na₂SO₄), filtered and evaporated under reduced pressure to give the title compound D46 (2.34 g, 14.80 mmol, 93% yield) as a brown solid.

UPLC: rt=0.43 min, peaks observed: 159 (M+1, 100%) and 161 (M+1, 33%). C₆H₇ClN₂O requires 158.

¹H NMR (400 MHz, DMSO-d₆) δ (ppm): 7.29 (d, 1H), 6.71 (d, 1H), 5.50 (bs, 2H), 3.77 (s, 3H).

### Description 47

### 2-chloro-5-fluoro-3-(methyloxy)pyridine (D47)

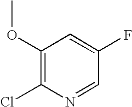

To an ice-cooled suspension of 6-chloro-5-(methyloxy)-3-pyridinamine D46 (2.14 g, 13.50 mmol) in HCl 4 M in water (10.12 ml, 40.50 mmol), a solution of sodium nitrite (1.02 g, 14.84 mmol) in water (7 ml) was added dropwise over a 5 min period and the resulting mixture was vigorously stirred at 5° C. for 30 min. To the mixture at 5° C. was added a solution of NaBF₄ (2.67 g, 24.29 mmol) in water (17 ml). The thick suspension was collected by filtration, washed with cold water and a little amount of cold EtOH and dried under reduced pressure at 55° C. for 8 h. The resulting black solid was taken-up in xylenes (25 ml) and allowed to reflux for 1 h. The solvent was evaporated under reduced pressure, the residue dissolved in EtOAc and washed with a saturated NaHCO₃ aqueous solution. The organic phase was separated, dried (Na₂SO₄), filtered and the solvent removed under vacuum. The resulting black oil was purified by flash chromatography on silica gel (Biotage SP4 25M, Cy/EtOAc 95/5) to afford the title compound D47 (0.11 g, 0.69 mmol, 5% yield) as a pale yellow solid. ¹H NMR (400 MHz, DMSO-d₆) δ (ppm): 8.03 (d, 1H), 7.70 (dd, 1H), 3.92 (s, 3H).

### Description 48

### 5-fluoro-3-(methyloxy)-2-pyridinamine (D48)

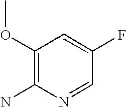

To a solution of 2-chloro-5-fluoro-3-(methyloxy)pyridine D47 (0.11 g, 0.70 mmol) in dry toluene (3 ml), sodium t-butoxide (0.094 g, 0.98 mmol), Pd₂(dba)₃ (0.064 g, 0.07 mmol), BINAP (0.131 g, 0.21 mmol) and benzophenone imine (0.14 ml, 0.84 mmol) were added. The resulting mixture was degassed (3×pump/N₂) and then heated to 80° C. After 1 h stirring, the mixture was cooled down to room temperature, diluted with Et₂O (80 ml) and filtered through a celite pad. Volatiles were evaporated, the resulting oil was dissolved in THF (8 ml) and HCl (0.35 ml of a 2 M aqueous solution, 0.70 mmol) was added. The mixture was stirred at room temperature for 1.5 h, then neutralized with a saturated NaHCO₃ aqueous solution and diluted with DCM (40 ml). The phases were separated and the aqueous one back-extracted with DCM (2×10 ml). The collected organic layers were dried (Na₂SO₄), filtered and evaporated. The residue was purified by flash chromatography on silica gel (Biotage SP4 12M, Cy/EtOAc 60/40) to give the title compound D48 (0.071 g, 0.49 mmol, 70% yield from D47, two steps) as a yellow solid. UPLC: rt=0.28 min, peak observed: 143 (M+1). C₆H₇FN₂O requires 142.

### Description 49

### 6-fluoro-8-(methyloxy)-2-[(2S)-2-piperidinylmethyl]imidazo[1,2-a]pyridine (D49)

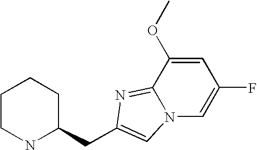

To a solution of 1,1-dimethylethyl (2S)-2-(3-bromo-2-oxopropyl)-1-piperidinecarboxylate D2 (0.19 g, 0.60 mmol) in DMF (1 ml), 5-fluoro-3-(methyloxy)-2-pyridinamine D48 (0.071 g, 0.50 mmol) was added and the mixture stirred at 80° C. for 2 h. The reaction mixture was eluted through a SCX column. Collected fractions gave 0.14 g of a crude oil containing a mixture of the title compound, the corresponding N-Boc protected derivative and some residual 5-fluoro-3-(methyloxy)-2-pyridinamine. The material was used in the next step without further purification. [N-Boc derivative data. MS: (ES/+) m/z: 364 (M+1). C₁₉H₂₆FN₃O₃ requires 363]. The crude (0.14 g) was dissolved in DCM (2 ml) and TFA (0.40 ml) was added at 0° C. The reaction was left under stirring for 1 h, then volatiles were removed under vacuum and the residue eluted through a SCX column. Collected fractions gave an oil (0.13 g) containing the title compound D49. The material was used in the next step without further purification. UPLC: rt=0.33 min, peak observed: 264 (M+1). C₁₄H₁₈FN₃O requires 263.

### Description 50

### 3-({[(1,1-dimethylethyl)(dimethyl)silyl]oxy}methyl)-5-fluoro-2-pyridinamine (D50)

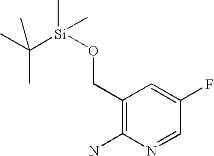

2-chloro-(5-fluoro-3-pyridinyl)methanol (0.40 g, 2.45 mmol) was dissolved in DMF (10 ml), then imidazole (0.50 g, 7.36 mmol) and TBSCl (0.41 g, 2.70 mmol) were added and the reaction left under stirring at room temperature. After 2 h an additional equivalent of TBSCl was added and the solution stirred overnight. The mixture was diluted with Et₂O and washed with water and brine. The organic phase was dried (Na₂SO₄) and concentrated to give the O-TBS protected chloro pyridine as a crude (0.73 g). The material was used in the next step without further purification. [O-TBS derivative data. ¹H NMR (400 MHz, CDCl₃) δ (ppm): 8.17 (dt, 1H), 7.66-7.71 (m, 1H), 4.73 (s, 2H), 1.00 (s, 9H), 0.18 (s, 6H)].

To a solution of the crude material (0.73 g) in dry toluene (10 ml), sodium t-butoxide (0.36 g, 3.73 mmol), Pd₂(dba)₃ (0.24 g, 0.27 mmol), BINAP (0.50 g, 0.80 mmol) and benzophenone imine (0.54 ml, 3.19 mmol) were added. The resulting mixture was degassed (3×pump/N₂) and then heated at 80° C. for 1 h. The mixture was cooled down to room temperature, diluted with Et₂O (100 ml), filtered through a celite pad and the solvents removed under reduced pressure to give a crude oil. The material was dissolved in THF (80 ml), a 2 M HCl aqueous solution (2.66 ml, 5.32 mmol) was added and the mixture stirred at room temperature for 30 min. Volatiles were evaporated. A saturated NaHCO₃ aqueous solution and DCM (300 ml) were added. The two layers were separated and the aqueous one back-extracted with DCM (3×200 ml). The combined organic phases were filtered through a phase separator tube and evaporated. The red oil obtained was purified by flash chromatography on silica gel (Biotage SP1 40M, Cy/EtOAc 90/10). Collected fractions gave the title compound D50 (0.29 g, 1.14 mmol, 46% yield from 2-chloro-(5-fluoro-3-pyridinyl)methanol, three steps). ¹H NMR (400 MHz, CDCl₃) δ (ppm): 7.89 (d, 1H), 7.15 (dd, 1H), 4.76 (bs, 2H), 4.59 (s, 2H), 0.93 (s, 9H), 0.12 (s, 6H).

### Description 51

### {6-fluoro-2-[(2S)-2-piperidinylmethyl]imidazo[1,2-a]pyridin-8-yl}methanol (D51)

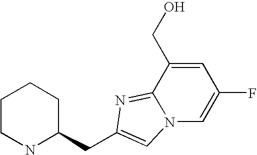

To a solution of 1,1-dimethylethyl (2S)-2-(3-bromo-2-oxopropyl)-1-piperidinecarboxylate D2 (0.10 g, 0.31 mmol) in DMF (2.50 ml), 3-({[(1,1-dimethylethyl)(dimethyl)silyl]oxy}methyl)-5-fluoro-2-pyridinamine D50 (0.088 g, 0.34 mmol) was added and the reaction left under stirring at 70° C. for 2 h. The solvent was removed under vacuum and the residue eluted through a SCX column. Collected fractions gave a crude (0.067 g) containing a mixture of the title compound and the corresponding N-Boc protected derivative. The material was used in the next step without further purification. [N-Boc derivative data. UPLC: rt=0.56 min, peak observed: 364 (M+1). C₁₉H₂₆FN₃O₃ requires 363]. The crude (0.067 g) was dissolved in DCM (5 ml) and TFA (1 ml) was added dropwise at 0° C. The reaction was left under stirring at room temperature for 1 h, then volatiles were removed under vacuum and the residue eluted through a SCX column. Collected fractions gave the title compound D51 (0.060 g, 0.19 mmol, 61% yield from D2, two steps) contaminated with some residual (2-amino-5-fluoro-3-pyridinyl)methanol.

UPLC: rt=0.31 min, peak observed: 264 (M+1). C₁₄H₁₈FN₃O requires 263.

### Description 52

### 5-fluoro-3-[(methyloxy)methyl]-2-pyridinamine (D52)

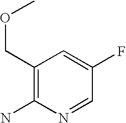

To a solution of (2-chloro-5-fluoro-3-pyridinyl)methanol (1.10 g, 6.81 mmol) in THF (15 ml), NaH (0.41 g of a 60% wt mineral oil dispersion, 10.21 mmol) was added portionwise at 0° C. and the resulting mixture was left under stirring at room temperature for 45 min. The mixture was cooled down to 0° C. and methyl iodide (0.47 ml, 7.49 mmol) was added dropwise. After 4 h stirring at room temperature the mixture was diluted with EtOAc and washed with a 0.5 M NaOH aqueous solution. The two phases were separated and the organic one dried (Na₂SO₄), filtered and the solvent removed under vacuum to give the intermediate 2-chloro-5-fluoro-3-[(methyloxy)methyl]pyridine as a crude yellow oil (1.24 g) that was used in the next step without further purification. [Chloropyridine data.

UPLC: rt=0.65 min, peaks observed: 176 (M+1, 100%) and 178 (M+1, 33%). C₇H₇ClFNO requires 175]. ¹H NMR (400 MHz, DMSO-d₆) δ (ppm): 8.42 (d, 1H), 7.82 (dd, 1H), 4.49 (s, 2H), 3.42 (s, 3H)]. The crude material (1.24 g) was dissolved in dry toluene (17 ml) and sodium t-butoxide (0.95 g, 9.89 mmol), Pd₂(dba)₃ (0.65 g, 0.71 mmol), BINAP (1.32 g, 2.12 mmol) and benzophenone imine (1.42 ml, 8.47 mmol) were added. The resulting mixture was degassed (3×pump/N₂) and then heated to 80° C. for 1 h. The mixture was cooled to room temperature, diluted with Et₂O (800 ml), filtered through a celite pad and the solvents removed under reduced pressure. The crude oil was dissolved in THF (70 ml), a 2 M HCl aqueous solution (3.53 ml, 7.06 mmol) was added and the mixture stirred at room temperature overnight. Volatiles were evaporated. A saturated NaHCO₃ aqueous solution and DCM (300 ml) were added. The two layers were separated and the aqueous one was back-extracted with DCM (2×200 ml). The combined organic phases were filtered through a phase separator tube and evaporated to give a red oil that was purified by flash chromatography on silica gel (Biotage SP1 40M, Cy/EtOAc 60/40). Collected fractions gave the title compound D52 (0.72 g, 4.58 mmol, 67% yield from (2-chloro-5-fluoro-3-pyridinyl)methanol, three steps). HPLC (walk-up): rt=0.92 min. UPLC: rt=0.33 min, peak observed: 157 (M+1). C₇H₉FN₂O requires 156. ¹H NMR (400 MHz, DMSO-d₆) δ (ppm): 7.85 (d, 1H), 7.33 (dd, 1H), 5.66 (bs, 2H), 4.27 (s, 2H), 3.31 (s, 3H).

### Description 53

### 6-fluoro-8-[(methyloxy)methyl]-2-[(2S)-2-piperidinylmethyl]imidazo[1,2-a]pyridine (D53)

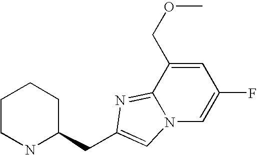

To a solution of 1,1-dimethylethyl (2S)-2-(3-bromo-2-oxopropyl)-1-piperidinecarboxylate D2 (0.13 g, 0.42 mmol) in DMF (1.50 ml), 5-fluoro-3-[(methyloxy)methyl]-2-pyridinamine D52 (0.078 g, 0.50 mmol) was added. The reaction was left under stirring at 60° C. for 1.5 h and at 80° C. for an additional 1.5 h. DCM was added and the mixture washed with brine and water. The two phases were separated and the organic one was filtered through a phase separator tube. The solvent was removed under vacuum and the residue eluted through a SCX column to give a crude (0.13 g) containing a mixture of the title compound, the corresponding N-Boc protected derivative and some residual 5-fluoro-3-[(methyloxy)methyl]-2-pyridinamine. The material was used in the next step without further purification. [N-Boc derivative data. UPLC: rt=0.58 min, peak observed: 378 (M+1). C₂₀H₂₈FN₃O₃ requires 377]. The crude (0.13 g) was dissolved in DCM (8 ml) and TFA (2 ml) was added dropwise at 0° C. The reaction was left under stirring at room temperature for 2 h, the solvent was removed under vacuum and the residue eluted through a SCX column. Collected fractions gave the title compound D53 contaminated with some residual 5-fluoro-3-[(methyloxy)methyl]-2-pyridinamine (0.10 g, 0.34 mmol, 81% yield from D2, two steps). HPLC (walk-up): rt=1.92 min. UPLC: rt=0.37 min, peak observed: 278 (M+1). C₁₅H₂₀FN₃O requires 277.

### Description 54

### 3-chloro-2-pyridinamine (D54)

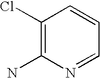

To a stirred solution of 3-chloro-2-nitropyridine (1.00 g, 6.31 mmol) in EtOH (13 ml) were added a 2 M HCl aqueous solution (1.30 ml, 2.60 mmol) and iron (2.37 g, 42.4 mmol) at 0° C. The resulting mixture was stirred for 2.5 h at room temperature. Celite (2.40 g) was added. The mixture was filtered over a celite pad and evaporated to give a dark oil that was purified by elution through a SCX cartridge. The title compound D54 (0.34 g, 2.59 mmol, 41% yield) was obtained as a dark solid. UPLC: rt=0.27 min, peaks observed: 129 (M+1, 100%) and 131 (M+1, 33%). C₅H₅ClN₂ requires 128.

### Description 55

### 8-chloro-2-[(2S)-2-piperidinylmethyl]imidazo[1,2-a]pyridine (D55)

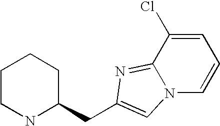

To a solution of 1,1-dimethylethyl (2S)-2-(3-bromo-2-oxopropyl)-1-piperidinecarboxylate D2 (0.15 g, 0.47 mmol) in DMF (1 ml) was added 3-chloro-2-pyridinamine D54 (0.072 g, 0.56 mmol) and the mixture was stirred at 80° C. for 1 h. The reaction mixture was purified via elution through a SCX cartridge. Collected fractions gave a crude (0.13 g) containing a mixture of the title compound, the corresponding N-Boc protected derivative and some residual 3-chloro-2-pyridinamine. The material was used in the next step without further purification. [N-Boc derivative data. UPLC: rt=0.57 min, peaks observed: 350 (M+1, 100%) and 352 (M+1, 33%). C₁₈H₂₄ClN₃O₂ requires 349]. The crude material (0.13 g) was dissolved in DCM (2 ml) and TFA (0.40 ml) was added dropwise at 0° C. The solution was left under stirring for 1 h, then volatiles were removed under reduced pressure and the residue purified by elution through a SCX cartridge. Collected fractions gave the title compound D55 (0.088 g) as a brown oil contaminated with 3-chloro-2-pyridinamine. The material was used in the next step without further purification. UPLC: rt=0.37 min, peaks observed: 250 (M+1, 100%) and 252 (M+1, 33%). C₁₃H₁₆ClN₃ requires 249.

### Description 56

### 3-[(2,2,2-trifluoroethyl)oxy]-2-pyridinamine (D56)

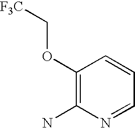

To a stirred solution of 2-amino-3-pyridinol (1.00 g, 9.08 mmol) in DMF (8 ml), NaH (0.40 g of a 60% wt mineral oil dispersion, 9.99 mmol) and 1,1,1-trifluoro-2-iodoethane (2.69 ml, 27.2 mmol) were added. The resulting mixture was stirred at 55° C. overnight. The solvent was evaporated under reduced pressure and the resulting black oil was taken-up in DCM (300 ml) and washed with water/brine (1 l). The aqueous phase was back-extracted with DCM (3×300 ml). The collected organic phases were concentrated under vacuum, washed with brine (2×15 ml), separated in a phase separator tube and evaporated to give the title compound D56 (1.40 g, 5.83 mmol, 64% yield) as a brown solid. UPLC: rt=0.35 min, peak observed: 193 (M+1). C₇H₇F₃N₂O requires 192.

### Description 57

### 2-[(2S)-2-piperidinylmethyl]-8-[(2,2,2-trifluoroethyl)oxy]imidazo[1,2-a]pyridine (D57)

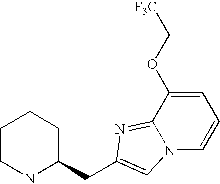

To a solution of 1,1-dimethylethyl (2S)-2-(3-bromo-2-oxopropyl)-1-piperidinecarboxylate D2 (0.15 g, 0.47 mmol) in DMF (1 ml) was added 3-[(2,2,2-trifluoroethyl)oxy]-2-pyridinamine D56 (0.11 g, 0.56 mmol) and the mixture was stirred at 80° C. for 1 h. The reaction mixture was purified via elution through a SCX cartridge. Collected fractions gave a crude (0.13 g) containing a mixture of the title compound, the corresponding N-Boc protected derivative and some residual 3-[(2,2,2-trifluoroethyl)oxy]-2-pyridinamine. The material was used in the next step without further purification. [N-Boc derivative data. UPLC: rt=0.62 min, peak observed: 414 (M+1). C₂₀H₂₆F₃N₃O₃ requires 413]. The crude material (0.13 g) was dissolved in DCM (2 ml) and TFA (0.40 ml) was added dropwise at 0° C. The mixture was stirred for 1 h, volatiles were removed under reduced pressure and the residue purified by elution through a SCX cartridge. Collected fractions gave the title compound D57 (0.096 g, 0.31 mmol, 65% yield from D2, two steps) contaminated with some residual 3-[(2,2,2-trifluoroethyl)oxy]-2-pyridinamine as a brown oil. UPLC: rt=0.38 min, peak observed: 314 (M+1). C₁₅H₁₈F₃N₃O requires 313.

### Description 58

### 8-fluoro-2-[(2S)-2-piperidinylmethyl]imidazo[1,2-a]pyridine (HCl salt) (D58)

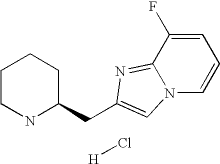

1,1-dimethylethyl (2S)-2-[(8-fluoroimidazo[1,2-a]pyridin-2-yl)methyl]-1-piperidinecarboxylate D19 (23.56 g, 70.70 mmol) was dissolved in DCM (35 ml) and the resulting solution cooled to 10° C. under Argon atmosphere. A 4 M HCl solution in 1,4-dioxane (148 ml, 594 mmol) was added dropwise, the reaction allowed to warm-up to room temperature and left under stirring for 2.15 h. Volatiles were removed under vacuo and the residue triturated with Et₂O (2×250 ml) to give the title compound D58 (23.796 g) as a white solid. The material contained some residual 1,4-dioxane and 3-fluoro-2-pyridinamine (the overall recovered amount was higher than the theoretical amount) and was used in the next step without further purification. UPLC: rt 0.33 min, peak observed: 234 (M+1-HCl). C₁₃H₁₇FClN₃ requires 269.

### Description 59

### 1,1-dimethylethyl (2S)-2-[(8-fluoro-3-iodoimidazo[1,2-a]pyridin-2-yl)methyl]-1-piperidinecarboxylate (D59)

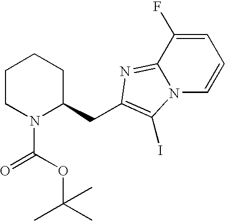

To a solution of 1,1-dimethylethyl (2S)-2-[(8-fluoroimidazo[1,2-a]pyridin-2-yl)methyl]-1-piperidinecarboxylate D19 (0.25 g, 0.75 mmol) in DCM (80 ml), 12 (23.60 ml of a 1 M DCM solution, 23.60 mmol) was added dropwise at room temperature and the resulting mixture was stirred for 3 h. A 5% NaHSO₃ aqueous solution (20 ml) was added and the mixture vigorously stirred for 10 min. The organic phase was separated, dried (Na₂SO₄), filtered and concentrated to give a yellow solid that was purified on NH by flash chromatography (Biotage SP4 25M, from Cy 100 to Cy/EtOAc 70/30). Collected fractions gave the title compound D59 (0.28 g, 0.60 mmol, 80% yield). UPLC: rt=0.78 min, peak observed: 460 (M+1). C₁₈H₂₃FIN₃O₂ requires 459.

### Description 60

### 1,1-dimethylethyl (2S)-2-[(8-fluoro-3-methylimidazo[1,2-a]pyridin-2-yl)methyl]-1-piperidinecarboxylate (D60)

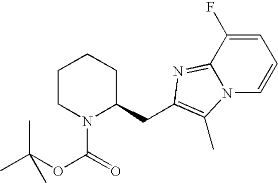

To a mixture of 1,1-dimethylethyl (2S)-2-[(8-fluoro-3-iodoimidazo[1,2-a]pyridin-2-yl)methyl]-1-piperidinecarboxylate D59 (0.28 g, 0.60 mmol) and palladium-tetrakis(triphenylphosphine) (0.035 g, 0.03 mmol) in DME (7.40 ml) was added methylboronic acid (0.054 g, 0.90 mmol) followed by the addition of NaOH (2.40 ml of a 0.5 M aqueous solution, 1.20 mmol). The resulting mixture was stirred at 110° C. for 40 min under microwave irradiation. The reaction mixture was poured into water (5 ml) and extracted with DCM (3×3 ml). The organic phases were collected, dried (Na₂SO₄), filtered and the solvent evaporated under vacuum. The green residue was purified on NH by flash chromatography (Biotage 25M, from Cy 100 to Cy/EtOAc 70/30). Collected fractions gave the title compound D60 (0.17 g, 0.47 mmol, 79% yield). MS: (ES/+) m/z: 348 (M+1). C₁₉H₂₆FN₃O₂ requires 347. HPLC (walk-up): rt=4.56 min.

### Description 61

### 8-fluoro-3-methyl-2-[(2S)-2-piperidinylmethyl]imidazo[1,2-a]pyridine (D61)

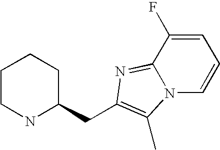

To a solution of 1,1-dimethylethyl (2S)-2-[(8-fluoro-3-methylimidazo[1,2-a]pyridin-2-yl)methyl]-1-piperidinecarboxylate D60 (0.17 g, 0.47 mmol) in DCM (4 ml), TFA (1 ml) was added and the solution stirred for 1.5 h. Volatiles were removed under reduced pressure and the residue eluted through a SCX column. Collected fractions gave the title compound D61 (0.11 g, 0.43 mmol, 91% yield). HPLC (walk-up): rt=2.66 min.

MS: (ES/+) m/z: 248 (M+1). C₁₄H₁₈FN₃ requires 247.

### Description 62

### 1,1-dimethylethyl (2S)-2-[(3-chloro-8-methylimidazo[1,2-a]pyridin-2-yl)methyl]-1-piperidinecarboxylate (D62)

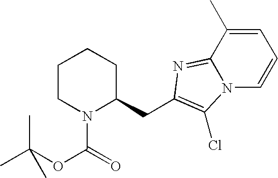

To a solution of 1,1-dimethylethyl (2S)-2-[(8-methylimidazo[1,2-a]pyridin-2-yl)methyl]-1-piperidinecarboxylate D11 (0.18 g, 0.56 mmol) in DCM (4 ml) was added NCS (0.082 g, 0.62 mmol) and the reaction mixture was stirred at room temperature for 30 min. The solvent was evaporated to afford the title compound D62 (0.29 g) as a crude material which was used in the next step without any further purification. UPLC: rt=0.68 min, peak observed: 364 (M+1). C₁₉H₂₆ClN₃O₂ requires 363.

### Description 63

### 3-chloro-8-methyl-2-[(2S)-2-piperidinylmethyl]imidazo[1,2-a]pyridine (D63)

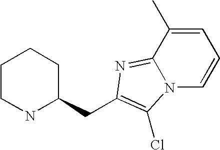

To a solution of 1, 1-dimethylethyl (2S)-2-[(3-chloro-8-methylimidazo[1,2-a]pyridin-2-yl)methyl]-1-piperidinecarboxylate D62 (0.29 g) in DCM (6 ml), TFA (1.20 ml) was added dropwise at 0° C. and the reaction mixture was stirred for 1 h. The solvent was evaporated and the residue eluted through a SCX column. Collected fractions gave the title compound D63 (0.17 g) as a crude material which was used in the next step without any further purification. UPLC: rt=0.43 min, peak observed: 264 (M+1). C₁₄H₁₈ClN₃ requires 263. HPLC (walk-up): rt=2.20 min.

### EXAMPLES

### Example 1

### 2-[((2S)-1-{[5-(4-fluorophenyl)-2-methyl-1,3-thiazol-4-yl]carbonyl}-2-piperidinyl)methyl]-7-(trifluoromethyl)imidazo[1,2-a]pyridine (E1)

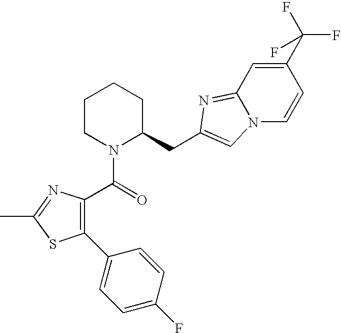

A mixture of 5-(4-fluorophenyl)-2-methyl-1,3-thiazole-4-carboxylic acid (0.23 g, 1.00 mmol), DIPEA (1.00 ml, 5.70 mmol) and TBTU (0.40 g, 1.24 mmol) in DMF (3 ml) was left under stirring at room temperature for 20 min. A 0.05 M solution of 2-[(2S)-2-piperidinylmethyl]-7-(trifluoromethyl)imidazo[1,2-a]pyridine D4 in DMF (2.40 ml, 0.12 mmol) was added to the activated carboxylic acid and the mixture was stirred for 1 h. Water was added and the mixture extracted with EtOAc. The resulting crude oil was submitted to Fraction Lynx purification (LC 3_{—}100 mg method). After two runs the title compound E1 (0.020 g, 0.04 mmol, 33% yield) was obtained. HPLC (walk-up): rt=4.07 min. MS: (ES/+) m/z: 503 (M+1). UPLC: rt=0.67 min, peak observed: 503 (M+1). C₂₅H₂₂F₄N₄OS requires 502.

### Example 2

### 2-({(2S)-1-[(2-methyl-5-phenyl-1,3-thiazol-4-yl)carbonyl]-2-piperidinyl}methyl)-7-(trifluoromethyl)imidazo[1,2-a]pyridine (E2)

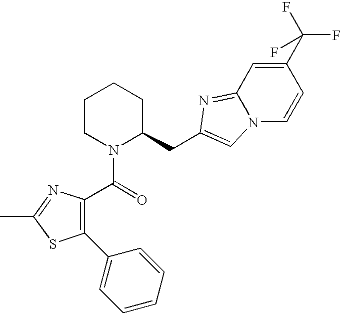

A mixture of 5-phenyl-2-methyl-1,3-thiazole-4-carboxylic acid (0.23 g, 1.00 mmol), DIPEA (1.00 ml, 5.70 mmol) and TBTU (0.40 g, 1.24 mmol) in DMF (3 ml) was left under stirring at room temperature for 20 min. A 0.05 M solution of 2-[(2S)-2-piperidinylmethyl]-7-(trifluoromethyl)imidazo[1,2-a]pyridine D4 (2.40 ml, 0.12 mmol) was added to the activated carboxylic acid and the reaction was stirred for 1 h. Water was added and the mixture extracted with EtOAc. The resulting crude oil was submitted to Fraction Lynx purification (LC 3_{—}100 mg method). After two runs the title compound E2 (0.038 g, 0.08 mmol, 66% yield) was obtained as a yellowish solid. HPLC (walk-up): rt=3.97 min. UPLC: rt=0.66 min, peak observed: 485 (M+1). C₂₅H₂₃F₃N₄OS requires 484.

### Example 3

### 2-({(2S)-1-[(2-methyl-5-phenyl-1,3-thiazol-4-yl)carbonyl]-2-piperidinyl}methyl)-6-(trifluoromethyl)imidazo[1,2-a]pyridine (E3)

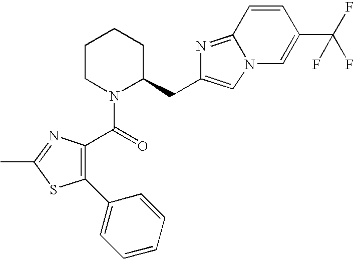

In a 5 ml round-bottomed flask 5-phenyl-2-methyl-1,3-thiazole-4-carboxylic acid (0.065 g, 0.30 mmol), DMF (1 ml), DIPEA (0.25 ml, 1.48 mmol) and TBTU (0.11 g, 0.36 mmol) were added and the mixture left under stirring at room temperature for 20 min. A solution of 2-[(2S)-2-piperidinylmethyl]-6-(trifluoromethyl)imidazo[1,2-a]pyridine D6 (0.070 g of the crude material obtained in Description 6) in DMF (1 ml) was added to the activated carboxylic acid and the reaction stirred for 1 h. Water was added and the mixture extracted with EtOAc. The organic phase was dried (Na₂SO₄) and the solvent removed under reduced pressure to give an oil that was eluted through a SCX column and then purified by chromatography on silica gel (Flash Master 50 g, DCM/MeOH from 100/0 to 80/20). Collected fractions gave the title compound E3 (0.009 g, 0.019 mmol, 12% from D2, three steps). MS: (ES/+) m/z: 485 (M+1). C₂₅H₂₃F₃N₄OS requires 484. HPLC (walk-up): rt=3.99 min.

### Example 4

### 2-({(2S)-1-[(2-methyl-5-phenyl-1,3-thiazol-4-yl)carbonyl]-2-piperidinyl}methyl)-8-(trifluoromethyl)imidazo[1,2-a]pyridine (E4)

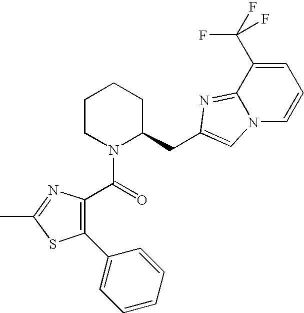

To a solution of 1,1-dimethylethyl (2S)-2-{[8-(trifluoromethyl)imidazo[1,2-a]pyridin-2-yl]methyl}-1-piperidinecarboxylate D7 (0.15 g contaminated with residual 3-(trifluoromethyl)-2-pyridinamine as reported in Description 7) in DCM (4 ml), TFA (2 ml) was added dropwise at 0° C. and the resulting reaction mixture was stirred at room temperature for 2 h. Solvent removal afforded a residue that was eluted through a SCX column. Collected fractions gave a crude (containing the intermediate N-Boc deprotected amine contaminated with some residual 3-(trifluoromethyl)-2-pyridinamine) that was dissolved in DMF (2 ml).

A mixture of 5-phenyl-2-methyl-1,3-thiazole-4-carboxylic acid (0.12 g, 0.55 mmol), DMF (2 ml), DIPEA (0.50 ml, 2.96 mmol) and TBTU (0.24 g, 0.75 mmol) was left under stirring at room temperature. A solution of the free amine in DMF was added dropwise and the reaction left under stirring at room temperature. Water was added and the mixture extracted with EtOAc. The resulting crude was purified by Fraction Lynx (LC 3_{—}100 mg method). The resulting material was then eluted through a SCX column. Collected fractions gave the title compound E4 (0.060 g, 0.12 mmol, 40% yield from D2, three steps).

MS: (ES/+) m/z: 485 (M+1). C₂₅H₂₃F₃N₄OS requires 484. HPLC (walk-up): rt=3.89 min.

### Example 5

### 6,8-dichloro-2-({(2S)-1-[(2-methyl-5-phenyl-1,3-thiazol-4-yl)carbonyl]-2-piperidinyl}methyl)imidazo[1,2-a]pyridine (E5)

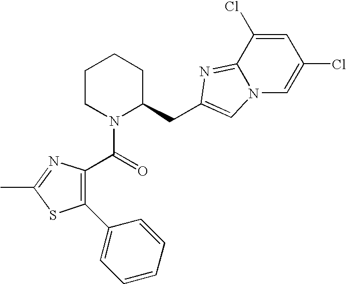

A mixture of 5-phenyl-2-methyl-1,3-thiazole-4-carboxylic acid (0.048 g, 0.22 mmol), DMF (0.50 ml), DIPEA (0.19 ml, 1.10 mmol) and TBTU (0.085 g, 0.26 mmol) was left under stirring at room temperature for 20 min. A solution of 6,8-dichloro-2-[(2S)-2-piperidinylmethyl]imidazo[1,2-a]pyridine D10 (0.051 g of the crude material obtained in Description 10) in DMF (1 ml) was added at 0° C. to the activated carboxylic acid and the reaction stirred for 1 h. The mixture was transferred into a separatory funnel containing brine (3 ml) and extracted with EtOAc (2×4 ml). The collected organic phases were washed with brine/ice (6×3 ml), dried (Na₂SO₄) and the solvent removed under reduced pressure to give an oil that was purified by MDAP Fraction Lynx. Collected fractions gave the title compound E5 (0.008 g, 0.016 mmol, 10% from D2, three steps). MS: (ES/+) m/z: 485 (M+1, 100%) and 487 (M+1, 66%). UPLC rt=3.00 min, peak observed: 485 (M+1) and 487 (M+1). C₂₄H₂₂Cl₂N₄OS requires 484.

### Example 6

### 8-methyl-2-({(2S)-1-[(2-methyl-5-phenyl-1,3-thiazol-4-yl)carbonyl]-2-piperidinyl}methyl)imidazo[1,2-a]pyridine (HCl salt) (E6)

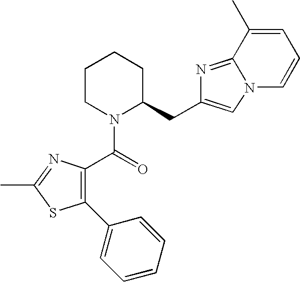

In a 100 ml pear flask 2-methyl-5-phenyl-1,3-thiazole-4-carboxylic acid (0.76 g, 3.49 mmol) was dissolved in DCM (15 ml) to give a yellow solution. DMF (0.014 ml, 0.17 mmol) was then added and the mixture cooled to 0° C. Oxalyl chloride (0.67 ml, 7.67 mmol) was added dropwise and the resulting mixture left under stirring at room temperature for 1 h. Volatiles were removed under reduced pressure and the residue dissolved in DCM (15 ml). The acyl chloride solution was added dropwise to a solution of 8-methyl-2-[(2S)-2-piperidinylmethyl]imidazo[1,2-a]pyridine D12 (0.80 g, 3.49 mmol) and TEA (1.46 ml, 10.47 mmol) in DCM (15 ml) cooled at 0° C. The reaction mixture was left under stirring overnight. DCM (30 ml) was added and the mixture washed with a saturated NaHCO₃ aqueous solution (70 ml). The two layers were separated and the aqueous one back-extracted with DCM (3×50 ml). The combined organic phases were washed with water (2×50 ml), dried (Na₂SO₄), filtered and concentrated. The residue was purified by chromatography on silica gel (Flash Master, DCM/MeOH/NH₃ from 90/10/0 to 90/10/0.2). The free base of the title compound (1.00 g, 2.32 mmol, 67% yield) was obtained as a slightly brown oil. HPLC (walk-up): rt=3.60 min.

The free base (1.00 g, 2.32 mmol) was dissolved in DCM (35 ml) and the solution cooled to 0° C. HCl (3.48 ml of a 1 M solution in Et₂O, 3.48 mmol) was added dropwise and the mixture allowed to warm up to room temperature and stirred for 1 h. Volatiles were removed under reduced pressure and the resulting solid triturated with Et₂O. The title compound E6 (1.05 g, 2.00 mmol, 86% yield) was obtained as a slightly yellow solid. UPLC: rt=0.59 min, peak observed: 431 (M+1). C₂₅H₂₆N₄OS requires 430.

### Example 7

### 6,8-difluoro-2-({(2S)-1-[(2-methyl-5-phenyl-1,3-thiazol-4-yl)carbonyl]-2-piperidinyl}methyl)imidazo[1,2-a]pyridine (E7)

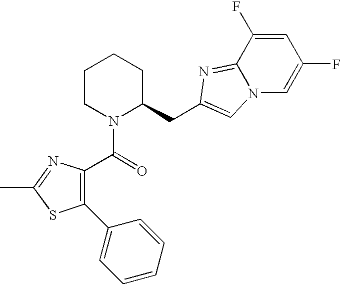

A mixture of 2-methyl-5-phenyl-1,3-thiazole-4-carboxylic acid (0.057 g, 0.26 mmol), DMF (3 ml), DIPEA (0.23 ml, 1.29 mmol) and TBTU (0.10 g, 0.31 mmol) was stirred at room temperature for 20 min. A solution of 6,8-difluoro-2-[(2S)-2-piperidinylmethyl]imidazo[1,2-a]pyridine D13 (0.054 g, 0.22 mmol) in DMF (1 ml) was added and the resulting mixture stirred overnight. The reaction mixture was diluted with brine (3 ml) and extracted with EtOAc (2×4 ml). The combined organic layers were washed with brine/ice (6×3 ml), dried (Na₂SO₄) and the solvent removed under vacuum. The crude was purified by flash chromatography on silica gel (Biotage SP 112 M, DCM/MeOH 95/5). Collected fractions gave the title compound E7 (0.034 g, 0.08 mmol, 35% yield) as a yellow solid.

MS: (ES/+) m/z: 453 (M+1). C₂₄H₂₂F₂N₄OS requires 452. ¹H NMR [the product is present as a mixture of conformers (ratio ca. 50/50) and the assignment refers to a single conformer] (500 MHz, CDCl₃) δ (ppm): 7.83-7.89 (m, 1H), 7.75-7.78 (m, 1H), 7.26-7.34 (m, 3H), 7.21 (t, 2H), 6.80-6.90 (m, 1H), 5.28-5.35 (m, 1H), 4.69-4.77 (m, 1H), 3.29 (dd, 1H), 3.08 (dd, 1H), 3.01 (dt, 1H), 2.70 (s, 3H), 1.29-1.73 (m, 5H), 0.92-1.04 (m, 1H).

### Example 8

### 6-fluoro-2-({(2S)-1-[(2-methyl-5-phenyl-1,3-thiazol-4-yl)carbonyl]-2-piperidinyl}methyl)imidazo[1,2-a]pyridine (E8)

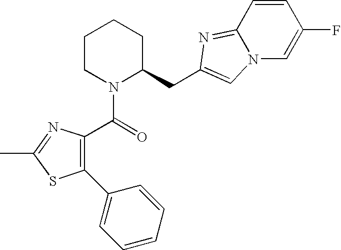

A mixture of 2-methyl-5-phenyl-1,3-thiazole-4-carboxylic acid (0.0575 g, 0.262 mmol), DMF (3 ml), DIPEA (0.229 ml, 1.314 mmol) and TBTU (0.101 g, 0.315 mmol) was stirred at room temperature for 20 min. A solution of 6-fluoro-2-[(2S)-2-piperidinylmethyl]imidazo[1,2-a]pyridine D14 (0.051 g, 0.219 mmol) in DMF (1 ml) was added and the mixture left under stirring overnight. The reaction mixture was diluted with brine (2.5 ml) and extracted with EtOAc (2×3.5 ml). The combined organic layers were washed with brine/ice (6×3 ml), dried (Na₂SO₄) and the solvent removed. The crude was purified by flash chromatography on silica gel (Biotage SP1 12M, DCM/MeOH 95/5). Collected fractions gave the title compound E8 (0.036 g, 0.083 mmol, 37.9% yield) as a yellow solid. MS: (ES/+) m/z: 435 (M+1). C₂₄H₂₃FN₄OS requires 434. ¹H NMR [the product is present as a mixture of conformers (ratio ca. 50/50) and the assignment refers to a single conformer] (500 MHz, CDCl₃) δ (ppm): 7.94-7.98 (m, 1H), 7.66 (s, 1H), 7.46-7.53 (m, 1H), 7.18-7.41 (m, 5H), 7.00-7.10 (m, 1H), 5.26-5.34 (m, 1H), 4.69-4.78 (m, 1H), 3.21 (dd, 1H), 3.06 (dd, 1 H), 2.90-2.99 (m, 1H), 2.72 (s, 3H), 1.26-1.76 (m, 5H), 0.92-1.05 (m, 1H).

### Example 9

### 2-({(2S)-1-[(2-methyl-5-phenyl-1,3-thiazol-4-yl)carbonyl]-2-piperidinyl}methyl)imidazo[1,2-a]pyridine-7-carbonitrile (E9)

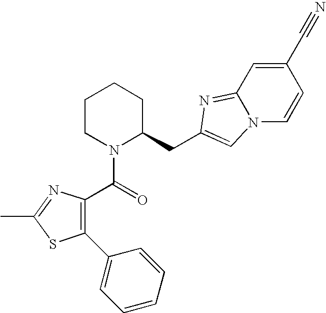

A mixture of 2-methyl-5-phenyl-1,3-thiazole-4-carboxylic acid (0.084 g, 0.38 mmol), DMF (1 ml), DIPEA (0.33 ml, 1.92 mmol) and TBTU (0.15 g, 0.46 mmol) was stirred at room temperature for 20 min. A solution of 2-[(2S)-2-piperidinylmethyl]imidazo[1,2-a]pyridine-7-carbonitrile D15 (0.074 g, 0.31 mmol) in DMF (1 ml) was added and the mixture was stirred for 30 min. The reaction mixture was quenched with brine and extracted with EtOAc. The organic phase was washed with water, dried (Na₂SO₄) and the solvent removed. The crude was purified by chromatography on silica gel (Flash master, DCM/MeOH from 100/0 to 80/20). Collected fractions gave the title compound E9 (0.065 g, 0.15 mmol, 48% yield). MS: (ES/+) m/z: 442 (M+1). C₂₅H₂₃N₅OS requires 441. HPLC (walk-up): rt=3.75 min.

¹H NMR [the product is present as a mixture of conformers (ratio ca. 50/50) and the assignment refers to the single conformer] (500 MHz, CDCl₃) δ (ppm): 8.11 (d, 1H), 7.85 (s, 1H), 7.42 (s, 1H), 7.43-7.38 (m, 2H), 7.25 (t, 1H), 7.19 (t, 2H), 6.89 (d, 1H), 5.25-5.38 (m, 1H), 4.73 (d, 1H), 3.31 (dd, 1H), 3.09 (dd, 1H), 2.93 (dt, 1H), 2.70 (s, 3H), 1.23-1.79 (m, 5H), 0.87-1.01 (m, 1H).

### Example 10

### 6-bromo-7,8-dimethyl-2-({(2S)-1-[(2-methyl-5-phenyl-1,3-thiazol-4-yl)carbonyl]-2-piperidinyl}methyl)imidazo[1,2-a]pyridine (E10)

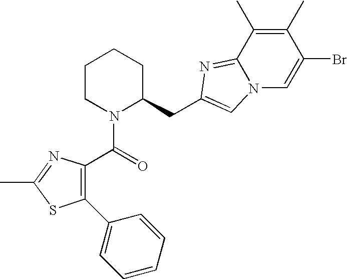

A mixture of 2-methyl-5-phenyl-1,3-thiazole-4-carboxylic acid (0.074 g, 0.34 mmol), DMF (3 ml), DIPEA (0.29 ml, 1.68 mmol) and TBTU (0.13 g, 0.40 mmol) was stirred at room temperature for 20 min. 6-Bromo-7,8-dimethyl-2-[(2S)-2-piperidinylmethyl]imidazo[1,2-a]pyridine D16 (0.090 g, 0.28 mmol) dissolved in DMF (1 ml) was added and the mixture stirred for 2 h. The reaction mixture was diluted with brine (3 ml), extracted with EtOAc (2×4 ml) and the combined organic layers were washed with brine/ice (6×3 ml). The resulting crude was purified by flash chromatography on silica gel (Biotage SP1 12M, DCM/MeOH 95/5). Collected fractions gave the title compound E10 (0.051 g, 0.10 mmol, 35% yield) as a yellow solid. MS: (ES/+) m/z: 523 (M+1, 100%) and 525 (M+1, 100%). C₂₆H₂₇BrN₄OS requires 522. ¹H NMR [the product is present as a mixture of conformers (ratio ca. 50/50) and the assignment refers to a single conformer] (500 MHz, CDCl₃) δ (ppm): 8.07-8.12 (m, 1H), 7.50-7.57 (m, 1H), 7.37 (d, 2H), 7.23-7.29 (m, 1H), 7.18 (t, 2H), 5.26-5.41 (m, 1H), 4.72 (dd, 1H), 3.28-3.38 (m, 1H), 3.05-3.08 (m, 1H), 2.94 (dt, 1H), 2.72 (s, 3H), 2.63 (s, 3H), 2.42 (s, 3H), 1.35-1.76 (m, 5H), 0.99-1.09 (m, 1H).

### Example 11

### 2-({(2S)-1-[(2-methyl-5-phenyl-1,3-thiazol-4-yl)carbonyl]-2-piperidinyl}methyl)-5-(trifluoromethyl)imidazo[1,2-a]pyridine (E1 l)

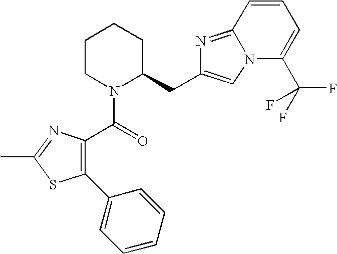

A mixture of 2-methyl-5-phenyl-1,3-thiazole-4-carboxylic acid (0.056 g, 0.25 mmol), DMF (3 ml), DIPEA (0.22 ml, 1.27 mmol) and TBTU (0.098 g, 0.31 mmol) was stirred at room temperature. After 20 min 2-[(2S)-2-piperidinylmethyl]-5-(trifluoromethyl)imidazo[1,2-35a]pyridine D17 (0.060 g, 0.21 mmol) dissolved in DMF (3 ml) was added and the mixture left under stirring overnight. The reaction crude was purified by chromatography on silica gel (Flash Master, DCM/MeOH from 100/0 to 90/10). Collected fractions gave the title compound E11 (0.020 g, 0.04 mmol, 19% yield). MS: (ES/+) m/z: 485 (M+1). C₂₅H₂₃F₃N₄OS requires 484.

¹H NMR [the product is present as a mixture of conformers (ratio ca. 50/50) and the assignment refers to the single conformer] (500 MHz, CDCl₃) δ (ppm): 7.85-7.91 (m, 1H), 7.35-7.47 (m, 2H), 7.16-7.34 (m, 6H), 5.26-5.47 (m, 1H), 4.76 (dd, 1H), 3.11-3.27 (m, 1H), 2.84-3.09 (m, 2H), 2.72 (s, 3H), 1.36-1.94 (m, 5H), 0.83-1.07 (m, 1H).

### Example 12

### 6-bromo-5-methyl-2-({(2S)-1-[(2-methyl-5-phenyl-1,3-thiazol-4-yl)carbonyl]-2-piperidinyl}methyl)imidazo[1,2-a]pyridine (E12)

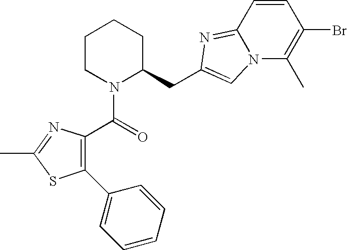

A mixture of 2-methyl-5-phenyl-1,3-thiazole-4-carboxylic acid (0.074 g, 0.34 mmol), DMF (3 ml), DIPEA (0.30 ml, 1.70 mmol) and TBTU (0.13 g, 0.41 mmol) was stirred at room temperature. After 20 min 6-bromo-5-methyl-2-[(2S)-2-piperidinylmethyl]imidazo[1,2-a]pyridine D18 (0.087 g, 0.28 mmol) dissolved in DMF (1 ml) was added and the mixture left under stirring for 6 h. The reaction mixture was diluted with brine (3 ml), extracted with EtOAc (2×4 ml) and the combined organic layers washed with brine/ice (6×3 ml). The reaction crude was purified by flash chromatography on silica gel (Biotage SP1 12M, DCM/MeOH 95/5). Collected fractions gave the title compound E12 (0.003 g, 0.005 mmol, 2% yield) as a yellow solid. MS: (ES/+) m/z: 509 (M+1, 100%) and 510 (M+1, 100%). C₂₅H₂₅BrN₄OS requires 508. ¹H NMR [the product is present as a mixture of conformers (ratio ca. 55/45) and the assignment refers to the minor conformer] (500 MHz, CDCl₃) δ (ppm): 7.64 (s, 1H), 7.19-7.37 (m, 4H), 7.15-7.17 (m, 1H), 7.09 (t, 2H), 4.72 (dd, 1H), 3.91-4.02 (m, 1H), 3.20 (dd, 1H), 2.94-2.99 (m, 1H), 2.66-2.71 (m, 4H), 2.32 (s, 3H), 1.31-1.77 (m, 5H), 0.70-0.80 (m, 1H).

### Example 13

### 8-fluoro-2-({(2S)-1-[(2-methyl-5-phenyl-1,3-thiazol-4-yl)carbonyl]-2-piperidinyl}methyl)imidazo[1,2-a]pyridine (HCl salt) (E13)

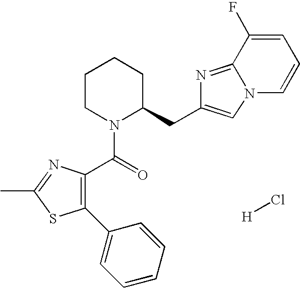

To a mixture of 2-methyl-5-phenyl-1,3-thiazole-4-carboxylic acid (20.39 g, 93 mmol) and DMF (0.327 ml, 4.23 mmol) in DCM (350 ml), oxalyl chloride (18.50 ml, 211 mmol) was added dropwise at 0° C. under Argon atmosphere and the resulting mixture was left under stirring for 45 min at room temperature. The solvent was removed under reduced pressure and the resulting orange solid was dissolved in DCM (250 ml) [acyl chloride solution]. TEA (70.70 ml, 507 mmol) was added at 0° C. to a suspension of 8-fluoro-2-[(2S)-2-piperidinylmethyl]imidazo[1,2-a]pyridine hydrochloride salt D58 (22.80 g) in DCM (350 ml) and the mixture was stirred at 0° C. under Argon atmosphere for 10 min. The acyl chloride solution was added dropwise at 0° C. and the resulting reaction was left under stirring for 1.5 h at room temperature under Argon atmosphere. The mixture was diluted with a saturated NaHCO₃ aqueous solution (600 ml). The organic phase was separated and washed with a saturated NaHCO₃ aqueous solution (2×500 ml) and dried (Na₂SO₄). The solvent was removed under vacuum. The residue was purified via flash chromatography on silica gel (Biotage 75 L, from EtOAc 100 to EtOAC 100/MeOH 0.5). Collected fractions gave the free base of the title compound (23.80 g, 54.80 mmol, 41% yield from D2, three steps). 3H NMR [the compound is present as a mixture of conformers (ratio ca. 55/45), only one assigned] (400 MHz, DMSO-d₆) δ (ppm): 8.28 (d, 1H), 7.65 (d, 1H), 7.21-7.40 (m, 5H), 6.97 (dd, 1H), 6.71-6.77 (m, 1H), 4.46 (bd, 1H), 3.88-4.00 (m, 1H), 2.97-3.14 (m, 2H), 2.75 (dd, 1H), 2.69 (s, 3H), 0.91-1.74 (m, 6H).

This material was combined with 0.70 g of a batch coming from an identical reaction carried out on 0.90 g (3.34 mmol) of D58. The free base (24.50 g, 56.40 mmol) was suspended in diethyl ether (500 ml) and the mixture cooled to 0° C. and stirred under Argon atmosphere for 15 min. HCl (33.80 ml of a 2 M solution in Et₂O, 67.70 mmol) was added dropwise at 0 C. and the mixture was stirred for 1.5 h at room temperature. Volatiles were removed under reduced pressure.

The resulting solid was triturated with Et₂O (3×1 L) and then dried overnight under vacuum at 40° C. to afford the title compound E13 (21.50 g, 45.60 mmol, 34% from D2, four steps). MS: (ES/+) m/z: 435 (M+1−HCl). C₂₄H₂₄ClFN₄OS requires 470.

### Example 14

### 2-[((2S)-1-{[5-(4-fluorophenyl)-2-methyl-1,3-thiazol-4-yl]carbonyl}-2-piperidinyl)methyl]-8-methylimidazo[1,2-a]pyridine (E14)

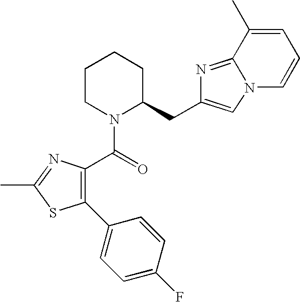

To a solution of 5-(4-fluorophenyl)-2-methyl-1,3-thiazole-4-carboxylic acid (0.021 g, 0.09 mmol) in DCM (1 ml), oxalyl chloride (0.017 ml, 0.19 mmol) and dry DMF (0.006 ml, 0.09 mml) were added. The mixture was left under stirring for 1 h and then concentrated under vacuum to provide the acyl chloride that was dissolved in DCM (1 ml). The acyl chloride solution was added to an ice-cooled mixture of 8-methyl-2-[(2S)-2-piperidinylmethyl]imidazo[1,2-a]pyridine D12 (0.020 g, 0.09 mmol) and TEA (0.04 ml, 0.26 mmol) in DCM (1 ml). The reaction mixture was left under stirring at room temperature for 2 h, diluted with DCM and washed with a saturated NaHCO₃ aqueous solution and brine. The organic layer was dried (Na₂SO₄), filtered and the solvent removed under vacuum to give the title compound E14 (0.039 g, 0.08 mmol, 95% yield) as a grey solid. MS: (ES/+) m/z: 449 (M+1). C₂₅H₂₅FN₄OS requires 448. UPLC: rt=2.23 min, peak observed: 449 (M+1).

¹H NMR [the product is present as a mixture of conformers (ratio ca. 55/45) and the assignment refers to the minor component] (500 MHz, CDCl₃) δ (ppm): 7.89 (d, 1H), 7.64 (s, 1H), 7.24-7.31 (m, 2H), 6.88-6.95 (m, 1H), 6.78 (t, 2H), 6.60-6.67 (m, 1H), 5.34-5.41 (m, 1H), 3.27-3.38 (m, 2H), 3.02-3.13 (m, 2H), 2.71 (s, 3H), 2.60 (s, 3H), 1.31-1.77 (m, 5H), 1.08-1.20 (m, 1H).

The following compounds of formula (IV), where R represents a single substitution with R₂ or a substitution with R₂ and R₃, examples 15 to 21, were prepared using a similar procedure to that described for Example 14. Each compound was obtained by amide coupling of the appropriate piperidine with 5-(4-fluorophenyl)-2-methyl-1,3-thiazole-4-carboxylic acid.

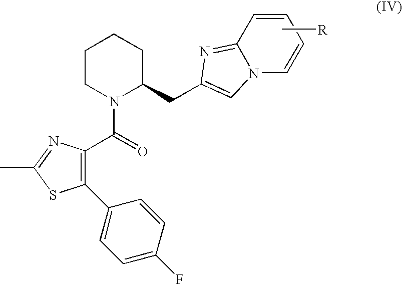

The compounds of examples 15 to 21 are as follows:

### Example 15 (E15)

2-[((2S)-1-{[5-(4-fluorophenyl)-2-methyl-1,3-thiazol-4-yl]carbonyl}-2-piperidinyl)methyl]-8-(trifluoromethyl)imidazo[1,2-a]pyridine (HCl salt);

### Example 16 (E16)

6,8-difluoro-2-[((2S)-1-{[5-(4-fluorophenyl)-2-methyl-1,3-thiazol-4-yl]carbonyl}-2-piperidinyl)methyl]imidazo[1,2-a]pyridine (HCl salt);

### Example 17 (E17)

6,8-dichloro-2-[((2S)-1-{[5-(4-fluorophenyl)-2-methyl-1,3-thiazol-4-yl]carbonyl}-2-piperidinyl)methyl]imidazo[1,2-a]pyridine (HCl salt);

### Example 18 (E18)

6-fluoro-2-[((2S)-1-{[5-(4-fluorophenyl)-2-methyl-1,3-thiazol-4-yl]carbonyl}-2-piperidinyl)methyl]imidazo[1,2-a]pyridine;

### Example 19 (E19)

2-[((2S)-1-{[5-(4-fluorophenyl)-2-methyl-1,3-thiazol-4-yl]carbonyl}-2-piperidinyl)methyl]imidazo[1,2-a]pyridine-7-carbonitrile (HCl salt);

### Example 20 (E20)

2-[((2S)-1-{[5-(4-fluorophenyl)-2-methyl-1,3-thiazol-4-yl]carbonyl}-2-piperidinyl)methyl]-7-(methyloxy)imidazo[1,2-a]pyridine (HCl salt);

### Example 21 (E21)

2-[((2S)-1-{[5-(4-fluorophenyl)-2-methyl-1,3-thiazol-4-yl]carbonyl}-2-piperidinyl)methyl]imidazo[1,2-a]pyridine-8-carbonitrile (HCl salt).

| | Piperidine | |
|---|---|---|
| | starting | |
| No. | material | Characterising data |
| 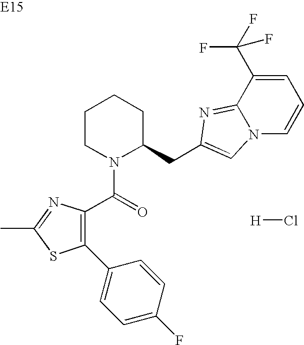 | D8 | Free base:UPLC: rt = 0.71 min, peak observed: 503 (M + 1). C₂₅H₂₂F₄N₄OSrequires 502.HCl salt:UPLC: rt = 0.70 min, peak observed: 503 (M + 1 − HCl).C₂₅H₂₃ClF₄N₄OS requires 538. ¹H NMR [the product is presentas a mixture of conformers (ratio ca. 60/40), only one assigned](500 MHz, DMSO-d₆) δ(ppm):8.94-9.07(m, 1 H), 8.22(s, 1 H),8.03-8.18(m, 1 H), 7.24-7.57(m, 1 H), 7.05-7.47(m, 4 H),5.10-5.29(m, 1 H), 4.46(d, 1 H), 3.10-3.25(m, 3 H), 2.64(s, 3 H), 0.85-1.77(m, 6 H). |
| 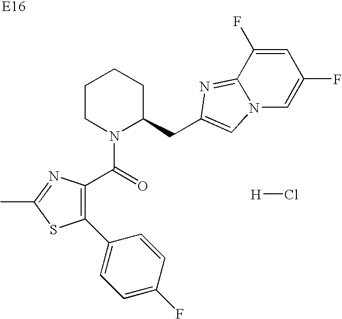 | D13 | Free base:UPLC: rt = 0.69 min, peak observed: 471 (M + 1). C₂₄H₂₁F₃N₄OSrequires 470.HCl salt:UPLC: rt = 0.69 min, peak observed: 471 (M + 1 − HCl).C₂₄H₂₂ClF₃N₄OS requires 506.¹H NMR ([the compound is present as a mixture of conformers(ratio ca. 60/40), only one assigned] (500 MHz, DMSO-d₆)δ(ppm):8.83-8.90(m, 1 H), 8.05-8.10(m, 1 H), 7.75-7.90(m, 1 H), 7.09-7.47(m, 4 H), 5.08-5.23(m, 1 H), 4.46(d, 1 H),2.99-3.25(m, 3 H), 2.67(s, 3 H), 0.74-1.92(m, 6 H). |
| 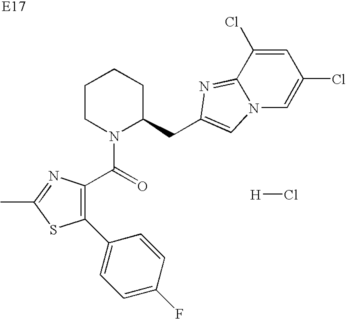 | D10 | Free base:UPLC: rt = 0.76 min, peaks observed: 503 (M + 1, 100%), and 505(M + 1, 66%). C₂₄H₂₁Cl₂FN₄OS requires 502.HCl salt:UPLC: rt = 0.75 min, peaks observed: 503 (M + 1 − HCl, 100%)and 505 (M + 1 − HCl, 66%). C₂₄H₂₂Cl₃FN₄OS requires 538.¹H NMR [the compound is present as a mixture ofconformers(ratio ca. 60/40), only one assigned] (500 MHz, DMSO-d₆)δ(ppm): 8.88(s, 1 H), 8.01(s, 1 H), 7.83(s, 1 H), 7.15-7.21(m, 2 H), 7.06-7.13(m, 2 H0, 4.47(dd, 1 H), 4.01-4.09(m, 1 H),3.34-3.47(m, 1 H), 3.04-3.22(m, 1 H0, 2.63-2.73(m, 1 H),2.49(s, 3 H), 1.08-1.79(m, 6 H). |
| 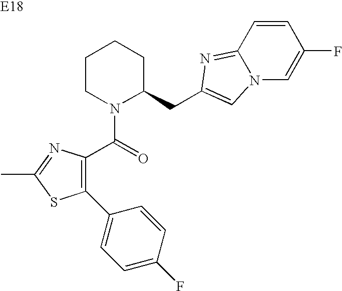 | D14 | Free base:MS: (ES/+) m/z: 453 (M + 1). C₂₄H₂₂F₂N₄OS requires 452.UPLC: rt = 0.57 min, peak observed: 453 (M + 1).¹H NMR [the compound is present as a mixture of conformers(ratio ca. 50/50), only one assigned] (500 MHz, CDCl₃) δ(ppm):7.94-7.99(m, 1 H), 7.65(s, 1 H), 7.51(dd, 1 H), 7.31-7.39(m,2 H), 7.00-7.14(m, 1 H), 6.89(t, 2 H),5.21-5.47(m, 1 H),4.74(d, 1 H), 2.87-3.38(m, 3 H), 2.37(s, 3 H), 0.79-1.84(m, 6 H). |
| 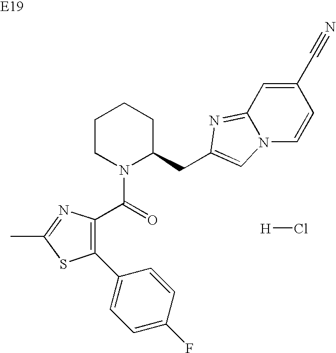 | D15 | Free base:UPLC: rt = 0.65 min, peak observed: 460 (M + 1). C₂₅H₂₂FN₅OSrequires 459. ¹H NMR [the compound is present as a mixture ofconformers (ratio ca. 55/45). Assignment is provided for onerotamer] (500 MHz, DMSO-d₆) δ(ppm): 8.57(d, 1 H), 8.01-8.05(m, 1 H), 7.77(s, 1 H), 7.02-7.49(m, 5 H), 5.11-5.20(m,1 H), 4.48(d, 1 H), 2.72-3.26(m, 3 H), 2.68(s,3 H), 0.77-1.86(m, 6 H).HCl salt:MS: (ES/+) m/z: 460 (M + 1 − HCl). C₂₅H₂₃ClFN₅OS requires 495.HPLC (walk-up): rt = 3.84 min. |
| 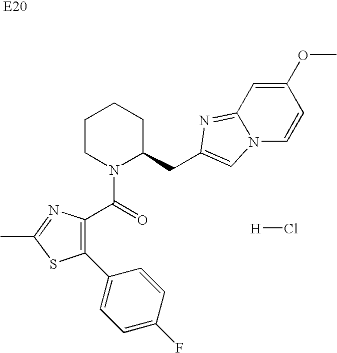 | D20 | Free base:HPLC (walk-up): rt = 3.78 min. ¹H NMR [the compound ispresent as a mixture of conformers (ratio ca. 52/48) and theassignment refers to the major component] (500 MHz, DMSO-d₆) δ(ppm): 8.21(d, 1 H), 7.31(s, 1 H), 7.26(dd, 2 H), 7.15(t, 2 H),6.66(d, 1 H), 6.47(dd, 1 H), 4.45(dd, 1 H), 3.89-3.98(m, 1 H),3.75(s, 3 H), 2.97-3.13(m, 2 H), 2.77-2.86(m, 1 H), 2.59(s, 3 H),0.95-1.76(m, 6 H).HCl salt:UPLC: rt = 0.59 min, peak observed: 465 (M + 1 − HCl).C₂₅H₂₆ClFN₄O₂S requires 500. |
| 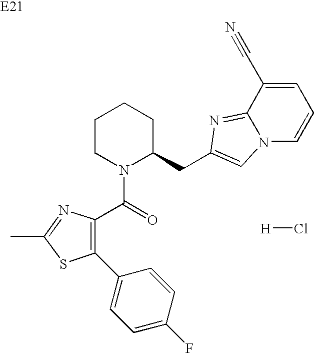 | D21 | Free base:UPLC: rt = 0.67 min, peak observed: 460 (M + 1). C₂₅H₂₂FN₅OSrequires 459. ¹H NMR [the compound is present as a mixture ofconformers (ratio ca. 60/40) and the assignment refers to themajor component] (500 MHz, DMSO-d₆) δ(ppm): 8.72(d, 1 H),7.75-7.80(m, 2 H), 7.13-719(m, 2 H), 7.07(t, 2 H), 6.93(t, 1 H),4.45(d, 1 H), 4.03-4.11(m, 1 H), 3.21(dd,1 H), 3.06(dt, 1 H),2.75(dd, 1 H), 2.67-2.70(m, 3 H), 0.96-1.78(m, 6 H).HCl salt:UPLC: rt = 0.67 min, peak observed: 460 (M + 1 − HCl).C₂₅H₂₃ClFN₅OS requires 495. |

### Example 22

### 5-fluoro-2-({(2S)-1-[(2-methyl-5-phenyl-1,3-thiazol-4-yl)carbonyl]-2-piperidinyl}methyl)imidazo[1,2-a]pyridine (E22)

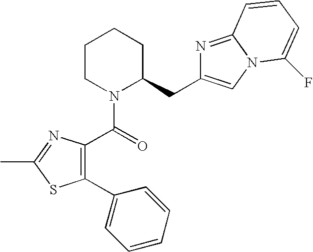

To a solution of 2-methyl-5-phenyl-1,3-thiazole-4-carboxylic acid (0.021 g, 0.09 mmol) in DCM (1 ml), oxalyl chloride (0.018 ml, 0.21 mmol) and then DMF (0.007 ml, 0.09 mmol) were added and the resulting mixture was stirred for 30 min. The solvent was removed under reduced pressure, the resulting yellow solid was dissolved in DCM (1 ml) and the acyl chloride solution was added dropwise to an ice-cooled mixture of 5-fluoro-2-[(2S)-2-piperidinylmethyl]imidazo[1,2-a]pyridine D22 (0.020 g, 0.09 mmol) and TEA (0.04 ml, 0.26 mmol) in DCM (1 ml). The mixture was allowed to warm up to room temperature and stirred for 1 h. The reaction mixture was then diluted with DCM (1 ml) and washed with a saturated NaHCO₃ aqueous solution (2 ml). The organic phase was separated, dried (Na₂SO₄), filtered and concentrated. The residue was purified by flash chromatography on silica gel (Biotage 12 M, DCM/MeOH 98/2). The title compound E22 (0.014 g, 0.03 mmol, 34% yield) was obtained as a yellow solid. ¹H NMR [the product is present as a mixture of conformers (ratio ca. 55/45), only one assigned] (500 MHz, CDCl₃) δ (ppm): 7.36-7.45 (m, 3H), 7.23-7.32 (m, 4H), 7.12-7.22 (m, 1H), 6.38-6.47 (m, 1H), 4.76 (dd, 1H), 4.00-4.07 (m, 1H), 2.92-3.24 (m, 2H), 2.79 (dd, 1H), 2.41 (s, 3H), 1.27-1.80 (m, 4H), 0.75-1.05 (m, 2H).

### Example 23

### 3-methyl-2-({(2S)-1-[(2-methyl-5-phenyl-1,3-thiazol-4-yl)carbonyl]-2-piperidinyl}methyl)imidazo[1,2-a]pyridine (E23)

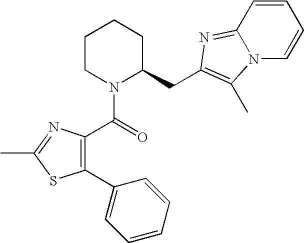

To a solution of 2-methyl-5-phenyl-1,3-thiazole-4-carboxylic acid (0.00526 g, 0.024 mmol) in DCM (0.33 ml), oxalyl chloride (0.00462 ml, 0.053 mmol) and then DMF (0.001688 ml, 0.022 mmol) were added and the resulting mixture stirred for 30 min. The solvent was removed under reduced pressure, the resulting yellow solid was dissolved in DCM (0.33 ml) and the acyl chloride solution was added dropwise at 0° C. to a mixture of 3-methyl-2-[(2S)-2-piperidinylmethyl]imidazo[1,2-a]pyridine D27 (0.005 g, 0.022 mmol) and TEA (0.00912 ml, 0.065 mmol) in DCM (0.33 ml). The mixture was left under stirring at room temperature for 1 h, then diluted with DCM (1 ml) and washed with a saturated NaHCO₃ aqueous solution (2 ml). The organic phase was separated, dried (Na₂SO₄), filtered and concentrated. The residue was purified by flash chromatography on silica gel (Biotage 12 M, DCM/MeOH 98/2). Collected fractions gave the title compound E23 (0.008 g, 0.015 mmol, 68.2% yield) as a yellowish solid. MS: (ES/+) m/z: 431 (M+1). C₂₅H₂₆N₄OS requires 430.

¹H NMR [the compound is present as a mixture of conformers (ratio ca. 60-40), only one assigned] (500 MHz, CDCl₃) δ(ppm): 7.81 (d, 1H), 7.55 (d, 1H), 7.29-7.50 (m, 5H), 7.08-7.17 (m, 1H), 6.77-6.85 (m, 1H), 4.73 (d, 1H), 3.92-4.02 (m, 1H), 3.16 (dd, 1H), 2.99-3.07 (m, 1H), 2.67-2.78 (m, 4H), 2.21 (s, 3H), 1.25-1.78 (m, 5H), 0.72-0.84 (m, 1H).

### Example 24

### 3-iodo-2-({(2S)-1-[(2-methyl-5-phenyl-1,3-thiazol-4-yl)carbonyl]-2-piperidinyl}methyl)imidazo[1,2-a]pyridine (HCl salt) (E24)

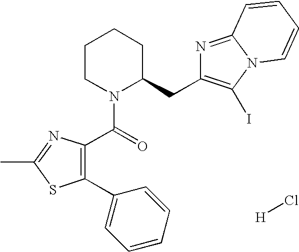

To a solution of 2-methyl-5-phenyl-1,3-thiazole-4-carboxylic acid (0.010 g, 0.05 mmol) in DCM (1 ml), oxalyl chloride (0.009 ml, 0.10 mmol) and then DMF (0.003 ml, 0.04 mmol) were added and the resulting mixture was stirred for 30 min. The solvent was removed under reduced pressure, the resulting yellow solid was dissolved in DCM (1 ml) and added dropwise at 0° C. to a mixture of 3-iodo-2-[(2S)-2-piperidinylmethyl]imidazo[1,2-a]pyridine D25 (0.014 g, 0.04 mmol) and TEA (0.02 ml, 0.12 mmol) in DCM (1 ml). The mixture was warmed up to room temperature and left under stirring for 1 h, then diluted with a little amount of DCM and washed with a saturated NaHCO₃ aqueous solution (2 ml). The organic phase was separated, dried (Na₂SO₄), filtered and concentrated. The residue was purified by flash chromatography on silica gel (Biotage 12 M, DCM/MeOH 98/2). Collected fractions gave the free base of the title compound (0.017 g, 0.03 mmol, 70% yield) as a light brown solid. UPLC: rt=0.62 min, peak observed: 543 (M+1). C₂₄H₂₃IN₄OS requires 542. ¹H NMR [the compound is present as a mixture of conformers (ratio ca. 65/35), only one assigned] (500 MHz, DMSO-d₆) δ (ppm): 8.18 (d, 1H), 7.47 (d, 1H), 7.21-7.41, (m, 6H), 6.99 (t, 1 H), 4.47 (dd, 1H), 3.88-3.98 (m, 1H), 2.99-3.20 (m, 2H), 2.76 (dd, 1H), 2.39 (s, 3H), 1.11-1.78 (m, 5H), 0.70-0.98 (m, 1H).

The free base (0.015 g, 0.03 mmol) was dissolved in anhydrous DCM (1 ml) and the solution was cooled to 0° C. A 1 M HCl solution in Et₂O (0.03 ml, 0.03 mmol) was added and the mixture left under stirring for 15 min. The solvent was removed under reduced pressure and the resulting solid triturated with anhydrous Et₂O, giving the title compound E24 (0.016 g, 0.02 mmol, 83% yield) as a light brown solid. UPLC: rt=0.63 min, peak observed: 543 (M+1-HCl). C₂₄H₂₄ClIN₄OS requires 578.

### Example 25

### 3-chloro-2-({(2S)-1-[(2-methyl-5-phenyl-1,3-thiazol-4-yl)carbonyl]-2-piperidinyl}methyl)imidazo[1,2-a]pyridine (HCl salt) (E25)

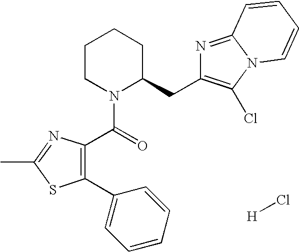

To a solution of 2-methyl-5-phenyl-1,3-thiazole-4-carboxylic acid (0.026 g, 0.12 mmol) in DCM (0.50 ml), oxalyl chloride (0.023 ml, 0.26 mmol) and then DMF (0.009 ml, 0.12 mmol) were added and the resulting mixture was stirred at room temperature for 30 min. The acyl chloride solution was added dropwise at 0° C. to a mixture of 3-chloro-2-[(2S)-2-piperidinylmethyl]imidazo[1,2-a]pyridine D28 (0.030 g, 0.12 mmol) and TEA (0.05 ml, 0.36 mmol) in DCM (1 ml). The mixture was allowed to warm up to room temperature and left under stirring for 1 h. The reaction mixture was then diluted with DCM (2 ml) and washed with a saturated NaHCO₃ aqueous solution (2×3 ml). The two phases were separated and the organic one dried (Na₂SO₄), filtered and concentrated. The residue was purified by chromatography on silica gel (Vac Master 10 g, EtOAc and then DCM/MeOH 95/5). Collected fractions gave the free base of the title compound (0.031 g, 0.06 mmol, 53% yield) as a yellow solid. UPLC: rt=0.69 min, peaks observed: 451 (M+1, 100%) and 453 (M+1, 33%). C₂₄H₂₃ClN₄OS requires 450.

¹H NMR [the compound is present as a mixture of conformers (ratio ca. 65/35), only one assigned] (500 MHz, DMSO-d₆) δ (ppm): 8.18 (d, 1H), 7.47 (d, 1H), 7.21-7.41 (m, 6H), 6.99 (t, 1H), 4.47 (dd, 1H), 3.88-3.98 (m, 1H), 2.99-3.20 (m, 2H), 2.76 (dd, 1H), 2.39 (s, 3H), 1.11-1.78 (m, 5H), 0.70-0.98 (m, 1H).

To a solution of the free base (0.031 g, 0.06 mmol) in DCM (1 ml), a 1 M HCl solution (0.10 ml, 0.100 mmol) was added the mixture left under stirring for 30 min. Volatiles were removed under reduced pressure to give the title compound E25 (0.034 g, 0.02 mmol, 95% yield). LC-MS: rt=1.90 min, peaks observed 451 (M+1-HCl, 100%) and 453 (M+1-HCl, 33%). C₂₄H₂₄Cl₂N₄OS requires 486.

### Example 26

### 3-chloro-2-({(2S)-1-[(2-methyl-5-phenyl-1,3-thiazol-4-yl)carbonyl]-2-piperidinyl}methyl)-7-(trifluoromethyl)imidazo[1,2-a]pyridine (HCl salt) (E26)

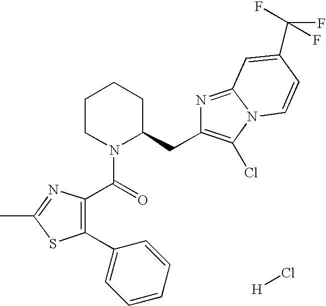

To a solution of 2-methyl-5-phenyl-1,3-thiazole-4-carboxylic acid (0.018 g, 0.08 mmol) in DCM (1 ml), oxalyl chloride (0.016 ml, 0.18 mmol) and then DMF (0.006 ml, 0.008 mmol) were added and the resulting mixture was stirred for 30 min. The solvent was removed under reduced pressure, the resulting yellow solid was dissolved in DCM (1 ml) and added dropwise at 0° C. to a solution of 3-chloro-2-[(2S)-2-piperidinylmethyl]-7-(trifluoromethyl)imidazo[1,2-a]pyridine D30 (0.024 g, 0.08 mmol) and TEA (0.04 ml, 0.23 mmol) in DCM (1 ml). The mixture was left under stirring at room temperature for 1 h, then diluted with DCM, washed with a saturated NaHCO₃ aqueous solution, separated, dried (Na₂SO₄), filtered and concentrated. The residue was purified by column chromatography on silica gel (Biotage 12M, Cy/EtOAc 50/50). Collected fractions gave the free base of the title compound (0.015 g, 0.03 mmol, 36% yield) as a yellowish solid. UPLC: rt=0.84 min, peaks observed: 519 (M+1, 100%) and 521 (M+1, 33%). C₂₅H₂₂ClF₃N₄O requires 518. ¹HNMR [the compound is present as a mixture of conformers (ratio ca. 70/30) and the assignment refers to the major component] (500 MHz, DMSO-d₆) δ(ppm): 8.39 (d, 1H), 7.96 (s, 1H), 7.14-7.42 (m, 6H), 4.48 (dd, 1H), 3.89-3.98 (m, 1H), 3.23 (dd, 1H), 3.07 (t, 1H), 2.72 (dd, 1H), 2.34 (s, 3H), 0.71-1.77 (m, 6H). The free base (0.014 g, 0.026 mmol) was dissolved in DCM (1 ml) and the solution cooled to 0° C. A 1 M HCl solution in Et₂O (0.04 ml, 0.04 mmol) was added at 0° C. and the mixture left under stirring for 15 min. The solvent was removed under reduced pressure and the resulting solid triturated with anhydrous Et₂0 to give the title compound E26 (0.014 g, 0.023 mmol, 90% yield) as a light brown solid. HPLC (walk-up): rt=5.55 min. MS: (ES/+) m/z: 519 (M+1−HCl). C₂₅H₂₃Cl₂N₄O requires 554.

### Example 27

### 3-fluoro-8-methyl-2-({(2S)-1-[(2-methyl-5-phenyl-1,3-thiazol-4-yl)carbonyl]-2-piperidinyl}methyl)imidazo[1,2-a]pyridine (HCl salt) (E27)

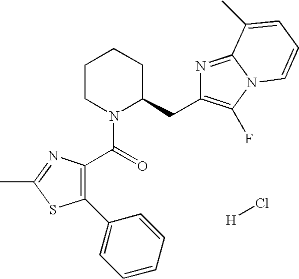

To a solution of 2-methyl-5-phenyl-1,3-thiazole-4-carboxylic acid (0.01365 g, 0.062 mmol) in DCM (1 ml), oxalyl chloride (0.012 ml, 0.137 mmol) and then DMF (0.00438 ml, 0.057 mmol) were added and the resulting mixture was stirred for 30 min. The solvent was removed under reduced pressure and the resulting yellow solid was dissolved in DCM (1 ml). The acyl chloride solution was added dropwise at 0° C. to a mixture of 3-fluoro-8-methyl-2-[(2S)-2-piperidinylmethyl]imidazo[1,2-a]pyridine (0.014 g, 0.057 mmol) D31 and TEA (0.024 ml, 0.17 mmol) in DCM (1 ml). The mixture was allowed to warm up to room temperature and left under stirring for 1 h. The reaction mixture was then diluted with DCM (5 ml) and washed with a saturated NaHCO₃ aqueous solution (2 ml). The two phases were separated, dried (Na₂SO₄), filtered and concentrated. The residue was purified by flash chromatography on silica gel (Biotage 12 M, Cy/EtOAc 50/50). Collected fractions gave the free base of the title compound (0.0113 g, 0.025 mmol, 44.1% yield) as a yellow oil.

HPLC (walk-up): rt=3.75 min. MS: (ES/+) m/z: 449 (M+1). UPLC: rt=0.62 min, peak observed: 449 (M+1). C₂₅H₂₅FN₄OS requires 448.

¹H NMR [the compound is present as a mixture of conformers (ratio ca. 65/35), only one assigned] (500 MHz, DMSO-d₆) δ(ppm): 7.98 (d, 1H), 7.19-7.47 (m, 5H), 6.93 (d, 1H), 6.80 (t, 1H), 4.45-4.52 (m, 1H), 3.85-3.93 (m, 1H), 3.09-3.20 (m, 1H), 3.00 (td, 1H), 2.60-2.71 (m, 4H), 2.33 (s, 3H), 0.75-1.74 (m, 6H).

The free base (0.0113 g, 0.025 mmol) was dissolved in DCM (1 ml) and the solution cooled to 0° C. A 1 M HCl solution in Et₂O (0.038 ml, 0.038 mmol) was added and the mixture left under stirring for 15 min. The solvent was removed under reduced pressure and the resulting solid triturated with anhydrous Et₂O, giving the title compound E27 (0.0117 g, 0.024 mmol, 95% yield) as a brown solid. HPLC (walk-up): rt=3.75 min. MS: (ES/+) m/z: 449 (M+1−HCl). UPLC: rt=0.62 min, peak observed: 449 (M+1−HCl). C₂₅H₂₆ClFN₄OS requires 484.

### Example 28

### 3-chloro-6-fluoro-2-({(2S)-1-[(2-methyl-5-phenyl-1,3-thiazol-4-yl)carbonyl]-2-piperidinyl}methyl)imidazo[1,2-a]pyridine (HCl salt) (E28)

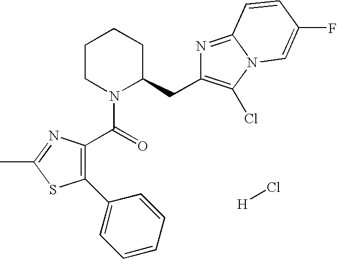

To a solution of 2-methyl-5-phenyl-1,3-thiazole-4-carboxylic acid (0.041 g, 0.19 mmol) in anhydrous DCM (1 ml), oxalyl chloride (0.011 ml, 0.13 mmol) and then DMF (0.02 ml) were added and the resulting mixture stirred at room temperature for 1 h. The solvent was removed under reduced pressure and the residue was dissolved in dry DCM (1 ml). The acyl chloride solution was added dropwise at 0° C. to a mixture of 3-chloro-6-fluoro-2-[(2S)-2-piperidinylmethyl]imidazo[1,2-a]pyridine D33 (0.045 g, 0.17 mmol) and TEA (0.032 ml, 0.23 mmol) in anhydrous DCM and the mixture was stirred at room temperature for 1 h. The reaction mixture was then diluted with a saturated NaHCO₃ aqueous solution and water and extracted with DCM. The organic phase was collected by a phase separator tube and concentrated. The residue was purified by flash chromatography on silica gel (Biotage 12 M, Cy/EtOAc from 100/0 to 50/50). Collected fractions gave the free base of the title compound (0.045 g, 0.10 mmol, 57% yield) as a white solid.

HPLC (walk-up): rt=4.23 min. UPLC: rt=0.75 min, peaks observed: 469 (M+1, 100%) and 471 (M+1, 33%). C₂₄H₂₂ClFN₄OS requires 468. ¹H NMR [the compound is present as a mixture of conformers (ratio ca. 70/30), only one assigned] (500 MHz, DMSO-d₆) δ (ppm): 8.40 (dd, 1H), 7.53 (dd, 1H), 7.19-7.45 (m, 6H), 4.48 (dd, 1H), 3.91-3.99 (m, 1H), 3.01-3.17 (m, 2H), 2.73 (dd, 1H), 2.44 (s, 3H), 0.81-1.74 (m, 6H). The free base (0.045 g, 0.096 mmol) was dissolved in DCM (1 ml) and Et₂O (1 ml), then a 1 M HCl solution in Et₂O (0.11 ml, 0.1 mmol) was added and the mixture left under stirring. After solvent removal and trituration with Et₂O the title compound E28 (0.045 g, 0.09 mmol, 90% yield) was obtained as a white solid. HPLC (walk-up): rt=4.17 min. UPLC: rt=0.75 min, peaks observed: 469 (M+1−HCl, 100%) and 471 (M+1−HCl, 33%). C₂₄H₂₃Cl₂FN₄OS requires 504.

### Example 29

### 8-(methyloxy)-2-({(2S)-1-[(2-methyl-5-phenyl-1,3-thiazol-4-yl)carbonyl]-2-piperidinyl}methyl)imidazo[1,2-a]pyridine (HCl salt) (E29)

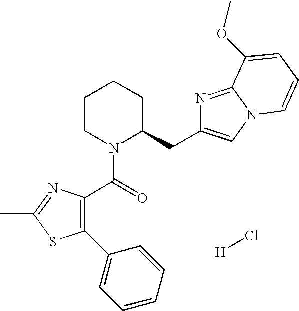

To a solution of 2-methyl-5-phenyl-1,3-thiazole-4-carboxylic acid (0.021 g, 0.10 mmol) in DCM (1 ml), oxalyl chloride (0.020 ml, 0.23 mmol) and then one drop of DMF were added and the resulting mixture was stirred for 1 h. The solvent was removed under reduced pressure and the residue dissolved in DCM. The acyl chloride solution was added dropwise at 0° C. to a mixture of 8-(methyloxy)-2-[(2S)-2-piperidinylmethyl]imidazo[1,2-a]pyridine D35 (0.024 g, 0.10 mmol) and TEA (0.040 ml, 0.29 mmol) in DCM (1 ml). The mixture was left under stirring at room temperature for 2 h, then diluted with DCM and washed with a saturated NaHCO₃ aqueous solution. The organic phase was separated, dried (Na₂SO₄), filtered and concentrated. The residue was purified by chromatography on silica gel (Flash Master, Cy/EtOAc 50/50 and then DCM/MeOH 99/1). Collected fractions gave the free base of the title compound (0.004 g, 0.009 mmol, 9% yield). HPLC (walk-up): rt=3.60 min.

MS: (ES/+) m/z: 447 (M+1). C₂₅H₂₆N₄O₂S requires 446. The free base (0.004 g, 0.009 mmol) was dissolved in DCM (0.50 ml) and Et₂O (0.50 ml) and the solution cooled to 0° C. A 1 M HCl solution in Et₂O (0.019 ml, 0.019 mmol) was added and the mixture left under stirring.

The solvent was removed under reduced pressure and the resulting solid was triturated with Et₂O to give the title compound E29 (0.005 g, 0.009 mmol, 99% yield).

UPLC: rt=0.57 min, peak observed: 447 (M+1−HCl). C₂₅H₂₇ClN₄O₂S requires 482.

### Example 30

### 3-chloro-7-(methyloxy)-2-({(2S)-1-[(2-methyl-5-phenyl-1,3-thiazol-4-yl)carbonyl]-2-piperidinyl}methyl)imidazo[1,2-a]pyridine (HCl salt) (E30)

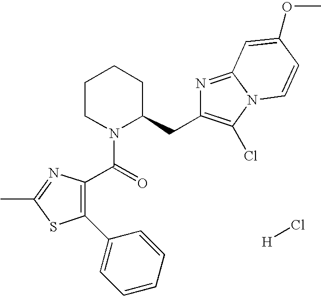

To a solution of 2-methyl-5-phenyl-1,3-thiazole-4-carboxylic acid (0.013 g, 0.06 mmol) in DCM (1 ml), DMF (0.005 ml, 0.06 mmol) was added and the mixture cooled to 0° C. Oxalyl chloride (0.012 ml, 0.13 mmol) was added and the resulting reaction mixture was stirred at room temperature for 30 min. Volatiles were removed under vacuum and the residue dissolved in DCM (1 ml). The acyl chloride solution was added dropwise at 0° C. to a mixture of 3-chloro-7-(methyloxy)-2-[(2S)-2-piperidinylmethyl]imidazo[1,2-a]pyridine D37 (0.017 g, 0.06 mmol) and TEA (0.025 ml, 0.18 mmol) in DCM (1 ml). The reaction mixture was left under stirring at room temperature for 1.5 h then diluted with DCM (2 ml) and washed with a saturated NaHCO₃ aqueous solution (2 ml). The organic phase was separated through a phase separator tube and concentrated. The residue was purified by chromatography on silica gel (Vac Master, EtOAc). Collected fractions gave the free base of the title compound (0.012 g, 0.02 mmol, 36% yield). UPLC: rt=0.75 min, peaks observed: 481 (M+1, 100%) and 483 (M+1, 33%). C₂₅H₂₅ClN₄O₂S requires 480. ¹H NMR [the compound is present as a mixture of conformers (ratio ca. 60/40) and the assignment refers to the major component] (500 MHz, DMSO-d₆) δ(ppm): 8.04 (d, 1H), 7.23-7.43 (m, 5H), 6.86 (s, 1H), 6.70 (d, 1H), 4.47 (d, 1H), 3.88-3.96 (m, 1H), 3.81-3.84 (m, 3H), 2.96-3.05 (m, 2H), 2.72 (dd, 1H), 2.46 (s, 3H), 0.74-1.69 (m, 6H). The free base (0.010 g, 0.021 mmol) was dissolved in DCM (1 ml) and a 1 M HCl solution in Et₂O (0.031 ml, 0.031 mmol) was added. The mixture was left under stirring for 30 min. The solvent was removed under reduced pressure to afford the title compound E30 (0.011 g, 0.019 mmol, 92% yield).

HPLC (walk-up): rt=4.03 min.

### Example 31a

### 6-fluoro-8-methyl-2-({(2S)-1-[(2-methyl-5-phenyl-1,3-thiazol-4-yl)carbonyl]-2-piperidinyl}methyl)imidazo[1,2-a]pyridine (HCl salt) (E31a)

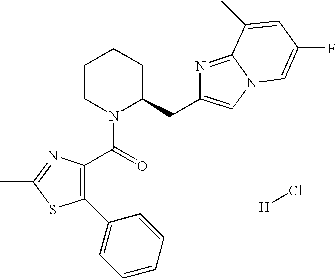

To a solution of 2-methyl-5-phenyl-1,3-thiazole-4-carboxylic acid (0.0195 g, 0.089 mmol) in DCM (1 ml), oxalyl chloride (0.017 ml, 0.196 mmol) and then DMF (0.00626 ml, 0.081 mmol) were added and the resulting mixture stirred for 30 min. The solvent was removed under reduced pressure and the resulting yellow solid was dissolved in DCM (1 ml). The acyl chloride solution was added dropwise at 0° C. to a mixture of 6-fluoro-8-methyl-2-[(2S)-2-piperidinylmethyl]imidazo[1,2-a]pyridine D40a (0.020 g, 0.081 mmol) and TEA (0.034 ml, 0.243 mmol) in DCM (1 ml). The mixture was allowed to warm up to room temperature under stirring for 1 h. The reaction mixture was then diluted with DCM (5 ml) and washed with a saturated NaHCO₃ aqueous solution (2 ml). The two phases were separated and the organic one was dried (Na₂SO₄), filtered and concentrated. The residue was purified by flash chromatography on silica gel (Biotage 12 M, Cy/EtOAc 50/50). Collected fractions gave the free base of the title compound (0.033 g, 0.066 mmol, 82% yield) as a yellow solid.

UPLC: rt=0.58 min, peak observed: 449 (M+1). C₂₅H₂₅FN₄OS requires 448.

¹H NMR [the compound is present as a mixture of 2 conformers (ratio ca. 55/45), only one assigned] (500 MHz, DMSO-d₆) δ (ppm): 8.43 (d, 1H), 7.52 (s, 1H), 7.07-7.44 (m, 5H), 6.96 (d, 1H), 4.32-4.55 (m, 1H), 3.88-4.16 (m, 1H), 3.16-3.24 (m, 1H), 2.93-3.16 (m, 2H), 2.49 (s, 3H), 2.29 (s, 3H), 0.72-1.78 (m, 6H). The free base (0.030 g, 0.067 mmol) was dissolved in DCM anhydrous (1 ml), then a 1 M HCl solution in Et₂O (0.10 ml, 0.10 mmol) at 0° C. was added and the mixture was stirred for 15 min. The solvent was removed under reduced pressure and the resulting solid triturated with Et₂O anhydrous to afford the title compound E31a (0.032 g, 0.059 mmol, 89% yield) as a white solid. UPLC: rt=0.60 min, peak observed: 449 (M+1−HCl). C₂₅H₂₆ClFN₄OS requires 484.

### Example 31b

### 6-fluoro-8-methyl-2-({(2S)-1-[(2-methyl-5-phenyl-1,3-thiazol-4-yl)carbonyl]-2-piperidinyl}methyl)imidazo[1,2-a]pyridine (E31b)

In a 2 L reactor (vessel 1), 2-methyl-5-phenyl-1,3-thiazole-4-carboxylic acid (158 g, 0.72 mol) was suspended in isopropyl acetate (1 L) and potassium carbonate (190 g, 1.37 mol) was added. The mixture was stirred at 20° C. for 20 min. Pivaloyl chloride (92 ml, 0.75 mmol) was added and the mixture stirred for 30 min. In a 5 L reactor (vessel 2), 6-fluoro-8-methyl-2-[(2S)-2-piperidinylmethyl]imidazo[1,2-a]pyridine dihydrochloride D40b (200 g, 0.62 mol) was suspended in isopropyl acetate (1 L) followed by the addition of potassium carbonate (198 g, 1.42 mol) and water (1 L). The biphasic system was stirred at 20° C. for 20 min. The contents of vessel 1 were transferred into vessel 2, washing the line with isopropyl acetate (400 ml). The mixture was stirred at 20° C. for 2 h and then at 40° C. for 1 h. After cooling, the phases were allowed to separate (20 min). The aqueous phase was discharged. The organic phase was washed with water (2×1 L). The organic layer was concentrated under vacuo to 600 ml. The solution was aged at 20° C. for 14 h. Precipitation occurred. Heptane (2 L) was slowly added and the resulting light brown suspension was aged at 0° C. for 5 h. The solid was collected by filtration, washed with heptane/isopropyl acetate 85/15 (400 ml) and heptane (800 ml) and then dried at 40° C. for 18 h to afford the title compound E31 (249 g, 0.55 mol, 89% yield) as a pale brown solid. HPLC (walk-up, 3 min method): rt=1.95 min. ¹H NMR [the compound is present as a mixture of 2 conformers (ratio ca. 55/45), only one assigned] (600 MHz, DMSO-d₆) δ(ppm): 8.40-8.46 (m, 1H), 7.52 (s, 1H), 7.09-7.43 (m, 5H), 6.95 (d, 1H,), 4.40-4.50 (m, 1H), 3.97-4.10 (m, 1H), 2.94-3.17 (m, 2H), 2.70-2.78 (m, 1H), 2.51 (s, 3H), 2.30 (s, 3H), 0.82-1.78 (m, 6H).

### Example 32

### 8-ethenyl-6-fluoro-2-({(2S)-1-[(2-methyl-5-phenyl-1,3-thiazol-4-yl)carbonyl]-2-piperidinyl}methyl)imidazo[1,2-a]pyridine (HCl Salt) (E32)

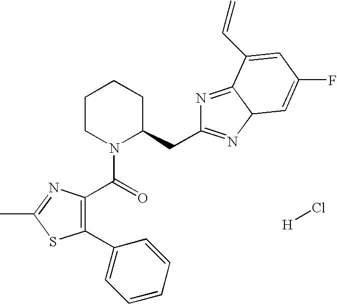

To a solution of 2-methyl-5-phenyl-1,3-thiazole-4-carboxylic acid (0.017 g, 0.08 mmol) in DCM (1 ml), oxalyl chloride (0.013 ml, 0.15 mmol) and a catalytic amount of anhydrous DMF were added. The solution was left under stirring for 1 h, then volatiles were removed under vacuum and the crude acyl chloride was dissolved in DCM (1 ml). The solution was added dropwise to an ice-cooled mixture of 8-ethenyl-6-fluoro-2-[(2S)-2-piperidinylmethyl]imidazo[1,2-a]pyridine D43 (0.016 g, 0.06 mmol) and TEA (0.044 ml, 0.31 mmol) in DCM (1 ml). The reaction mixture was left under stirring at room temperature for 1.5 h, then diluted with DCM and washed 3 times with a saturated NaHCO₃ aqueous solution. The organic layer was separated through a phase separator tube and concentrated. The crude (brown foam) was purified by chromatography on silica gel (Flash Master 10 g, DCM/MeOH from 100/0 to 99/1) and then by MDAP Fraction Lynx to afford the free base of the title compound (0.009 g, 0.02 mmol, 30% yield) as a white solid. UPLC: rt=0.63 min, peak observed: 461 (M+1). C₂₆H₂₅FN₄OS requires 460. The free base (0.009 g, 0.02 mmol) was dissolved in Et₂O (1 ml) and a 1 M HCl solution in Et₂O (0.30 ml, 0.30 mmol) was added at 0° C. The mixture was left under stirring at room temperature for 15 min. Volatiles were removed under vacuum and the residue triturated several times with Et₂O to give the title compound E32 (0.009 g, 0.02 mmol, 92% yield) as a white solid. HPLC (walk-up): rt=3.79 min.

UPLC: rt=0.63 min, peak observed: 461 (M+1−HCl). C₂₆H₂₆ClFN₄OS requires 496.

¹H NMR [the compound is present as a mixture of conformers (ratio ca. 70/30), only one assigned] (500 MHz, DMSO-d₆) δ(ppm): 8.91-9.17 (m, 1H), 7.93-8.40 (m, 1H), 7.13-7.46 (m, 6H), 7.07 (dd, 1H), 6.38 (d, 1H), 5.84 (d, 1H), 4.41-4.48 (m, 1H), 4.02-4.09 (m, 1H), 3.52-3.64 (m, 1H), 3.06-3.25 (m, 1H), 2.65-2.71 (m, 1H), 2.30 (s, 3H), 1.13-1.74 (m, 5H), 0.72-0.96 (m, 1H).

The following compounds of formula (V), where R represents a single substitution with R₂ or a substitution with R₂ and R₃, were prepared using a similar procedure to that described for Example 32. Each compound was obtained by amide coupling of the appropriate piperidine with 2-methyl-5-phenyl-1,3-thiazole-4-carboxylic acid.

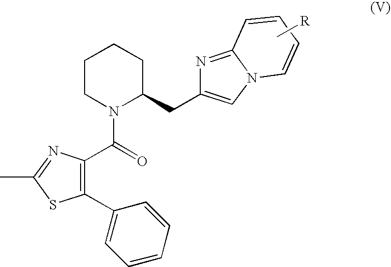

The compounds of examples 33 to 38 are as follows:

### Example 33 (E33)

8-ethyl-6-fluoro-2-({(2S)-1-[(2-methyl-5-phenyl-1,3-thiazol-4-yl)carbonyl]-2-piperidinyl} methyl)imidazo[1,2-a]pyridine (HCl salt);

### Example 34 (E34)

6-fluoro-8-(methyloxy)-2-({(2S)-1-[(2-methyl-5-phenyl-1,3-thiazol-4-yl)carbonyl]-2-piperidinyl}methyl)imidazo[1,2-a]pyridine (HCl salt);

### Example 35 (E35)

[6-fluoro-2-({(2S)-1-[(2-methyl-5-phenyl-1,3-thiazol-4-yl)carbonyl]-2-piperidinyl}methyl)imidazo[1,2-a]pyridin-8-yl]methanol (HCl salt);

### Example 36 (E36)

6-fluoro-8-[(methyloxy)methyl]-2-({(2S)-1-[(2-methyl-5-phenyl-1,3-thiazol-4-yl)carbonyl]-2-piperidinyl}methyl)imidazo[1,2-a]pyridine (HCl salt);

### Example 37 (E37)

8-chloro-2-({(2S)-1-[(2-methyl-5-phenyl-1,3-thiazol-4-yl)carbonyl]-2-piperidinyl}methyl)imidazo[1,2-a]pyridine (HCl salt);

### Example 38 (E38)

2-({(2S)-1-[(2-methyl-5-phenyl-1,3-thiazol-4-yl)carbonyl]-2-piperidinyl}methyl)-8-[(2,2,2-trifluoroethyl)oxy]imidazo[1,2-a]pyridine (HCl salt).

| | Piperidine | |
|---|---|---|
| No. | starting material | Characterising data |
| 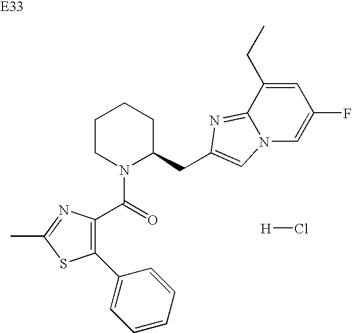 | D45 | Free base:HPLC (walk-up): rt = 4.19 min. UPLC: rt = 0.61min, peak observed: 463 (M + 1). C₂₆H₂₇FN₄OSrequires 462.HCl salt:HPLC (walk-up): rt = 4.19 min. UPLC: rt = 0.56min, peak observed: 463 (M + 1 − HCl).C₂₆H₂₈ClFN₄OS requires 498. |
| 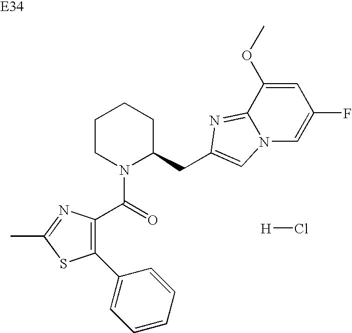 | D49 | Free base:HPLC (walk-up): rt = 3.67 min. UPLC: rt = 0.58min, peak observed: 465 (M + 1). C₂₅H₂₅FN₄O₂Srequires 464. ¹H NMR [the compound is present asa mixture of conformers (ratio ca. 50/50), only onassigned] (500 MHz, DMSO-d₆) δ(ppm): 8.20-8.24(m, 1 H), 7.46(s, 1 H), 7.20-7.41(m, 5 H),6.67(dd, 1 H), 4.42-4.49(m, 1 H), 3.85-3.92(m,4 H),2.93-3.13(m, 2 H), 2.75(dd, 1 H), 2.51(s, 3 H),1.25-1.78(m, 5 H), 0.83-1.06(m, 1 H).HCl salt:UPLC: rt = 0.58 min, peak observed: 465 (M + 1 −HCl). C₂₅H₂₆ClFN₄O₂S requires 500. |
| 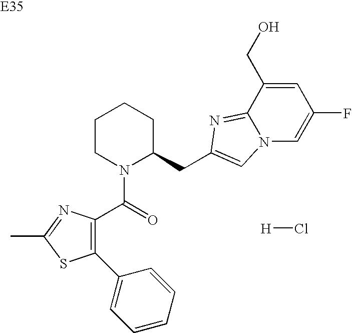 | D51 | Free base:HPLC (walk-up): rt = 3.50 min. UPLC: rt = 0.56min, peak observed: 465 (M + 1). C₂₅H₂₅FN₄O₂Srequires 464. ¹H NMR [the compound is present asa mixture of conformers (ratio ca. 55/45) and theassignment refers to the major component] (500MHz, DMSO-d₆) δ(ppm): 8.45-8.49(m, 1 H), 7.52s, 1 H), 7.03-7.42(m, 6 H), 5.43(t, 1 H),4.67(dd,1 H), 4.58(dd, 1 H), 4.44(d, 1 H), 3.94-4.02(m,1 H), 2.96-3.05(m, 2 H), 2.70(dd, 1 H), 2.48(s, 3 H),0.88-1.71(m, 6 H).HCl salt:HPLC (walk-up): rt = 3.52 min. |
| 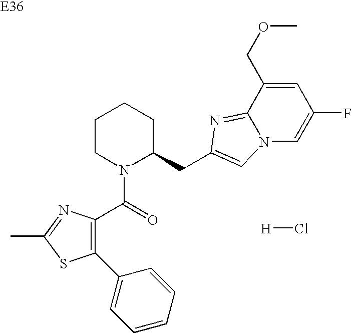 | D53 | Free base:HPLC (walk-up): rt = 3.73 min. UPLC: rt = 0.60min, peak observed: 479 (M + 1). C₂₆H₂₇FN₄O₂Srequires 478. ¹H NMR [the compound is present asa mixture of conformers (ratio ca. 55/45) and theassignment refers to the major component] (500MHz, DMSO-d₆) δ(ppm): 8.51-8.55(m, 1 H), 7.56(s, 1 H), 6.94-7.41(m, 6 H), 4.40-4.62(m,3 H),3.96-4.10(m, 1 H), 3.35(s, 3 H), 3.04-3.12(m, 1 H),2.95-3.04(m, 1 H0, 2.71(dd, 1 H), 2.51(s, 3 H),0.86-1.79(m, 6 H0.HCl salt:HPLC (walk-up): rt = 3.75 min. UPLC: rt = 0.61min, peak observed: 479 (M + 1 − HCl).C₂₆H₂₈ClFN₄O₂S requires 514. |
| 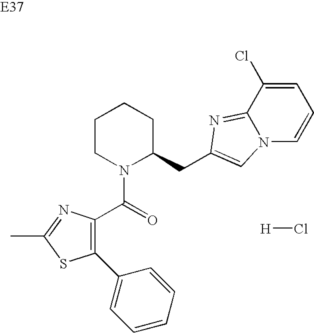 | D55 | Free base:UPLC: rt = 0.58 min, peaks observed: 451 (M + 1,100% and 453 (M + 1, 33%). C₂₄H₂₃ClN₄OS requires450. ¹H NMR [the compound is present as a mixtureof conformers (ratio ca. 55/45), only one assigned](500 MHz, DMSO-d₆)δ(ppm): 8.41(d, 1 H), 7.64(s, 1 H), 7.11-7.47(m,6 H), 6.75(t, 1 H), 4.50(dd, 1 H), 3.84-4.05(m,1 H), 2.83-3.28(m, 3 H),2.43(s, 3 H), 0.75-1.75(m, 6 H).HCl salt:HPLC (walk-up): rt = 3.56 min. MS: (ES/+) m/z:451 [M + 1 − HCl] and 453 [M + 1 − HCl].C₂₄H₂₄Cl₂N₄OS requires 486. |
| 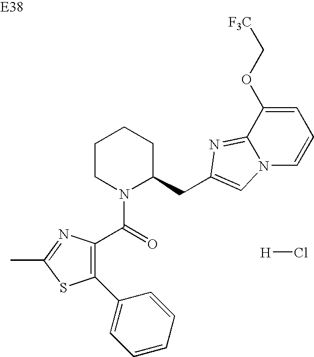 | D57 | Free base:UPLC: rt = 0.63 min, peaks observed: 515 (M + 1).C₂₆H₂₅F₃N₄O₂S requires 514. ¹H NMR [thecompound is present as a mixture of conformers(ratio ca. 55/45) and the assignment refers to themajor component] (500 MHz, DMSO-d₆) δ(ppm):8.12(dd, 1 H), 7.55(s, 1 H), 7.19-7.40(m, 5 H),6.65-6.72(m, 2 H), 4.84-4.95(m, 2 H), 4.45(dd,1 H),3.87-3.94(m, 1 H), 2.89-3.03(m, 2 H), 2.77(dd, 1 H), 2.44(s, 3 H), 1.21-1.73(m, 5 H), 0.85-0.98(m, 1 H).HCl salt:HPLC (walk-up): rt = 3.95 min. UPLC: rt = 0.63min, peak observed: 515 (M + 1 − HCl).C₂₆H₂₆ClF₃N₄O₂S requires 550. |

### Example 39

### 8-fluoro-2-[((2S)-1-{[5-(4-fluorophenyl)-2-methyl-1,3-thiazol-4-yl]carbonyl}-2-piperidinyl)methyl]imidazo[1,2-a]pyridine (HCl salt) (E39)

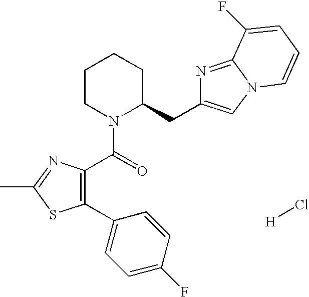

To a solution of 5-(4-fluorophenyl)-2-methyl-1,3-thiazole-4-carboxylic acid (0.39 g, 1.65 mmol) in DCM (5 ml), oxalyl chloride (0.32 ml, 3.63 mmol) and dry DMF (0.12 ml, 1.50 mml) were added. The mixture was left under stirring for 30 min and then concentrated under vacuum to provide a yellow/orange solid that was dissolved in DCM (5 ml). The acyl chloride solution was added dropwise to an ice-cooled mixture of 8-fluoro-2-[(2S)-2-piperidinylmethyl]imidazo[1,2-a]pyridine hydrochloride D58 (0.35 g, 1.50 mmol) and TEA (0.63 ml, 4.50 mmol) in DCM (5 ml). The reaction mixture was left under stirring at room temperature for 1 h, diluted with DCM (30 ml) and washed with a saturated NaHCO₃ aqueous solution (20 ml). The aqueous phase was back-extracted with DCM (2×5 ml). The organic layer was separated through a phase separator tube and the solvent removed under vacuum. The residue was purified on NH by flash chromatography (Biotage 40M, c-Hex/EtOAc from 100/0 to 20/80) to afford the free base of the title compound (0.52 g, 1.14 mmol, 76% yield) as a white solid. UPLC: rt=0.83 min, peak observed: 453 (M+1). C₂₄H₂₂F₂N₄OS requires 452.

¹H NMR [the compound is present as a mixture of conformers (ratio ca. 55/45) and the assignment refers to the major component] (500 MHz, DMSO-d₆) δ (ppm): 8.25 (dd, 1H), 7.66 (d, 1H), 7.21 (dd, 2H), 7.09 (t, 2H), 6.91-6.99 (m, 1H), 6.69-6.76 (m, 1H), 4.41-4.50 (m, 1H), 3.94-4.02 (m, 1H), 2.88-3.25 (m, 2H), 2.71-2.80 (m, 1H), 2.68 (s, 3H), 1.37-1.80 (m, 4H), 0.80-1.34 (m, 2H).

The free base (0.52 g, 1.14 mmol) was dissolved in DCM (3 ml) and a 1M HCl solution in Et₂O (1.50 ml, 1.50 mmol) was added at 0° C. The mixture was left under stirring at room temperature for 30 min. Volatiles were removed under vacuum and the residue triturated with Et₂O (3 ml). After the solvent removal, the residue was dried at 50° C. under reduced pressure for 48 h to afford the title compound E39 (0.56 g, 1.14 mmol, 76% yield from D19, two steps) as a white solid.

MS: (ES/+) m/z: 453 (M+1−HCl). C₂₄H₂₃ClF₂N₄OS requires 486.

The following compounds of formula (VI), where X represents H or F and R represents a single substitution with R₂ or a substitution with R₂ and R₃, were prepared using a similar procedure to that described for Example 39. Each compound was obtained by amide coupling between the appropriate piperidine and 2-methyl-5-aryl-1,3-thiazole-4-carbonyl chloride. This is provided merely for assistance to the skilled chemist.

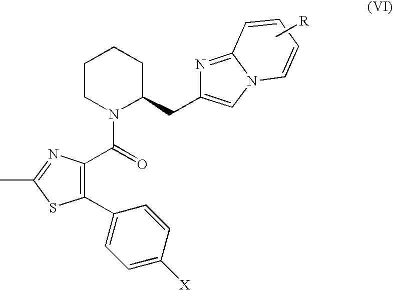

The compounds of examples 40 to 42 are as follows:

### Example 40 (E40)

8-fluoro-3-methyl-2-({(2S)-1-[(2-methyl-5-phenyl-1,3-thiazol-4-yl)carbonyl]-2-piperidinyl}methyl)imidazo[1,2-a]pyridine (HCl salt);

### Example 41 (E41)

8-fluoro-2-[((2S)-1-{[5-(4-fluorophenyl)-2-methyl-1,3-thiazol-4-yl]carbonyl}-2-piperidinyl)methyl]-3-methylimidazo[1,2-a]pyridine (HCl salt);

### Example 42 (E42)

3-chloro-8-methyl-2-({(2S)-1-[(2-methyl-5-phenyl-1,3-thiazol-4-yl)carbonyl]-2-piperidinyl}methyl)imidazo[1,2-a]pyridine hydrochloride (HCl salt).

| | Amide coupling | |
|---|---|---|
| No. | Reactants | Characterising data |
| 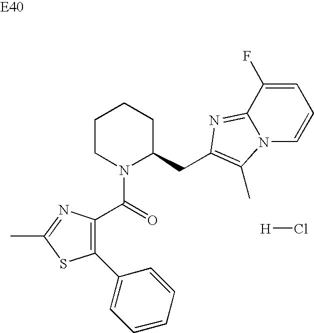 | D61 and 2-methyl-5-phenyl-1,3-thiazole-4-carbonyl chloride | Free base:UPLC: rt = 0.85 min, peak observed: 449 (M + 1).C₂₅H₂₅FN₄OS requires 448. ¹H NMR [the compoundis present as a mixture of conformers (ratio ca. 60/40),only one assigned] (500 MHz, DMSO-d₆) δ(ppm):7.95-8.18(m, 1 H), 7.20-7.45(m, 5 H), 6.96-7.19(m,1 H), 6.75-6.95(m, 1 H), 4.46(bd, 1 H), 3.80-3.91(m,1 H), 2.99-3.26(m, 2 H), 2.74(dd, 1 H), 2.37(s, 3 H),2.24(s, 3 H), 0.65-1.80(m, 6 H).HCl salt:HPLC (walk-up): rt = 4.74 min. MS: (ES/+) m/z: 449(M + 1 − HCl). C₂₅H₂₆ClFN₄OS requires 484. |
| 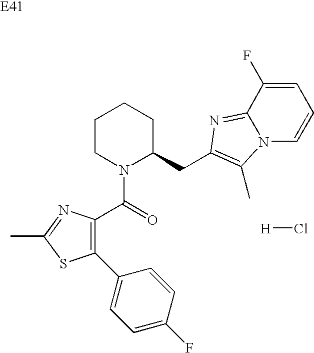 | D61 and 5-(4-fluorophenyl)-2-methyl-1,3-thiazole-4-carbonyl chloride | Free base:UPLC: rt = 0.86 min, peak observed: 467 (M + 1).C₂₅H₂₄F₂N₄OS requires 466. ¹H NMR [the compoundis present as a mixture of conformers (ratio ca. 60/40),only one assigned] (500 MHz, DMSO-d₆) δ(ppm):7.90-8.03(m, 1 H), 7.08-7.48(m, 4 H), 6.73-7.07(m,2 H), 4.46(bd, 1 H), 3.80-3.91(m, 1 H), 2.99-3.26(m,2 H), 2.74(dd, 1 H), 2.41(s, 3 H), 2.24(s, 3 H), 0.65-1.80(m, 6 H).HCl salt:HPLC (walk-up): rt = 4.78 min. MS: (ES/+) m/z: 467(M + 1 − HCl). C₂₅H₂₅ClF₂N₄OS requires 502. |
| 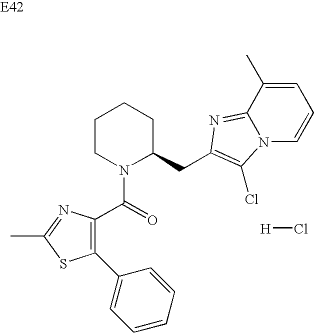 | D63 and 2-methyl-5-phenyl-1,3-thiazole-4-carbonyl chloride | Free base:HPLC (walk-up): rt = 3.85 min. MS: (ES/+) m/z: 465(M + 1). C₂₅H₂₅ClN₄OS requires 464. ¹H NMR [thecompound is present as a mixture of conformers (ratioca. 70/30) and the assignment refers to the majorcomponent] (500 MHz, DMSO-d₆) δ(ppm): 8.02(d, 1 H),7.14-7.47(m, 5 H), 7.04(d, 1 H), 6.89(t, 1 H),4.48(dd, 1 H), 3.91-4.09(m, 1 H), 2.95-3.27(m, 2 H),2.71(dd, 1 H), 2.31-2.40(m, 6 H), 0.71-1.77(m,6 H).HCl salt:HPLC (walk-up): rt = 4.78 min. MS: (ES/+) m/z: 467(M + 1 − HCl). C₂₅H₂₅ClF₂N₄OS requires 502. |

### Example 43

### Determination of Antagonist Affinity at Human Orexin-1 and 2 Receptors Using FLIPR

### Cell Culture

Adherent Chinese Hamster Ovary (CHO) cells, stably expressing the recombinant human Orexin-1 or human Orexin-2 receptors or Rat Basophilic Leukaemia Cells (RBL) stably expressing recombinant rat Orexin-1 or rat Orexin-2 receptors were maintained in culture in Alpha Minimum Essential Medium (Gibco/Invitrogen, cat. no.; 22571-020), supplemented with 10% decomplemented foetal bovine serum (Life Technologies, cat. no. 10106-078) and 400 μg/mL Geneticin G418 (Calbiochem, cat. no. 345810). Cells were grown as monolayers under 95%:5% air:CO₂ at 37° C.

The sequences of the human orexin 1, human orexin 2, rat orexin 1 and rat orexin 2 receptors used in this example were as published in Sakurai, T. et al (1998) Cell, 92 pp 573 to 585, with the exception that the human orexin 1 receptor sequence used had the amino acid residue alanine at position 280 and not glycine as reported in Sakurai et al.

Measurement of [Ca²⁺]ᵢ using the FLIPR™

Cells were seeded into black clear-bottom 384-well plates (density of 20,000 cells per well) in culture medium as described above and maintained overnight (95%:5% air:CO₂ at 37° C.). On the day of the experiment, culture medium were discarded and the cells washed three times with standard buffer (NaCl, 145 mM; KCl, 5 mM; HEPES, 20 mM; Glucose, 5.5 mM; MgCl₂, 1 mM; CaCl₂, 2 mM) added with Probenecid 2.5 mM. The plates were then incubated at 37° C. for 60 minutes in the dark with 1 μM FLUO-4AM dye to allow cell uptake of the FLUO-4AM, which is subsequently converted by intracellular esterases to FLUO-4, which is unable to leave the cells. After incubation, cells were washed three times with standard buffer to remove extracellular dye and 30 μL of buffer were left in each well after washing.

Compounds of the invention were tested in a final assay concentration range from 1.66×10⁻⁵M to 1.58×10⁻¹¹ M. Compounds of the invention were dissolved in dimethylsulfoxide (DMSO) at a stock concentration of 10 mM. These stock solutions were serially diluted with DMSO and 1 μL of each dilution was transferred to a 384 well compound plate. Immediately before introducing compound to the cells, buffer solution (50 μl/well) was added to this plate. To allow agonist stimulation of the cells, a stock plate containing a solution of human orexin A (hOrexin A) was diluted with buffer to final concentration just before use. This final concentration of hOrexin A was equivalent to the calculated EC80 for hOrexinA agonist potency in this test system. This value was obtained by testing hOrexinA in concentration response curve (at least 16 replicates) the same day of the experiment.

The loaded cells were then incubated for 10 min at 37° C. with test compound. The plates were then placed into a FLIPR™ (Molecular Devices, UK) to monitor cell fluorescence (λₑₓ=488 nm, λ_{EM}=540 nm) (Sullivan E, Tucker E M, Dale I L. Measurement of [Ca²⁺]ᵢ using the fluometric imaging plate reader (FLIPR). In: Lambert DG (ed.), Calcium Signaling Protocols. New Jersey: Humana Press, 1999, 125-136). A baseline fluorescence reading was taken over a 5 to 10 second period, and then 10 μL of EC80 hOrexinA solution was added. The fluorescence was then read over a 45 minute period.

### Data Analysis

Functional responses using FLIPR were measured as peak fluorescence intensity minus basal fluorescence and expressed as a percentage of a non-inhibited Orexin-A-induced response on the same plate. Iterative curve-fitting and parameter estimations were carried out using a four parameter logistic model and Microsoft Excel (Bowen W P, Jerman J C. Nonlinear regression using spreadsheets. Trends Pharmacol. Sci. 1995; 16: 413-417). Antagonist affinity values (IC₅₀) were converted to functional pKᵢ values using a modified Cheng-Prusoff correction (Cheng Y C, Prusoff W H. Relationship between the inhibition constant (Kᵢ) and the concentration of inhibitor which causes 50 percent inhibition (IC₅₀) of an enzymatic reaction. Biochem. Pharmacol. 1973, 22: 3099-3108).

$$fpKi = - log \frac{( {IC}_{50} )}{{( 2 + {( \frac{[ agonist ]}{( {EC}_{50} )} )}^{n} )}^{1 / n} - 1}$$

Where [agonist] is the agonist concentration, EC₅₀ is the concentration of agonist giving 50% activity derived from the agonist dose response curve and n=slope of the dose response curve. When n=1 the equation collapses to the more familiar Cheng-Prusoff equation.

Compounds of examples 1 to 42 were tested according to the method of example 43. All compounds gave fpKi values from 8.0 to 10.0 at the human cloned orexin-1 receptor (having the amino acid residue alanine at position 280 and not glycine) and from 6.1 to 9.4 at the human cloned orexin-2 receptor.

Compounds of examples 13 and 31 were tested according to the method of example 43 on cloned rat OX1 receptor and cloned rat OX2 receptors and gave fpKi values from 9.0 to 8.3 and 9.0 to 9.5 respectively.

What is claimed is:

1. A compound which is 6-fluoro-8-methyl-2-({(2S)-1-[(2-methyl-5-phenyl-1,3-thiazol-4-yl)carbonyl]-2-piperidinyl}methyl)imidazo[1,2-a]pyridine or a pharmaceutically acceptable salt thereof.

2. A compound which is 6-fluoro-8-methyl-2-({(2S)-1-[(2-methyl-5-phenyl-1,3-thiazol-4-yl)carbonyl]-2-piperidinyl}methyl)imidazo[1,2-a]pyridine.

3. A compound which is a hydrochloride salt of 6-fluoro-8-methyl-2-({(2S)-1-[(2-methyl-5-phenyl-1,3-thiazol-4-yl)carbonyl]-2-piperidinyl}methyl)imidazo[1,2-a]pyridine.

4. A method of treating or preventing a disease or disorder where an antagonist of a human orexin receptor is required, which comprises administering to a human in need thereof an effective amount of the compound, or pharmaceutically acceptable salt thereof, according to claim 1, wherein the disease or disorder is a sleep disorder.

5. A method of treating or preventing a disease or disorder where an antagonist of a human orexin receptor is required, which comprises administering to a human in need thereof an effective amount of the compound according to claim 2, wherein the disease or disorder is a sleep disorder.

6. A method of treating or preventing a disease or disorder where an antagonist of a human orexin receptor is required, which comprises administering to a human in need thereof an effective amount of the compound according to claim 3, wherein the disease or disorder is a sleep disorder.

7. A method according to claim 4, where the sleep disorder is selected from the group consisting of Primary Insomnia, Breathing-Related Sleep Disorders, Circadian Rhythm Sleep Disorder, Nightmare Disorder, Sleep Terror Disorder, Sleepwalking Disorder, Insomnia Related to Another Mental Disorder, and Jet-Lag Syndrome.

8. A method according to claim 5, where the sleep disorder is selected from the group consisting of Primary Insomnia, Breathing-Related Sleep Disorders, Circadian Rhythm Sleep Disorder, Nightmare Disorder, Sleep Terror Disorder, Sleepwalking Disorder, Insomnia Related to Another Mental Disorder, and Jet-Lag Syndrome.

9. A method according to claim 6, where the sleep disorder is selected from the group consisting of Primary Insomnia, Breathing-Related Sleep Disorders, Circadian Rhythm Sleep Disorder, Nightmare Disorder, Sleep Terror Disorder, Sleepwalking Disorder, Insomnia Related to Another Mental Disorder, and Jet-Lag Syndrome.

10. A method of treating insomnia which comprises administering to a human in need thereof an effective amount of the compound, or pharmaceutically acceptable salt thereof, according to claim 1.

11. A method of treating insomnia which comprises administering to a human in need thereof an effective amount of the compound according to claim 2.

12. A method of treating insomnia which comprises administering to a human in need thereof an effective amount of the compound according to claim 3.

13. A method preventing a sleep disorder selected from jet-lag-syndrome and circadian rhythm sleep disorder, which method comprises administering to a human in need thereof an effective amount of the compound, or pharmaceutically acceptable salt thereof, according to claim 1.

14. A method of preventing a sleep disorder selected from jet-lag-syndrome and circadian rhythm sleep disorder, which method comprises administering to a human in need thereof an effective amount of the compound according to claim 2.

15. A method of preventing a sleep disorder selected from jet-lag-syndrome and circadian rhythm sleep disorder, which method comprises administering to a human in need thereof an effective amount of the compound according to claim 3.

16. A pharmaceutical composition comprising a compound which is 6-fluoro-8-methyl-2-({(2S)-1-[(2-methyl-5-phenyl-1,3-thiazol-4-yl)carbonyl]-2-piperidinyl}methyl)imidazo[1,2-a]pyridine or a pharmaceutically acceptable salt thereof, and a pharmaceutically acceptable carrier.

17. A pharmaceutical composition comprising a compound which is 6-fluoro-8-methyl-2-({(2S)-1-[(2-methyl-5-phenyl-1,3-thiazol-4-yl)carbonyl]-2-piperidinyl}methyl)imidazo[1,2-a]pyridine and a pharmaceutically acceptable carrier.

18. A pharmaceutical composition comprising a compound which is a hydrochloride salt of 6-fluoro-8-methyl-2-({(2S)-1-[(2-methyl-5-phenyl-1,3-thiazol-4-yl)carbonyl]-2-piperidinyl}methyl)imidazo[1,2-a]pyridine, and a pharmaceutically acceptable carrier.

19. A pharmaceutical composition according to claim 16, wherein said composition is adapted for oral administration or nasal administration.

20. A pharmaceutical composition according to claim 17, wherein said composition is adapted for oral administration or nasal administration.

21. A pharmaceutical composition according to claim 18, wherein said composition is adapted for oral administration or nasal administration.

22. A pharmaceutical composition according to claim 19, wherein said composition adapted for oral administration is in the form of a tablet or a capsule.

23. A pharmaceutical composition according to claim 20, wherein said composition adapted for oral administration is in the form of a tablet or a capsule.

24. A pharmaceutical composition according to claim 21, wherein said composition adapted for oral administration is in the form of a tablet or a capsule.

25. A pharmaceutical composition according to claim 19, wherein said composition adapted for nasal administration is in the form of an aerosol, drops, a gel or a powder.

26. A pharmaceutical composition according to claim 20, wherein said composition adapted for nasal administration is in the form of an aerosol, drops, a gel or a powder.

27. A pharmaceutical composition according to claim 21, wherein said composition adapted for nasal administration is in the form of an aerosol, drops, a gel or a powder.

28. A method of treating or preventing a disease or disorder where an antagonist of a human orexin receptor is required, which comprises administering to a human in need thereof an effective amount of the pharmaceutical composition according to claim 16, wherein the disease or disorder is a sleep disorder.

29. A method of treating or preventing a disease or disorder where an antagonist of a human orexin receptor is required, which comprises administering to a human in need thereof an effective amount of the pharmaceutical composition according to claim 17, wherein the disease or disorder is a sleep disorder.

30. A method of treating or preventing a disease or disorder where an antagonist of a human orexin receptor is required, which comprises administering to a human in need thereof an effective amount of the pharmaceutical composition according to claim 18, wherein the disease or disorder is a sleep disorder.

31. A method according to claim 28, where the sleep disorder is selected from the group consisting of Primary Insomnia, Breathing-Related Sleep Disorders, Circadian Rhythm Sleep Disorder, Nightmare Disorder, Sleep Terror Disorder, Sleepwalking Disorder, Insomnia Related to Another Mental Disorder, and Jet-Lag Syndrome.

32. A method according to claim 29, where the sleep disorder is selected from the group consisting of Primary Insomnia, Breathing-Related Sleep Disorders, Circadian Rhythm Sleep Disorder, Nightmare Disorder, Sleep Terror Disorder, Sleepwalking Disorder, Insomnia Related to Another Mental Disorder, and Jet-Lag Syndrome.

33. A method according to claim 30, where the sleep disorder is selected from the group consisting of Primary Insomnia, Breathing-Related Sleep Disorders, Circadian Rhythm Sleep Disorder, Nightmare Disorder, Sleep Terror Disorder, Sleepwalking Disorder, Insomnia Related to Another Mental Disorder, and Jet-Lag Syndrome.

34. A method of treating insomnia which comprises administering to a human in need thereof an effective amount of the pharmaceutical composition according to claim 16.

35. A method of treating insomnia which comprises administering to a human in need thereof an effective amount of the pharmaceutical composition according to claim 17.

36. A method of treating insomnia which comprises administering to a human in need thereof an effective amount of the pharmaceutical composition according to claim 18.

37. A method preventing a sleep disorder selected from jet-lag-syndrome and circadian rhythm sleep disorder, which method comprises administering to a human in need thereof an effective amount of the pharmaceutical composition according to claim 16.

38. A method preventing a sleep disorder selected from jet-lag-syndrome and circadian rhythm sleep disorder, which method comprises administering to a human in need thereof an effective amount of the pharmaceutical composition according to claim 17.

39. A method preventing a sleep disorder selected from jet-lag-syndrome and circadian rhythm sleep disorder, which method comprises administering to a human in need thereof an effective amount of the pharmaceutical composition according to claim 18.
